(19) United States
(12) Patent Application Publication
(10) Pub. No.: US 2003/0018182 A1
(43) Pub. Date: Jan. 23, 2003

(54) Non-endogenous, constitutively activated human G protein-coupled receptors

(76) Inventors: Dominic P. Behan, San Diego, CA (US); Karin Lehmann-Bruinsma, San Diego, CA (US); Derek T. Chalmers, Cardiff, CA (US); Ruoping Chen, San Deigo, CA (US); Huong T. Dang, San Deigo, CA (US); Martin Gore, San Diego, CA (US); Chen W. Liaw, San Diego, CA (US); I-Lin Lin, San Diego, CA (US); Kevin Lowitz, San Diego, CA (US); Carol White, San Diego, CA (US)

Correspondence Address: Woodcock Washburn Kurtz, Mackiewicz & Norris LLP, One Liberty Place - 46th Floor, Philadelphia, PA, 19103, US

(21) Appl. No.: 09/876,252

(22) Filed: Jun. 7, 2001

Related U.S. Application Data
(62) Division of application No. 09/416,760, filed on Oct. 12, 1999.
Application No. 09/416,760 is a continuation-in-part of application No. 09/170,496, filed on Oct. 13, 1998.
(60) Provisional application No. 60/110,060, filed on Nov. 27, 1998.
(60) Provisional application No. 60/120,416, filed on Feb. 16, 1999.
(60) Provisional application No. 60/121,852, filed on Feb. 26, 1999.
(60) Provisional application No. 60/109,213, filed on Nov. 20, 1998.
(60) Provisional application No. 60/123,944, filed on Mar. 12, 1999.
(60) Provisional application No. 60/123,945, filed on Mar. 12, 1999.
(60) Provisional application No. 60/123,948, filed on Mar. 12, 1999.
(60) Provisional application No. 60/123,951, filed on Mar. 12, 1999.
(60) Provisional application No. 60/123,946, filed on Mar. 12, 1999.
(60) Provisional application No. 60/123,949, filed on Mar. 12, 1999.
(60) Provisional application No. 60/152,524, filed on Sep. 3, 1999.
(60) Provisional application No. 60/151,114, filed on Aug. 27, 1999.
(60) Provisional application No. 60/108,029, filed on Nov. 12, 1998.
(60) Provisional application No. 60/136,436, filed on May 28, 1999.
(60) Provisional application No. 60/136,439, filed on May 28, 1999.
(60) Provisional application No. 60/136,567, filed on May 28, 1999.
(60) Provisional application No. 60/137,127, filed on May 28, 1999.
(60) Provisional application No. 60/137,131, filed on May 28, 1999.
(60) Provisional application No. 60/141,448, filed on Jun. 29, 1999.
(60) Provisional application No. 60/136,437, filed on May 28, 1999.
(60) Provisional application No. 60/156,555, filed on Sep. 29, 1999.
(60) Provisional application No. 60/156,634, filed on Sep. 29, 1999.
(60) Provisional application No. 60/156,653, filed on Sep. 29, 1999.
(60) Provisional application No. 60/157,280, filed on Oct. 1, 1999.
(60) Provisional application No. 60/157,294, filed on Oct. 1, 1999.
(60) Provisional application No. 60/157,281, filed on Oct. 1, 1999.
(60) Provisional application No. 60/157,293, filed on Oct. 1, 1999.
(60) Provisional application No. 60/157,282, filed on Oct. 1, 1999.

(51) Int. Cl. C07H 21/04; C12N 5/06; C07K 14/705; C12P 21/02

(52) U.S. Cl. 536/23.5; 530/350; 435/69.1; 435/320.1; 435/325

(57) ABSTRACT

The invention disclosed in this patent document relates to transmembrane receptors, more particularly to a human G protein-coupled receptor for which the endogenous ligand is unknown (“orphan GPCR receptors”), and most particularly to mutated (non-endogenous) versions of the human GPCRs for evidence of constitutive activity.

This patent application is a continuation-in-part of, and claims priority from, U.S. Ser. No. 09/170,496, filed with the U.S. patent and Trademark Office on Oct. 13, 1998. This application also claims the benefit of priority from the following provisional applications, all filed via U.S. Express Mail with the U.S. patent and Trademark Office on the indicated dates: U.S. Provisional No. 60/110,060, filed Nov. 27, 1998; U.S. Provisional No. 60/120,416, filed Feb. 16, 1999; U.S. Provisional No. 60/121,852, filed Feb. 26, 1999 claiming benefit of U.S. Provisional No. 60/109,213, filed Nov. 20, 1998; U.S. Provisional No. 60/123,944, filed Mar. 12, 1999; U.S. Provisional No. 60/123,945, filed Mar. 12, 1999; U.S. Provisional No. 60/123,948, filed Mar. 12, 1999; U.S. Provisional No. 60/123,951, filed Mar. 12, 1999; U.S. Provisional No. 60/123,946, filed Mar. 12, 1999; U.S. Provisional No. 60/123,949, filed Mar. 12, 1999; U.S. Provisional No. 60/152,524, filed Sep. 3, 1999, claiming benefit of U.S. Provisional No. 60/151,114, filed Aug. 27, 1999 and U.S. Provisional No. 60/108,029, filed Nov. 12, 1998; U.S. Provisional No. 60/136,436, filed May 28, 1999; U.S. Provisional No. 60/136,439, filed May 28, 1999; U.S. Provisional No. 60/136,567, filed May 28, 1999; U.S. Provisional No. 60/137,127, filed May 28, 1999; U.S. Provisional No. 60/137,131, filed May 28, 1999; U.S. Provisional No. 60/141,448, filed Jun. 29, 1999 claiming benefit of U.S. Provisional No. 60/136,437, filed May 28, 1999; U.S. Provisional No. 60/156,633, filed Sep. 29, 1999; U.S. Provisional No. 60/156,555, filed Sep. 29, 1999; U.S. Provisional No. 60/156,634, filed Sep. 29, 1999;U.S. Provisional No. ______ (Arena Pharmaceuticals, Inc. docket number: CHN10-1), filed Sep. 29, 1999; U.S. Provisional No. ______ (Arena Pharmaceuticals, Inc. docket number: RUP6-1), filed Oct. 1, 1999; U.S. Provisional No. ______ (Arena Pharmaceuticals, Inc. docket number: RUP7-1), filed Oct. 1, 1999; U.S. Provisional No. ______ (Arena Pharmaceuticals, Inc. docket number: CHN6-1), filed Oct. 1, 1999; U.S. Provisional No. ______ (Arena Pharmaceuticals, Inc. docket number: RUP5-1), filed Oct. 1, 1999; and U.S. Provisional No. ______ (Arena Pharmaceuticals, Inc. docket number: CHN9-1), filed Oct. 1, 1999. This application is also related to co-pending U.S. Ser. No. ______ (Woodcock, Washburn, Kurtz, Makiewicz & Norris, LLP docket number AREN-0050), filed on Oct. 12, 1999 (via U.S. Express Mail) and U.S. Ser. No. 09/364,425, filed on Jul. 30, 1999, both incorporated herein by reference. Each of the foregoing applications are incorporated by reference herein in their entirety.

### FIELD OF THE INVENTION

The invention disclosed in this patent document relates to transmembrane receptors, and more particularly to human G protein-coupled receptors, and specifically to GPCRs that have been altered to establish or enhance constitutive activity of the receptor. Preferably, the altered GPCRs are used for the direct identification of candidate compounds as receptor agonists, inverse agonists or partial agonists having potential applicability as therapeutic agents.

### BACKGROUND OF THE INVENTION

Although a number of receptor classes exist in humans, by far the most abundant and therapeutically relevant is represented by the G protein-coupled receptor (GPCR or GPCRs) class. It is estimated that there are some 100,000 genes within the human genome, and of these, approximately 2%, or 2,000 genes, are estimated to code for GPCRs. Receptors, including GPCRs, for which the endogenous ligand has been identified are referred to as “known” receptors, while receptors for which the endogenous ligand has not been identified are referred to as “orphan” receptors. GPCRs represent an important area for the development of pharmaceutical products: from approximately 20 of the 100 known GPCRs, 60% of all prescription pharmaceuticals have been developed.

GPCRs share a common structural motif. All these receptors have seven sequences of between 22 to 24 hydrophobic amino acids that form seven alpha helices, each of which spans the membrane (each span is identified by number, i.e., transmembrane-1 (TM-1), transmebrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when an endogenous ligand binds with the receptor (often referred to as “activation” of the receptor), there is a change in the conformation of the intracellular region that allows for coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz and Go are G proteins that have been identified. Endogenous ligand-activated GPCR coupling with the G-protein begins a signaling cascade process (referred to as “signal transduction”). Under normal conditions, signal transduction ultimately results in cellular activation or cellular inhibition. It is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to produce a biological response. Changing the receptor conformation to the active state allows linkage to the transduction pathway (via the G-protein) and produces abiological response.

A receptor may be stabilized in an active state by an endogenous ligand or a compound such as a drug. Recent discoveries, including but not exclusively limited to modifications to the amino acid sequence of the receptor, provide means other than endogenous ligands or drugs to promote and stabilize the receptor in the active state conformation. These means effectively stabilize the receptor in an active state by simulating the effect of an endogenous ligand binding to the receptor. Stabilization by such ligand-independent means is termed “constitutive receptor activation.”

### SUMMARY OF THE INVENTION

Disclosed herein are non-endogenous versions of endogenous, human GPCRs and uses thereof.

### BRIEF DESCRIPTION OF THE DRAWINGS

FIG. 1 is a representation of 8XCRE-Luc reporter plasmid (see, Example 4(c)3.)

FIGS. 2A and 2B are graphic representations of the results of ATP and ADP binding to endogenous TDAG8 (2A) and comparisons in serum and serum free media (2B).

FIG. 3 is a graphic representation of the comparative signaling results of CMV versus the GPCR Fusion Protein H9(F236K):Gsa.

### DETAILED DESCRIPTION

The scientific literature that has evolved around receptors has adopted a number of terms to refer to ligands having various effects on receptors. For clarity and consistency, the following definitions will be used throughout this patent document. To the extent that these definitions conflict with other definitions for these terms, the following definitions shall control:

AGONISTS shall mean materials (e.g., ligands, candidate compounds) that activate the intracellular response when they bind to the receptor, or enhance GTP binding to membranes.

TABLE A
AMINO ACID ABBREVIATIONS used herein are set out in Table A:

| | | |
|---|---|---|
| ALANINE | ALA | A |
| ARGININE | ARG | R |
| ASPARAGNE | ASN | N |
| ASPARTIC ACID | ASP | D |
| CYSTEINE | CYS | C |
| GLUTAMIC ACID | GLU | E |
| GLUTAMINE | GLN | Q |
| GLYCINE | GLY | G |
| HISTIDINE | HIS | H |
| ISOLEUCINE | ILE | I |
| LEUCINE | LEU | L |
| LYSINE | LYS | K |
| METHIONINE | MET | M |
| PHENYLALANINE | PHE | F |
| PROLINE | PRO | P |
| SERINE | SER | S |
| THREONINE | THR | T |
| TRYPTOPHAN | TRP | W |
| TYROSINE | TYR | Y |
| VALINE | VAL | V |

PARTIAL AGONISTS shall mean materials (e.g., ligands, candidate compounds) that activate the intracellular response when they bind to the receptor to a lesser degree/extent than do agonists, or enhance GTP binding to membranes to a lesser degree/extent than do agonists.

ANTAGONIST shall mean materials (e.g., ligands, candidate compounds) that competitively bind to the receptor at the same site as the agonists but which do not activate the intracellular response initiated by the active form of the receptor, and can thereby inhibit the intracellular responses by agonists or partial agonists. ANTAGONISTS do not diminish the baseline intracellular response in the absence of an agonist or partial agonist.

CANDIDATE COMPOUND shall mean a molecule (for example, and not limitation, a chemical compound) that is amenable to a screening technique. Preferably, the phrase “candidate compound” does not include compounds which were publicly known to be compounds selected from the group consisting of inverse agonist, agonist or antagonist to a receptor, as previously determined by an indirect identification process (“indirectly identified compound”); more preferably, not including an indirectly identified compound which has previously been determined to have therapeutic efficacy in at least one mammal; and, most preferably, not including an indirectly identified compound which has previously been determined to have therapeutic utility in humans.

COMPOSITION means a material comprising at least one component; a “pharmaceutical composition” is an example of a composition.

COMPOUND EFFICACY shall mean a measurement of the ability of a compound to inhibit or stimulate receptor functionality, as opposed to receptor binding affinity. Exemplary means of detecting compound efficacy are disclosed in the Example section of this patent document.

CODON shall mean a grouping of three nucleotides (or equivalents to nucleotides) which generally comprise a nucleoside (adenosine (A), guanosine (G), cytidine (C), uridine (U) and thymidine (T)) coupled to a phosphate group and which, when translated, encodes an amino acid.

CONSTITUTIVELY ACTIVATED RECEPTOR shall mean a receptor subject to constitutive receptor activation. A constitutively activated receptor can be endogenous or non-endogenous.

CONSTITUTIVE RECEPTOR ACTIVATION shall mean stabilization of a receptor in the active state by means other than binding of the receptor with its endogenous ligand or a chemical equivalent thereof.

CONTACT or CONTACTING shall mean bringing at least two moieties together, whether in an in vitro system or an in vivo system.

DIRECTLY IDENTIFYING or DIRECTLY IDENTIFIED, in relationship to the phrase “candidate compound”, shall mean the screening of a candidate compound against a constitutively activated receptor, preferably a constitutively activated orphan receptor, and most preferably against a constitutively activated G protein-coupled cell surface orphan receptor, and assessing the compound efficacy of such compound. This phrase is, under no circumstances, to be interpreted or understood to be encompassed by or to encompass the phrase “indirectly identifying” or “indirectly identified.”

ENDOGENOUS shall mean a material that a mammal naturally produces. ENDOGENOUS in reference to, for example and not limitation, the term “receptor,” shall mean that which is naturally produced by a mammal (for example, and not limitation, a human) or a virus. By contrast, the term NON-ENDOGENOUS in this context shall mean that which is not naturally produced by a mammal (for example, and not limitation, a human) or a virus. For example, and not limitation, a receptor which is not constitutively active in its endogenous form, but when manipulated becomes constitutively active, is most preferably referred to herein as a “non-endogenous, constitutively activated receptor.” Both terms can be utilized to describe both “in vivo” and “in vitro” systems. For example, and not limitation, in a screening approach, the endogenous or non-endogenous receptor may be in reference to an in vitro screening system. As a further example and not limitation, where the genome of a mammal has been manipulated to include a non-endogenous constitutively activated receptor, screening of a candidate compound by means of an in vivo system is viable.

G PROTEIN COUPLED RECEPTOR FUSION PROTEIN and GPCR FUSION PROTEIN, in the context of the invention disclosed herein, each mean a non-endogenous protein comprising an endogenous, constitutively activate GPCR or a non-endogenous, constitutively activated GPCR fused to at least one G protein, most preferably the alpha (a) subunit of such G protein (this being the subunit that binds GTP), with the G protein preferably being of the same type as the G protein that naturally couples with endogenous orphan GPCR. For example, and not limitation, in an endogenous state, if the G protein “Gsa” is the predominate G protein that couples with the GPCR, a GPCR Fusion Protein based upon the specific GPCR would be a non-endogenous protein comprising the GPCR fused to Gsa; in some circumstances, as will be set forth below, a non-predominant G protein can be fused to the GPCR. The G protein can be fused directly to the c-terminus of the constitutively active GPCR or there may be spacers between the two.

HOST CELL shall mean a cell capable of having a Plasmid and/or Vector incorporated therein. In the case of a prokaryotic Host Cell, a Plasmid is typically replicated as a autonomous molecule as the Host Cell replicates (generally, the Plasmid is thereafter isolated for introduction into a eukaryotic Host Cell); in the case of a eukaryotic Host Cell, a Plasmid is integrated into the cellular DNA of the Host Cell such that when the eukaryotic Host Cell replicates, the Plasmid replicates. Preferably, for the purposes of the invention disclosed herein, the Host Cell is eukaryotic, more preferably, mammalian, and most preferably selected from the group consisting of 293, 293T and COS-7 cells.

INDIRECTLY IDENTIFYING or INDIRECTLY IDENTIFIED means the traditional approach to the drug discovery process involving identification of an endogenous ligand specific for an endogenous receptor, screening of candidate compounds against the receptor for determination of those which interfere and/or compete with the ligand-receptor interaction, and assessing the efficacy of the compound for affecting at least one second messenger pathway associated with the activated receptor.

INHIBIT or INHIBITING, in relationship to the term “response” shall mean that a response is decreased or prevented in the presence of a compound as opposed to in the absence of the compound.

INVERSE AGONISTS shall mean materials (e.g., ligand, candidate compound) which bind to either the endogenous form of the receptor or to the constitutively activated form of the receptor, and which inhibit the baseline intracellular response initiated by the active form of the receptor below the normal base level of activity which is observed in the absence of agonists or partial agonists, or decrease GTP binding to membranes. Preferably, the baseline intracellular response is inhibited in the presence of the inverse agonist by at least 30%, more preferably by at least 50%, and most preferably by at least 75%, as compared with the baseline response in the absence of the inverse agonist.

KNOWN RECEPTOR shall mean an endogenous receptor for which the endogenous ligand specific for that receptor has been identified.

LIGAND shall mean an endogenous, naturally occurring molecule specific for an endogenous, naturally occurring receptor.

MUTANT or MUTATION in reference to an endogenous receptor's nucleic acid and/or amino acid sequence shall mean a specified change or changes to such endogenous sequences such that a mutated form of an endogenous, non-constitutively activated receptor evidences constitutive activation of the receptor. In terms of equivalents to specific sequences, a subsequent mutated form of a human receptor is considered to be equivalent to a first mutation of the human receptor if (a) the level of constitutive activation of the subsequent mutated form of a human receptor is substantially the same as that evidenced by the first mutation of the receptor; and (b) the percent sequence (amino acid and/or nucleic acid) homology between the subsequent mutated form of the receptor and the first mutation of the receptor is at least about 80%, more preferably at least about 90% and most preferably at least 95%. Ideally, and owing to the fact that the most preferred cassettes disclosed herein for achieving constitutive activation includes a single amino acid and/or codon change between the endogenous and the non-endogenous forms of the GPCR, the percent sequence homology should be at least 98%.

NON-ORPHAN RECEPTOR shall mean an endogenous naturally occurring molecule specific for an endogenous naturally occurring ligand wherein the binding of a ligand to a receptor activates an intracellular signaling pathway.

ORPHAN RECEPTOR shall mean an endogenous receptor for which the endogenous ligand specific for that receptor has not been identified or is not known.

PHARMACEUTICAL COMPOSITION shall mean a composition comprising at least one active ingredient, whereby the composition is amenable to investigation for a specified, efficacious outcome in a mammal (for example, and not limitation, a human). Those of ordinary skill in the art will understand and appreciate the techniques appropriate for determining whether an active ingredient has a desired efficacious outcome based upon the needs of the artisan.

PLASMID shall mean the combination of a Vector and cDNA. Generally, a Plasmid is introduced into a Host Cell for the purposes of replication and/or expression of the cDNA as a protein.

STIMULATE or STIMULATING, in relationship to the term “response” shall mean that a response is increased in the presence of a compound as opposed to in the absence of the compound.

VECTOR in reference to cDNA shall mean a circular DNA capable of incorporating at least one cDNA and capable of incorporation into a Host Cell.

The order of the following sections is set forth for presentational efficiency and is not intended, nor should be construed, as a limitation on the disclosure or the claims to follow.

A. Introduction

The traditional study of receptors has always proceeded from the a priori assumption (historically based) that the endogenous ligand must first be identified before discovery could proceed to find antagonists and other molecules that could affect the receptor. Even in cases where an antagonist might have been known first, the search immediately extended to looking for the endogenous ligand. This mode of thinking has persisted in receptor research even after the discovery of constitutively activated receptors. What has not been heretofore recognized is that it is the active state of the receptor that is most useful for discovering agonists, partial agonists, and inverse agonists of the receptor. For those diseases which result from an overly active receptor or an under-active receptor, what is desired in a therapeutic drug is a compound which acts to diminish the active state of a receptor or enhance the activity of the receptor, respectively, not necessarily a drug which is an antagonist to the endogenous ligand. This is because a compound that reduces or enhances the activity of the active receptor state need not bind at the same site as the endogenous ligand. Thus, as taught by a method of this invention, any search for therapeutic compounds should start by screening compounds against the ligand-independent active state.

B. Identification of Human GPCRs

The efforts of the Human Genome project has led to the identification of a plethora of information regarding nucleic acid sequences located within the human genome; it has been the case in this endeavor that genetic sequence information has been made available without an understanding or recognition as to whether or not any particular genomic sequence does or may contain open-reading frame information that translate human proteins. Several methods of identifying nucleic acid sequences within the human genome are within the purview of those having ordinary skill in the art. For example, and not limitation, a variety of human GPCRs, disclosed herein, were discovered by reviewing the GenBank™ database, while other GPCRs were discovered by utilizing a nucleic acid sequence of a GPCR, previously sequenced, to conduct a BLAST™ search of the EST database. Table B, below, lists several endogenous GPCRs that we have discovered, along with a GPCR's respective homologous receptor.

TABLE B

| Disclosed | | Open | Per Cent | Reference To |
|---|---|---|---|---|
| Human | Accession | Reading | Homology | Homologous |
| Orphan | Number | Frame | To Designated | GPCR |
| GPCRs | Identified | (Base Pairs) | GPCR | (Accession No.) |
| hARE-3 | AL033379 | 1,260 bp | 52.3% LPA-R | U92642 |
| hARE-4 | AC006087 | 1,119 bp | 36% P2Y5 | AF000546 |
| hARE-5 | AC006255 | 1,104 bp | 32% Oryzias | D43633 |
| | | | latipes | |
| hGPR27 | AA775870 | 1,128 bp | | |
| hARE-1 | A1090920 | 999 bp | 43% | D13626 |
| | | | KIAA0001 | |
| hARE-2 | AA359504 | 1,122 bp | 53% GPR27 | |
| hPPR1 | 1167224 | 1,053 bp | 39% EBI1 | L31581 |
| hG2A | AA754702 | 1,113 bp | 31% GPR4 | L36148 |
| hRUP3 | AL035423 | 1,005 bp | 30% | 2133653 |
| | | | Drosophila | |
| | | | melanogaster | |
| hRUP4 | A1307658 | 1,296 bp | 32% pNPGPR | NP_004876 |
| | | | 28% and 29% | AAC41276 |
| | | | Zebra fish Ya | and |
| | | | and Yb, | AAB94616 |
| | | | respectively | |
| hRUP5 | AC005849 | 1,413 bp | 25% DEZ | Q99788 |
| | | | 23% FMLPR | P21462 |
| hRUP6 | AC005871 | 1,245 bp | 48% GPR66 | NP_006047 |
| hRUP7 | AC007922 | 1,173 bp | 43% H3R | AF140538 |
| hCHN3 | EST 36581 | 1,113 bp | 53% GPR27 | |
| hCHN4 | AA804531 | 1,077 bp | 32% thrombin | 4503637 |
| hCHN6 | EST 2134670 | 1,503 bp | 36% edg-1 | NP_001391 |
| hCHN8 | EST 764455 | 1,029 bp | 47% | D13626 |
| | | | KIAA0001 | |
| hCHN9 | EST 1541536 | 1,077 bp | 41% LTB4R | NM_000752 |
| hCHN10 | EST 1365839 | 1,055bp | 35% P2Y | NM_002563 |

Receptor homology is useful in terms of gaining an appreciation of a role of the receptors within the human body. As the patent document progresses, we will disclose techniques for mutating these receptors to establish non-endogenous, constitutively activated versions of these receptors.

The techniques disclosed herein have also been applied to other human, orphan GPCRs known to the art, as will be apparent as the patent document progresses.

C. Receptor Screening

Screening candidate compounds against a non-endogenous, constitutively activated version of the human GPCRs disclosed herein allows for the direct identification of candidate compounds which act at this cell surface receptor, without requiring use of the receptor's endogenous ligand. By determining areas within the body where the endogenous version of human GPCRs disclosed herein is expressed and/or over-expressed, it is possible to determine related disease/disorder states which are associated with the expression and/or over-expression of the receptor; such an approach is disclosed in this patent document.

With respect to creation of a mutation that may evidence constitutive activation of the human GPCR disclosed herein is based upon the distance from the proline residue at which is presumed to be located within TM6 of the GPCR; this algorithmic technique is disclosed in co-pending and commonly assigned patent document U.S. Ser. No. 09/170,496, incorporated herein by reference. The algorithmic technique is not predicated upon traditional sequence “alignment” but rather a specified distance from the aforementioned TM6 proline residue. By mutating the amino acid residue located 16 amino acid residues from this residue (presumably located in the IC3 region of the receptor) to, most preferably, a lysine residue, such activation may be obtained. Other amino acid residues may be useful in the mutation at this position to achieve this objective.

D. Disease/Disorder Identification and/or Selection

As will be set forth in greater detail below, most preferably inverse agonists to the non-endogenous, constitutively activated GPCR can be identified by the methodologies of this invention. Such inverse agonists are ideal candidates as lead compounds in drug discovery programs for treating diseases related to this receptor. Because of the ability to directly identify inverse agonists to the GPCR, thereby allowing for the development of pharmaceutical compositions, a search for diseases and disorders associated with the GPCR is relevant. For example, scanning both diseased and normal tissue samples for the presence of the GPCR now becomes more than an academic exercise or one which might be pursued along the path of identifying an endogenous ligand to the specific GPCR. Tissue scans can be conducted across a broad range of healthy and diseased tissues. Such tissue scans provide a preferred first step in associating a specific receptor with a disease and/or disorder. See, for example, co-pending application (docket number ARE-0050) for exemplary dot-blot and RT-PCR results of several of the GPCRs disclosed herein.

Preferably, the DNA sequence of the human GPCR is used to make a probe for (a) dot-blot analysis against tissue-mRNA, and/or (b) RT-PCR identification of the expression of the receptor in tissue samples. The presence of a receptor in a tissue source, or a diseased tissue, or the presence of the receptor at elevated concentrations in diseased tissue compared to a normal tissue, can be preferably utilized to identify a correlation with a treatment regimen, including but not limited to, a disease associated with that disease. Receptors can equally well be localized to regions of organs by this technique. Based on the known functions of the specific tissues to which the receptor is localized, the putative functional role of the receptor can be deduced.

E. Screening of Candidate Compounds

1. Generic GPCR screening assay techniques

When a G protein receptor becomes constitutively active, it binds to a G protein (e.g., Gq, Gs, Gi, Gz, Go) and stimulates the binding of GTP to the G protein. The G protein then acts as a GTPase and slowly hydrolyzes the GTP to GDP, whereby the receptor, under normal conditions, becomes deactivated. However, constitutively activated receptors continue to exchange GDP to GTP. A non-hydrolyzable analog of GTP, [³⁵S]GTPγS, can be used to monitor enhanced binding to membranes which express constitutively activated receptors. It is reported that [³⁵S]GTPγS can be used to monitor G protein coupling to membranes in the absence and presence of ligand. An example of this monitoring, among other examples well-known and available to those in the art, was reported by Traynor and Nahorski in 1995. The preferred use of this assay system is for initial screening of candidate compounds because the system is generically applicable to all G protein-coupled receptors regardless of the particular G protein that interacts with the intracellular domain of the receptor.

2. Specific GPCR Screening Assay Techniques

Once candidate compounds are identified using the “generic” G protein-coupled receptor assay (i.e., an assay to select compounds that are agonists, partial agonists, or inverse agonists), further screening to confirm that the compounds have interacted at the receptor site is preferred. For example, a compound identified by the “generic” assay may not bind to the receptor, but may instead merely “uncouple” the G protein from the intracellular domain.

a. Gs, Gz and Gi.

Gs stimulates the enzyme adenylyl cyclase. Gi (and Gz and Go), on the other hand, inhibit this enzyme. Adenylyl cyclase catalyzes the conversion of ATP to cAMP; thus, constitutively activated GPCRs that couple the Gs protein are associated with increased cellular levels of cAMP. On the other hand, constitutively activated GPCRs that couple Gi (or Gz, Go) protein are associated with decreased cellular levels of cAMP. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Thus, assays that detect cAMP can be utilized to determine if a candidate compound is, e.g., an inverse agonist to the receptor (i.e., such a compound would decrease the levels of cAMP). A variety of approaches known in the art for measuring cAMP can be utilized; a most preferred approach relies upon the use of anti-cAMP antibodies in an ELISA-based format. Another type of assay that can be utilized is a whole cell second messenger reporter system assay. Promoters on genes drive the expression of the proteins that a particular gene encodes. Cyclic AMP drives gene expression by promoting the binding of a cAMP-responsive DNA binding protein or transcription factor (CREB) that then binds to the promoter at specific sites called cAMP response elements and drives the expression of the gene. Reporter systems can be constructed which have a promoter containing multiple cAMP response elements before the reporter gene, e.g., β-galactosidase or luciferase. Thus, a constitutively activated Gs-linked receptor causes the accumulation of cAMP that then activates the gene and expression of the reporter protein. The reporter protein such as β-galactosidase or luciferase can then be detected using standard biochemical assays (Chen et al. 1995).

b. Go and Gq.

Gq and Go are associated with activation of the enzyme phospholipase C, which in turn hydrolyzes the phospholipid PIP2, releasing two intracellular messengers: diacycloglycerol (DAG) and inistol 1,4,5-triphoisphate (IP3). Increased accumulation of IP₃ is associated with activation of Gq- and Go-associated receptors. See, generally, “Indirect Mechanisms of Synaptic Transmission,” Chpt. 8, From Neuron To Brain (3^{rd} Ed.) Nichols, J. G. et al eds. Sinauer Associates, Inc. (1992). Assays that detect IP3 accumulation can be utilized to determine if a candidate compound is, e.g., an inverse agonist to a Gq- or Go-associated receptor (i.e., such a compound would decrease the levels of IP₃). Gq-associated receptors can also been examined using an AP1 reporter assay in that Gq-dependent phospholipase C causes activation of genes containing AP1 elements; thus, activated Gq-associated receptors will evidence an increase in the expression of such genes, whereby inverse agonists thereto will evidence a decrease in such expression, and agonists will evidence an increase in such expression. Commercially available assays for such detection are available.

3. GPCR Fusion Protein

The use of an endogenous, constitutively activate orphan GPCR or a non-endogenous, constitutively activated orphan GPCR, for use in screening of candidate compounds for the direct identification of inverse agonists, agonists and partial agonists provide an interesting screening challenge in that, by definition, the receptor is active even in the absence of an endogenous ligand bound thereto. Thus, in order to differentiate between, e.g., the non-endogenous receptor in the presence of a candidate compound and the non-endogenous receptor in the absence of that compound, with an aim of such a differentiation to allow for an understanding as to whether such compound may be an inverse agonist, agonist, partial agonist or have no affect on such a receptor, it is preferred that an approach be utilized that can enhance such differentiation. A preferred approach is the use of a GPCR Fusion Protein.

Generally, once it is determined that a non-endogenous orphan GPCR has been constitutively activated using the assay techniques set forth above (as well as others), it is possible to determine the predominant G protein that couples with the endogenous GPCR. Coupling of the G protein to the GPCR provides a signaling pathway that can be assessed. Because it is most preferred that screening take place by use of a mammalian expression system, such a system will be expected to have endogenous G protein therein. Thus, by definition, in such a system, the non-endogenous, constitutively activated orphan GPCR will continuously signal. In this regard, it is preferred that this signal be enhanced such that in the presence of, e.g., an inverse agonist to the receptor, it is more likely that it will be able to more readily differentiate, particularly in the context of screening, between the receptor when it is contacted with the inverse agonist.

The GPCR Fusion Protein is intended to enhance the efficacy of G protein coupling with the non-endogenous GPCR. The GPCR Fusion Protein is preferred for screening with a non-endogenous, constitutively activated GPCR because such an approach increases the signal that is most preferably utilized in such screening techniques. This is important in facilitating a significant “signal to noise” ratio; such a significant ratio is import preferred for the screening of candidate compounds as disclosed herein.

The construction of a construct useful for expression of a GPCR Fusion Protein is within the purview of those having ordinary skill in the art. Commercially available expression vectors and systems offer a variety of approaches that can fit the particular needs of an investigator. The criteria of importance for such a GPCR Fusion Protein construct is that the endogenous GPCR sequence and the G protein sequence both be in-frame (preferably, the sequence for the endogenous GPCR is upstream of the G protein sequence) and that the “stop” codon of the GPCR must be deleted or replaced such that upon expression of the GPCR, the G protein can also be expressed. The GPCR can be linked directly to the G protein, or there can be spacer residues between the two (preferably, no more than about 12,although this number can be readily ascertained by one of ordinary skill in the art). We have a preference (based upon convenience) of use of a spacer in that some restriction sites that are not used will, effectively, upon expression, become a spacer. Most preferably, the G protein that couples to the non-endogenous GPCR will have been identified prior to the creation of the GPCR Fusion Protein construct. Because there are only a few G proteins that have been identified, it is preferred that a construct comprising the sequence of the G protein (i.e., a universal G protein construct) be available for insertion of an endogenous GPCR sequence therein; this provides for efficiency in the context of large-scale screening of a variety of different endogenous GPCRs having different sequences.

As noted above, constitutively activated GPCRs that couple to Gi, Gz and Go are expected to inhibit the formation of cAMP making assays based upon these types of GPCRs challenging (i.e., the cAMP signal decreases upon activation thus making the direct identification of, e.g, inverse agonists (which would further decrease this signal), interesting). As will be disclosed herein, we have ascertained that for these types of receptors, it is possible to create a GPCR Fusion Protein that is not based upon the endogenous GPCR's endogenous G protein, in an effort to establish a viable cyclase-based assay. Thus, for example, a Gz coupled receptor such as H9, a GPCR Fusion Protein can be established that utilizes a Gs fusion protein—we believe that such a fusion construct, upon expression, “drives” or “forces” the non-endogenous GPCR to couple with, e.g., Gs rather than the “natural” Gz protein, such that a cyclase-based assay can be established. Thus, for Gi, Gz and Go coupled receptors, we prefer that that when a GPCR Fusion Protein is used and the assay is based upon detection of adenyl cyclase activity, that the fusion construct be established with Gs (or an equivalent G protein that stimulates the formation of the enzyme adenylyl cyclase).

F. Medicinal Chemistry

Generally, but not always, direct identification of candidate compounds is preferably conducted in conjunction with compounds generated via combinatorial chemistry techniques, whereby thousands of compounds are randomly prepared for such analysis. Generally, the results of such screening will be compounds having unique core structures; thereafter, these compounds are preferably subjected to additional chemical modification around a preferred core structure(s) to further enhance the medicinal properties thereof. Such techniques are known to those in the art and will not be addressed in detail in this patent document.

G. Pharmaceutical Compositions

Candidate compounds selected for further development can be formulated into pharmaceutical compositions using techniques well known to those in the art. Suitable pharmaceutically-acceptable carriers are available to those in the art; for example, see Remington's Pharmaceutical Sciences, 16^{th} Edition, 1980, Mack Publishing Co., (Oslo et al., eds.)

H. Other Utility

Although a preferred use of the non-endogenous versions the human GPCRs disclosed herein may be for the direct identification of candidate compounds as inverse agonists, agonists or partial agonists (preferably for use as pharmaceutical agents), these versions of human GPCRs can also be utilized in research settings. For example, in vitro and in vivo systems incorporating GPCRs can be utilized to further elucidate and understand the roles these receptors play in the human condition, both normal and diseased, as well as understanding the role of constitutive activation as it applies to understanding the signaling cascade. The value in non-endogenous human GPCRs is that their utility as a research tool is enhanced in that, because of their unique features, non-endogenous human GPCRs can be used to understand the role of these receptors in the human body before the endogenous ligand therefor is identified. Other uses of the disclosed receptors will become apparent to those in the art based upon, inter alia, a review of this patent document.

### EXAMPLES

The following examples are presented for purposes of elucidation, and not limitation, of the present invention. While specific nucleic acid and amino acid sequences are disclosed herein, those of ordinary skill in the art are credited with the ability to make minor modifications to these sequences while achieving the same or substantially similar results reported below. The traditional approach to application or understanding of sequence cassettes from one sequence to another (e.g. from rat receptor to human receptor or from human receptor A to human receptor B) is generally predicated upon sequence alignment techniques whereby the sequences are aligned in an effort to determine areas of commonality. The mutational approach disclosed herein does not rely upon this approach but is instead based upon an algorithmic approach and a positional distance from a conserved proline residue located within the TM6 region of human GPCRs. Once this approach is secured, those in the art are credited with the ability to make minor modifications thereto to achieve substantially the same results (i.e., constitutive activation) disclosed herein. Such modified approaches are considered within the purview of this disclosure

### Example 1

Endogenous Human GPCRs

1. Identification of Human GPCRs

Certain of the disclosed endogenous human GPCRs were identified based upon a review of the GenBank™ database information. While searching the database, the following cDNA clones were identified as evidenced below (Table C).

TABLE C

| Disclosed | | | Open | Nucleic | Amino |
|---|---|---|---|---|---|
| Human | | Complete DNA | Reading | Acid | Acid |
| Orphan | Accession | Sequence | Frame | SEQ. | SEQ. |
| GPCRs | Number | (Base Pairs) | (Base Pairs) | ID.NO. | ID.NO. |
| hARE-3 | AL033379 | 111,389 bp | 1,260 bp | 1 | 2 |
| hARE-4 | AC006087 | 226,925 bp | 1,119 bp | 3 | 4 |
| hARE-5 | AC006255 | 127,605 bp | 1,104 bp | 5 | 6 |
| hRUP3 | AL035423 | 140,094 bp | 1,005 bp | 7 | 8 |
| hRUP5 | AC005849 | 169,144 bp | 1,413 bp | 9 | 10 |
| hRUP6 | AC005871 | 218,807 bp | 1,245 bp | 11 | 12 |
| hRUP7 | AC007922 | 158,858 bp | 1,173 bp | 13 | 14 |

Other disclosed endogenous human GPCRs were identified by conducting a BLAST™ search of EST database (dbest) using the following EST clones as query sequences. The following EST clones identified were then used as a probe to screen a human genomic library (Table D).

TABLE D

| Disclosed | | | Open | | |
|---|---|---|---|---|---|
| Human | | EST Clone/ | Reading | | |
| Orphan | Query | Accession No. | Frame | Nucleic Acid | Amino Acid |
| GPCRs | (Sequence) | Identified | (Base Pairs) | SEQ.ID.NO. | SEQ.ID.NO. |
| hGPCR27 | Mouse | AA775870 | 1,125 bp | 17 | 18 |
| | GPCR27 | | | | |
| hARE-1 | TDAG | 1689643 | 999 bp | 19 | 20 |
| | | AI090920 | | | |
| hARE-2 | GPCR27 | 68530 | 1,122 bp | 21 | 22 |
| | | AA359504 | | | |
| hPPR1 | Bovine | 238667 | 1,053 bp | 23 | 24 |
| | PPR1 | H67224 | | | |
| hG2A | Mouse | See Example 2(a), | 1,113 bp | 25 | 26 |
| | 1179426 | below | | | |
| hCHN3 | N.A. | EST 36581 | 1,113 bp | 27 | 28 |
| | | (full length) | | | |
| hCHN4 | TDAG | 1184934 | 1,077 bp | 29 | 30 |
| | | AA804531 | | | |
| hCHN6 | N.A. | EST 2134670 | 1,503 bp | 31 | 32 |
| | | (full length) | | | |
| hCHN8 | KIAA0001 | EST 764455 | 1,029 bp | 33 | 34 |
| hCHN9 | 1365839 | EST 1541536 | 1,077 bp | 35 | 36 |
| hCHN10 | Mouse EST | Human 1365839 | 1,005 bp | 37 | 38 |
| | 1365839 | | | | |
| hRUP4 | N.A. | AI307658 | 1,296 bp | 39 | 40 |

N.A. = “not applicable”.

2. Full Length Cloning

a. Human G2A

Mouse EST clone 1179426 was used to obtain a human genomic clone containing all but three amino acid G2A coding sequences. The 5′ of this coding sequence was obtained by using 5′ RACE, and the template for PCR was Clontech's Human Spleen Marathon-Ready™ cDNA. The disclosed human G2A was amplified by PCR using the G2A cDNA specific primers for the first and second round PCR as shown in SEQ.ID.NO.: 41 and SEQ.ID.NO.:42 as follows:

| | |
|---|---|
| 5′-CTGTGTACAGCAGTTCGCAGAGTG-3′ | (SEQ.ID.NO.:41; 1^{st} round PCR) |
| 5′-GAGTGCCAGGCAGAGCAGGTAGAC-3′ | (SEQ.ID.NO.:42; second round PCR). |

PCR was performed using Advantage GC Polymerase Kit (Clontech; manufacturing instructions will be followed), at 94° C. for 30 sec followed by 5 cycles of 94° C. for 5 sec and 72° C. for 4 min; and 30 cycles of 94° C. for 5 sec and 70° for 4 min. An approximate 1.3 Kb PCR fragment was purified from agarose gel, digested with Hind III and Xba I and cloned into the expression vector pRC/CMV2 (Invitrogen). The cloned-insert was sequenced using the T7 Sequenase™ kit (USB Amersham; manufacturer instructions followed) and the sequence was compared with the presented sequence. Expression of the human G2A was detected by probing an RNA dot blot (Clontech; manufacturer instructions followed) with the P³²-labeled fragment.

b. CHN9

Sequencing of the EST clone 1541536 showed CHN9 to be a partial cDNA clone having only an initiation codon; i.e., the termination codon was missing. When CHN9 was used to blast against data base (nr), the 3′ sequence of CHN9 was 100% homologous to the 5′ untranslated region of the leukotriene B4 receptor cDNA, which contained a termination codon in the frame with CHN9 coding sequence. To determine whether the 5′ untranslated region of LTB4R cDNA was the 3′ sequence of CHN9, PCR was performed using primers based upon the 5′ sequence flanking the initiation codon found in CHN9 and the 3′ sequence around the termination codon found in the LTB4R 5′ untranslated region. The 5′ primer sequence utilized was as follows:

| | |
|---|---|
| 5′-CCCGAATTCCTGCTTGCTCCCAGCTTGGCCC-3′ | (SEQ.ID.NO.:43; sense) and |
| 5′-TGTGGATCCTGCTGTCAAAGGTCCCATTCCGG-3′ | (SEQ.ID.NO.:44; antisense). |

PCR was performed using thymus cDNA as a template and rTth polymerase (Perkin Elmer) with the buffer system provided by the manufacturer, 0.25 uM of each primer, and 0.2 mM of each 4 nucleotides. The cycle condition was 30 cycles of 94° C. for 1 min, 65° C. for 1 min and 72° C. for 1 min and 10 sec. A 1.1 kb fragment consistent with the predicted size was obtained from PCR. This PCR fragment was subcloned into pCMV (see below) and sequenced (see, SEQ.ID.NO.: 35).

c. RUP4

The full length RUP4 was cloned by RT-PCR with human brain cDNA (Clontech) as templates:

| | |
|---|---|
| 5′-TCACAATGCTAGGTGTGGTC-3′ | (SEQ.ID.NO.:45; sense) and |
| 5′-TGCATAGACAATGGGATTACAG-3′ | (SEQ.ID.NO.:46; antisense). |

PCR was performed using TaqPlus Precision™ polymerase (Stratagene; manufacturing instructions followed) by the following cycles: 94° C. for 2 min; 94° C. 30 sec; 55° C. for 30 sec, 72° C. for 45 sec, and 72° C. for 10 min. Cycles 2 through 4 were repeated 30 times.

The PCR products were separated on a 1% agarose gel and a 500 bp PCR fragment was isolated and cloned into the pCRII-TOPO™ vector (Invitrogen) and sequenced using the T7 DNA Sequenase™ kit (Amsham) and the SP6/T7 primers (Stratagene). Sequence analysis revealed that the PCR fragment was indeed an alternatively spliced form of AI3 0765 8 having a continuous open reading frame with similarity to other GPCRs. The completed sequence of this PCR fragment was as follows:

| | |
|---|---|
| 5′-TCACAATGCTAGGTGTGGTCTGGCTGGTGGCAGTCATCGTAGGATCACCCATGTGGCAC | (SEQ.ID.NO.:47) |
| GTGCAACAACTTGAGATCAAATATGACTTCCTATATGAAAAGGAACACATCTGCTGCTTAAGA | |
| GTGGACCAGCCCTGTGCACCAGAAGATCTACACCACCTTCATCCTTGTCATCCTCTTCCTCCTGC | |
| CTCTTATGGTGATGCTTATTCTGTACGTAAAATTGGTTATGAACTTTGGATAAAGAAAAGAGTT | |
| GGGGATGGTTCAGTGCTTCGAACTATTCATGGAAAAGAAATGTCCAAAATAGCCAGGAAGAAG | |
| AAACGAGCTGTCATTATGATGGTGACAGTGGTGGCTCTCTTTGCTGTGCTGGGCACCATTCC | |
| ATGTTGTCCATATGATGATTGAATACAGTAATTTTGAAAAGGAATATGATGATGTCACAATCAA | |
| GATGATTTTTGCTATCGTGCAAATTATTGGATTTTCCAACTCCATCTGTAATCCCATTGTCTATGCA-3′ | |

Based on the above sequence, two sense oligonucleotide primer sets:

| | |
|---|---|
| 5′-CTGCTTAGAAGAGTGGACCAG-3′ | (SEQ.ID.NO.:48; |
| | oligo 1), |
| 5′-CTGTGCACCAGAAGATCTACAC-3′ | (SEQ.IDNO.:49; |
| | oligo 2) |

and two antisense oligonucleotide primer sets:

| | |
|---|---|
| 5′-CAAGGATGAAGGTGGTGTAGA-3′ | (SEQ.ID.NO.:50; |
| | oligo 3) |
| 5′-GTGTAGATGTTCTGGTGCAGAGG-3′ | (SEQ.ID.NO.:51; |
| | oligo 4) |

were used for 3′- and 5′-RACE PCR with a human brain Marathon-Ready™ cDNA (Clontech, Cat# 7400-1) as template, according to manufacture's instructions. DNA fragments generated by the RACE PCR were cloned into the pCRII-TOPO™ vector (Invitrogen) and sequenced using the SP6/T7 primers (Stratagene) and some internal primers. The 3′ RACE product contained a poly(A) tail and a completed open reading frame ending at a TAA stop codon. The 5′ RACE product contained an incomplete 5′ end; i.e., the ATG initiation codon was not present.

Based on the new 5′ sequence, oligo 3 and the following primer:

5′-GCAATGCAGGTCATAGTGAGC -3′ (SEQ.ID.NO.: 52; oligo 5)

were used for the second round of 5′ race PCR and the PCR products were analyzed as above. A third round of 5′ race PCR was carried out utilizing antisense primers:

| | |
|---|---|
| 5′-TGGAGCATGGTGACGGGAATGCAGAAG-3′ | (SEQ.ID.NO.:53; oligo 6) and |
| 5′-GTGATGAGCAGGTCACTGAGCGCCAAG-3′ | (SEQ.ID.NO.:54; oligo7). |

The sequence of the 5′ RACE PCR products revealed the presence of the initiation codon ATG, and further round of 5′ race PCR did not generate any more 5′ sequence. The completed 5′ sequence was confirmed by RT-PCR using sense primer

5′-GCAATGCAGGCGCTTAACATTAC-3′ (SEQ.ID.NO.: 55; oligo 8)

and oligo 4 as primers and sequence analysis of the 650 bp PCR product generated from human brain and heart cDNA templates (Clontech, Cat# 7404-1). The completed 3′ sequence was confirmed by RT-PCR using oligo 2 and the following antisense primer:

5′-TTGGGTTACAATCTGAAGGGCA-3′ (SEQ.ID.NO.:56; oligo 9)

and sequence analysis of the 670 bp PCR product generated from human brain and heart cDNA templates. (Clontech, Cat# 7404-1).

d. RUP5

The full length RUP5 was cloned by RT-PCR using a sense primer upstream from ATG, the initiation codon (SEQ.ID.NO.:57), and an antisense primer containing TCA as the stop codon (SEQ.ID.NO.:58), which had the following sequences:

| | |
|---|---|
| 5′-ACTCCGTGTCCAGCAGGACTCTG-3′ | (SEQ.ID.NO.:57) |
| 5′-TGCGTGTTCCTGGACCCTCACGTG-3′ | (SEQ.ID.NO.:58) |

and human peripheral leukocyte cDNA (Clontech) as a template. Advantage™ cDNA polymerase (Clontech) was used for the amplification in a 50 ul reaction by the following cycle with step 2 through step 4 repeated 30 times: 94° C. for 30 sec; 94° for 15 sec; 69° for 40 sec; 72° C. for 3 min; and 72° C. fro 6 min. A 1.4 kb PCR fragment was isolated and cloned with the pCRII-TOPO™ vector (Invitrogen) and completely sequenced using the T7 DNA Sequenase™ kit (Amsham). See, SEQ.ID.NO.: 9.

e. RUP6

The full length RUP6 was cloned by RT-PCR using primers:

| | |
|---|---|
| 5′-CAGGCCTTGGATTTTAATGTCAGGGATGG-3′ | (SEQ.ID. |
| | NO.:59) and |
| 5′-GGAGAGTCAGCTCTGAAAGAATTCAGG-3′ | (SEQ.ID. |
| | NO.:60); |

and human thymus Marathon-Ready™ cDNA (Clontech) as a template. Advantage cDNA polymerase (Clontech, according to manufacturer's instructions) was used for the amplification in a 50 ul reaction by the following cycle: 94° C. for 30 sec; 94° C. for 5 sec; 66° C. for 40 sec; 72° C. for 2.5 sec and 72° C. for 7 min. Cycles 2 through 4 were repeated 30 times. A 1.3 Kb PCR fragment was isolated and cloned into the pCRII-TOPO™ vector (Invitrogen) and completely sequenced (see, SEQ.ID.NO.: 11) using the ABI Big Dye Terminator™ kit (P.E. Biosystem).

f. RUP7

The full length RUP7 was cloned by RT-PCR using primers:

| | |
|---|---|
| 5′-TGATGTGATGCCAGATACTAATAGCAC-3′ | (SEQ.ID.NO.:61; sense) and |
| 5′-CCTGATTCATTTAGGTGAGATTGAGAC-3′ | (SEQ.ID.NO.:62; antisense) |

and human peripheral leukocyte cDNA (Clontech) as a template. Advantage™ cDNA polymerase (Clontech) was used for the amplification in a 50 ul reaction by the following cycle with step 2 to step 4 repeated 30 times: 94° C. for 2 minutes; 94° C. for 15 seconds; 60° C. for 20 seconds; 72° C. for 2 minutes; 72° C. for 10 minutes. A 1.25 Kb PCR fragment was isolated and cloned into the pCRII-TOPO™ vector (Invitrogen) and completely sequenced using the ABI Big Dye Terminator™ kit (P.E. Biosystem). See, SEQ.ID.NO.: 13.

3. Angiotensin II Type 1 Receptor (“AT1”)

The endogenous human angiotensin II type 1 receptor (“AT1”) was obtained by PCR using genomic DNA as template and rTth polymerase (Perkin Elmer) with the buffer system provided by the manufacturer, 0.25 μM of each primer, and 0.2 mM of each 4 nucleotides. The cycle condition was 30 cycles of 94° C. for 1 min, 55° C. for 1 min and 72° C. for 1.5 min. The 5′ PCR primer contains a HindIII site with the sequence:

5′-CCCAAGCTTCCCCAGGTGTATTTGAT-3′ (SEQ.ID.NO.: 63)

and the 3′ primer contains a BamHI site with the following sequence:

5′-GTTGGATCCACATAATGCATTTCTC-3′ (SEQ.ID.NO.: 64).

The resulting 1.3 kb PCR fragment was digested with HindIII and BamHI and cloned into HindIII-BamHI site of pCMV expression vector. The cDNA clone was fully sequenced. Nucleic acid (SEQ.ID.NO.: 65) and amino acid (SEQ.ID.NO.: 66) sequences for human AT1 were thereafter determined and verified.

4. GPR38

To obtain GPR38, PCR was performed by combining two PCR fragments, using human genomic cDNA as template and rTth poymerase (Perkin Elmer) with the buffer system provided by the manufacturer, 0.25 uM of each primer, and 0.2 mM of each 4 nucleotides. The cycle condition for each PCR reaction was 30 cycles of 94° C. for 1 min, 62° C. for 1 min and 72° C. for 2 min.

The first fragment was amplified with the 5′ PCR primer that contained an end site with the following sequence:

5′-ACCATGGGCAGCCCCTGGAACGGCAGC-3′ (SEQ.ID.NO.:67)

and a 3′ primer having the following sequence:

5′-AGAACCACCACCAGCAGGACGCGGACGGTCTGCCGGTGG-3′ (SEQ.ID.NO.:68).

The second PCR fragment was amplified with a 5′ primer having the following sequence:

5′-GTCCGCGTCCTGCTGGTGGTGGTTCTGGCATTTATAATT-3′ (SEQ.ID.NO.: 69)

and a 3′ primer that contained a BamHI site and having the following sequence:

5′-CCTGGATCCTTATCCCATCGTCTTCACGTTAGC-3′ (SEQ.ID.NO.: 70).

The two fragments were used as templates to amplify GPR38, using SEQ.ID.NO.: 67 and SEQ.ID.NO.: 70 as primers (using the above-noted cycle conditions). The resulting 1.44 kb PCR fragment was digested with BamHI and cloned into Blunt-BamHI site of pCMV expression vector.

5. MC4

To obtain MC4, PCR was performed using human genomic cDNA as template and rTth poymerase (Perkin Elmer) with the buffer system provided by the manufacturer, 0.25 uM of each primer, and 0.2 mM of each 4 nucleotides. The cycle condition for each PCR reaction was 30 cycles of 94° C. for 1 min, 54° C. for 1 min and 72° C. for 1.5 min.

The 5′ PCR contained an EcoRI site with the sequence:

5′-CTGGAATTCTCCTGCCAGCATGGTGA-3′ (SEQ.ID.NO.: 71)

and the 3′ primer contained a BamHI site with the sequence:

5′-GCAGGATCCTATATTGCGTGCTCTGTCCCC′-3 (SEQ.ID.NO.: 72).

The 1.0 kb PCR fragment was digest with EcoRI and BamHI and cloned into EcoRI-BamHI site of pCMV expression vector. Nucleic acid (SEQ.ID.NO.: 73) and amino acid (SEQ.ID.NO.: 74) sequences for human MC4 were thereafter determined.

6. CCKB

To obtain CCKB, PCR was performed using human stomach cDNA as template and rTth poymerase (Perkin Elmer) with the buffer system provided by the manufacturer, 0.25 uM of each primer, and 0.2 mM of each 4 nucleotides. The cycle condition for each PCR reaction was 30 cycles of 94° C. for 1 min, 65° C. for 1 min and 72° C. for 1 min and 30 sec. The 5′ PCR contained a HindIII site with the sequence:

5′-CCGAAGCTTCGAGCTGAGTAAGGCGGCGGGCT-3′ (SEQ.ID.NO.: 75)

and the 3′ primer contained an EcoRI site with the sequence:

5′-GTGGAATTCATTTGCCCTGCCTCAACCCCCA-3 (SEQ.ID.NO.: 76).

The resulting 1.44 kb PCR fragment was digest with HindIII and EcoRI and cloned into HindIII-EcoRI site of pCMV expression vector. Nucleic acid (SEQ.ID.NO.: 77) and amino acid (SEQ.ID.NO.: 78) sequences for human CCKB were thereafter determined.

7. TDAG8

To obtain TDAG8, PCR was performed using genomic DNA as template and rTth polymerase (Perkin Elmer) with the buffer system provided by the manufacturer, 0.25 μM of each primer, and 0.2 mM of each 4 nucleotides. The cycle condition was 30 cycles of 94° C. for 1 min, 56° C. for 1 min and 72° C. for 1 min and 20 sec. The 5′ PCR primer contained a HindIII site with the following sequence:

5′-TGCAAGCTTAAAAAGGAAAAAATGAACAGC-3′ (SEQ.ID.NO.: 79)

and the 3′ primer contained a BamHI site with the following sequence:

5′-TAAGGATCCCTTCCCTTCAAAACATCCTTG -3′ (SEQ.ID.NO.: 80).

The resulting 1.1 kb PCR fragment was digested with HindIII and BamHI and cloned into HindIII-BamHI site of pCMV expression vector. Three resulting clones sequenced contained three potential polymorphisms involving changes of amino acid 43 from Pro to Ala, amino acid 97 from Lys to Asn and amino acid 130 from Ile to Phe. Nucleic acid (SEQ.ID.NO.: 81) and amino acid (SEQ.ID.NO.: 82) sequences for human TDAG8 were thereafter determined.

8. H9

To obtain H9, PCR was performed using pituitary cDNA as template and rTth polymerase (Perkin Elmer) with the buffer system provided by the manufacturer, 0.25 μM of each primer, and 0.2 mM of each 4 nucleotides. The cycle condition was 30 cycles of 94° C. for 1 min, 62° C. for 1 min and 72° C. for 2 min. The 5′ PCR primer contained a HindIII site with the following sequence:

5′-GGAAAGCTTAACGATCCCCAGGAGCAACAT-3′ (SEQ.ID.NO.:15)

and the 3′ primer contained a BamHI site with the following sequence:

5′-CTGGGATCCTACGAGAGCATTTTTCACACAG-3′ (SEQ.ID.NO.:16).

The resulting 1.9 kb PCR fragment was digested with HindIII and BamHI and cloned into HindIII-BamHI site of pCMV expression vector. H9 contained three potential polymorphisms involving changes of amino acid P320S, S493N and amino acid G448A. Nucleic acid (SEQ.ID.NO.: 139) and amino acid (SEQ.ID.NO.: 140) sequences for human H9 were thereafter determined and verified.

### Example 2

Preparation of Non-endogenous, Constitutively Activated GPCRs

Those skilled in the art are credited with the ability to select techniques for mutation of a nucleic acid sequence. Presented below are approaches utilized to create non-endogenous versions of several of the human GPCRs disclosed above. The mutations disclosed below are based upon an algorithmic approach whereby the 16^{th} amino acid (located in the IC3 region of the GPCR) from a conserved proline residue (located in the TM6 region of the GPCR, near the TM6/IC3 interface) is mutated, most preferably to a lysine amino acid residue.

1. Tranformer Site-directed™ Mutagenesis

Preparation of non-endogenous human GPCRs may be accomplished on human GPCRs using Transformer Site-Directed™ Mutagenesis Kit (Clontech) according to the manufacturer instructions. Two mutagenesis primers are utilized, most preferably a lysine mutagenesis oligonucleotide that creates the lysine mutation, and a selection marker oligonucleotide. For convenience, the codon mutation to be incorporated into the human GPCR is also noted, in standard form (Table E):

TABLE E

| Receptor Identifier | Codon Mutation |
|---|---|
| hARE-3 | F313K |
| hARE-4 | V233K |
| hARE-5 | A240K |
| hGPCR14 | L257K |
| hGPCR27 | C283K |
| hARE-1 | E232K |
| hARE-2 | G285K |
| hPPR1 | L239K |
| hG2A | K232A |
| hRUP3 | L224K |
| hRUP5 | A236K |
| hRUP6 | N267K |
| hRUP7 | A302K |
| hCHN4 | V236K |
| hMC4 | A244K |
| hCHN3 | S284K |
| hCHN6 | L352K |
| hCHN8 | N235K |
| hCHN9 | 0223K |
| hCHN10 | L231K |
| hH9 | F236K |

The following GPCRs were mutated according with the above method using the designated sequence primers (Table F).

TABLE F

| | | Lysine Mutagenesis | |
|---|---|---|---|
| | | (SEQ. ID. NO.) | Selection Marker |
| Receptor | Codon | > 5′-3′ orientation, mutation | (SEQ. ID. NO.) |
| Identifier | Mutation | sequence underlined | 5′-3′ orientation |
| hRUP4 | V272K | CAGGAAGAAGAAACGAGC | CACTGTCACCATCATAATG |
| | | TGTCATTATGATGGTGACA | ACAGCTCGTTTCTTCTTCC |
| | | GTG (83) | TG(84) |
| hAT1 | see below | alternative approach; see below | alternative approach; see below |
| hGPR38 | V297K | GGCCACCGGCAGACCAAAC | CTCCTTCGGTCCTCCTATC |
| | | GCGTCCTGCTG (85) | GTTGTCAGAAGT (86) |
| hCCKB | V332K | alternative approach; see below | alternative approach; see below |
| hTDAG8 | I225K | GGAAAAGAAGAGAATCAA | CTCCTTCGGTCCTCCTATC |
| | | AAAACTACTTGTCAGCATC | GTTGTCAGAAGT (88) |
| | | (87) | |
| hH9 | F236K | GCTGAGGTTCGCAATAAAC | CTCCTTCGGTCCTCCTATC |
| | | TAACCATGTTTGTG (143) | GTTGTCAGAAGT (144) |
| hMC4 | A244K | GCCAATATGAAGGGAAAA | CTCCTTCGGTCCTCCTATC |
| | | ATTACCTTGACCATC (137) | GTTGTCAGAAAGT (138) |

The non-endogenous human GPCRs were then sequenced and the derived and verified nucleic acid and amino acid sequences are listed in the accompanying “Sequence Listing” appendix to this patent document, as summarized in Table G below:

TABLE G

| Non Endogenous Human | Nucleic Acid | Amino Acid |
|---|---|---|
| GPCR | Sequence Listing | Sequence Listing |
| hRIJP4 | SEQ.ID.NO.: 127 | SEQ.ID.NO.: 128 |
| (V272K) | | |
| hAT1 | (see alternative approaches | (see alternative approaches, |
| (see alternative approaches | below) | below) |
| below) | | |
| hGPR38 | SEQ.ID.NO.: 129 | SEQ.ID.NO.: 130 |
| (V297K) | | |
| hCCKB | SEQ.ID.NO.: 131 | SEQ.ID.NO.: 132 |
| (V332K) | | |
| HTDAG8 | SEQ.ID.NO.: 133 | SEQ.ID.NO.: 134 |
| (1225K) | | |
| hH9 | SEQ.ID.NO.: 141 | SEQ.ID.NO.: 142 |
| (F236K) | | |
| hMC4 | SEQ.ID.NO.: 135 | SEQ.TD.NO.: 136 |
| (A244K) | | |

2. Alternative Approaches for Creation of Non-endogenous Human GPCRs

a. AT1

1. F239K Mutation

Preparation of a non-endogenous, constitutively activated human AT1 receptor was accomplished by creating an F239K mutation (see, SEQ.ID.NO.:89 for nucleic acid sequence, and SEQ.ID.NO.: 90 for amino acid sequence). Mutagenesis was performed using Transformer Site-Directed Mutagenesis™ Kit (Clontech) according to the to manufacturer's instructions. The two mutagenesis primers were used, a lysine mutagenesis oligonucleotide (SEQ.ID.NO.: 91) and a selection marker oligonucleotide (SEQ.ID.NO.: 92), which had the following sequences:

| | |
|---|---|
| 5′-CCAAGAAATGATGATATTAAAAAGATAATTATGGC-3′ | (SEQ. ID. NO.: 91) |
| 5′-CTCCTTCGGTCCTCCTATCGTTGTCAGAAGT-3′ | (SEQ. ID. NO.: 92), |

respectively.

2. N111A Mutation

Preparation of a non-endogenous human AT1 receptor was also accomplished by creating an N111A mutation (see, SEQ.ID.NO.:93 for nucleic acid sequence, and SEQ.ID.NO.: 94 for amino acid sequence). Two PCR reactions were performed using pfu polymerase (Stratagene) with the buffer system provided by the manufacturer, supplemented with 10% DMSO, 0.25 μM of each primer, and 0.5 mM of each 4 nucleotides. The 5′ PCR sense primer used had the following sequence:

5′-CCCAAGCTTCCCCAGGTGTATTTGAT-3′ (SEQ.ID.NO.: 95)

and the antisense primer had the following sequence:

5′-CCTGCAGGCGAAACTGACTCTGGCTGAAG-3′ (SEQ.ID.NO.: 96).

The resulting 400 bp PCR fragment was digested with HindIII site and subcloned into HindIII-SmaI site of pCMV vector (5′ construct). The 3′ PCR sense primer used had the following sequence:

5′-CTGTACGCTAGTGTGTTTCTACTCACGTGTCTCAGCATTGAT-3′ (SEQ.ID.NO.: 97)

and the antisense primer had the following sequence:

5′-GTTGGATCCACATAATGCATTTTCTC-3′ (SEQ.ID.NO.: 98)

The resulting 880 bp PCR fragment was digested with BamHI and inserted into Pst (blunted by T4 polymerase) and BamHI site of 5′ construct to generated the full length N111A construct. The cycle condition was 25 cycles of 94° C. for 1 min, 60° C. for 1 min and 72° C. for 1 min (5′ PCR) or 1.5 min (3′ PCR).

3. AT2K255IC3 Mutation

Preparation of a non-endogenous, constitutively activated human AT1 was accomplished by creating an AT2K255IC3 “domain swap” mutation (see, SEQ.ID.NO.:99 for nucleic acid sequence, and SEQ.ID.NO.: 100 for amino acid sequence). Restriction sites flanking IC3 of AT1 were generated to facilitate replacement of the IC3 with corresponding IC3 from angiotensin II type 2 receptor (AT2). This was accomplished by performing two PCR reactions. A 5′ PCR fragment (Fragment A) encoded from the 5′ untranslated region to the beginning of IC3 was generated by utilizing SEQ.ID.NO.: 63 as sense primer and the following sequence:

5′-TCCGAATTCCAAAATAACTTGTAAGAATGATCAGAAA-3′ (SEQ. ID.NO.: 101)

as antisense primer. A 3′ PCR fragment (Fragment B) encoding from the end of IC3 to the 3′ untranslated region was generated by using the following sequence:

5′-AGATCTTAAGAAGATAATTATGGCAATTGTGCT-3′ (SEQ.ID.NO.: 102)

as sense primer and SEQ.ID.NO.: 64 as antisense primer. The PCR condition was 30 cycles of 94° C. for 1 min, 55° C. for 1 min and 72° C. for 1.5 min using endogenous AT1 cDNA clone as template and pfu polymerase (Stratagene), with the buffer systems provided by the manufacturer, supplemented with 10% DMSO, 0.25 μM of each primer, and 0.5 mM of each 4 nucleotides. Fragment A (720 bp) was digested with HindIII and EcoRI and subcloned. Fragment B was digested with BamHI and subcloned into pCMV vector with an EcoRI site 5′ to the cloned PCR fragment.

The DNA fragment (Fragment C) encoding IC3 of AT2 with a L255K mutation and containing an EcoRI cohesive end at 5′ and a AflII cohesive end at 3′, was generated by annealing 2 synthetic oligonucleotides having the following sequences:

5′AATTCGAAAACACTTACTGAAGACGAATAGCTATGGGAAGAACAGGAT
AACCCGTGACCAAG-3′
(sense; SEQ.ID.NO.: 103)
5′TTAACTTGGTCACGGGTTATCCTGTTCTTCCCATAGCTATTCGTCTTC
AGTAAGTGTTTTCG-3′
(antisense; SEQ.ID.NO.: 104).

Fragment C was inserted in front of Fragment B through EcoRI and AflII site. The resulting clone was then ligated with the Fragment A through the EcoRI site to generate AT1 with AT2K255IC3.

4. A243+ Mutation

Preparation of a non-endogenous human AT1 receptor was also accomplished by creating an A243+ mutation (see, SEQ.ID.NO.: 105 for nucleic acid sequence, and SEQ.ID.NO.: 106 for amino acid sequence). An A243+ mutation was constructed using the following PCR based strategy: Two PCR reactions was performed using pfu polymerase (Stratagene) with the buffer system provided by the manufacturer supplemented with 10% DMSO, 0.25 μM of each primer, and 0.5 mM of each 4 nucleotides. The 5′ PCR sense primer utilized had the following sequence:

5′-CCCAAGCTTCCCCAGGTGTATTGAT-3′ (SEQ.ID.NO.:107)

and the antisense primer had the following sequence:

5′-AAGCACAATTGCTGCATAATTATCTTAAAAATATCATC-3′ (SEQ.ID.NO.: 108).

The 3′ PCR sense primer utilized had the following sequence:

5′-AAGATAATTATGGCAGCAATTGTGCTTCTTTTTCT=T-3′ (SEQ.ID.NO.: 109)

containing the Ala insertion and antisense primer:

5′-GTTGGATCCACATAATGCATTTCTC-3′ (SEQ.ID.NO.: 110).

The cycle condition was 25 cycles of 94° C. for 1 min, 54° C. for 1 min and 72° C. for 1.5 min. An aliquot of the 5′ and 3′ PCR were then used as co-template to perform secondary PCR using the 5′ PCR sense primer and 3′ PCR antisense primer. The PCR condition was the same as primary PCR except the extention time was 2.5 min. The resulting PCR fragment was digested with HindIII and BamHI and subcloned into pCMV vector. (See, SEQ.ID.NO.: 105)

4. CCKB

Preparation of the non-endogenous, constitutively activated human CCKB receptor was accomplished by creating a V322K mutation (see, SEQ.ID.NO.: 111 for nucleic acid sequence and SEQ.ID.NO.: 112 for amino acid sequence). Mutagenesis was performed by PCR via amplification using the wildtype CCKB from Example 1.

The first PCR fragment (1 kb) was amplified by using SEQ.ID.NO.: 75 and an antisense primer comprising a V322K mutation:

5′-CAGCAGCATGCGCTTCACGCGCTTCTTAGCCCAG-3′ (SEQ.ID.NO.: 113).

The second PCR fragment (0.44 kb) was amplified by using a sense primer comprising the V322K mutation:

5′-AGAAGCGCGTGAAGCGCATGCTGCTGGTGATCGTT-3′ (SEQ.ID.NO.:114) and SEQ.ID.NO.: 76.

The two resulting PCR fragments were then used as template for amplifying CCKB comprising V332K, using SEQ.ID.NO.: 75 and SEQ.ID.NO.: 76 and the above-noted system and conditions. The resulting 1.44 kb PCR fragment containing the V332K mutation was digested with HindIII and EcoRI and cloned into HindIII-EcoRI site of pCMV expression vector. (See, SEQ.ID.NO.: 111).

3. QuikChange™ Site-Directed™ Mutagenesis

Preparation of non-endogenous human GPCRs can also be accomplished by using QuikChange™ Site-Directed™ Mutagenesis Kit (Stratagene, according to manufacturer's instructions). Endogenous GPCR is preferably used as a template and two mutagenesis primers utilized, as well as, most preferably, a lysine mutagenesis oligonucleotide and a selection marker oligonucleotide (included in kit). For convenience, the codon mutation incorporated into the human GPCR and the respective oligonucleotides are noted, in standard form (Table H):

TABLE H

| | | Lysine Mutagenesis | |
|---|---|---|---|
| | | (SEQ. ID. NO.) | Selection Marker |
| Receptor | Codon | 5′-3′ orientation, mutation | (SEQ. ID. NO.) |
| Identifier | Mutation | underlined | 5′-3′ orientation |
| hCHN3 | S284K | ATGGAGAAAAGAATCAAAAGAA | TATATAGAACATTCTTTT |
| | | TGTTCTATATA (115) | GATTCTTTTCTCCAT |
| | | | (116) |
| hCHN6 | L352K | CGCTCTCTGGCCTTGAAGCGCAC | GCTGAGCGTGCGCTTCA |
| | | GCTCAGC (117) | AGGCCAGAGAGCG (118) |
| hCHN8 | N235K | CCCAGGAAAAAGGTGAAAGTCA | GAAAACTTTGACTTTCAC |
| | | AAGTTTTC (119) | CTTTTTCCTGGG (120) |
| hCHN9 | G223K | GGGGCGCGGGTGAAACGGCTGG | GCTCACCAGCCGTTTCA |
| | | TGAGC (121) | CCCGCGCCCC (122) |
| hCHN10 | L231K | CCCCTTGAAAAGCCTAAGAACTT | GATGACCAAGTTCTTAG |
| | | GGTCATC (123) | GCTTTTCAAGGGG (124) |

### Example 3

Receptor Expression

Although a variety of cells are available to the art for the expression of proteins, it is most preferred that mammalian cells be utilized. The primary reason for this is predicated upon practicalities, i.e., utilization of, e.g., yeast cells for the expression of a GPCR, while possible, introduces into the protocol a non-mammalian cell which may not (indeed, in the case of yeast, does not) include the receptor-coupling, genetic-mechanism and secretary pathways that have evolved for mammalian systems—thus, results obtained in non-mammalian cells, while of potential use, are not as preferred as that obtained from mammalian cells. Of the mammalian cells, COS-7, 293 and 293T cells are particularly preferred, although the specific mammalian cell utilized can be predicated upon the particular needs of the artisan.

On day one, 1×10⁷ 293T cells per 150 mm plate were plated out. On day two, two reaction tubes were prepared (the proportions to follow for each tube are per plate): tube A was prepared by mixing 20 μg DNA (e.g., pCMV vector; pCMV vector with receptor cDNA, etc.) in 1.2 ml serum free DMEM (Irvine Scientific, Irvine, Calif.); tube B was prepared by mixing 120 μl lipofectamine (Gibco BRL) in 1.2 ml serum free DMEM. Tubes A and B were admixed by inversions (several times), followed by incubation at room temperature for 30-45 min. The admixture is referred to as the “transfection mixture”. Plated 293T cells were washed with 1XPBS, followed by addition of 10 ml serum free DMEM. 2.4 ml of the transfection mixture were added to the cells, followed by incubation for 4 hrs at 37° C./5% CO₂. The transfection mixture was removed by aspiration, followed by the addition of 25 ml of DMEM/10% Fetal Bovine Serum. Cells were incubated at 37° C./5% CO₂. After 72 hr incubation, cells were harvested and utilized for analysis.

### Example 4

Assays for Determination of Constitutive Activity of Non-endogenous GPCRs

A variety of approaches are available for assessment of constitutive activity of the non-endogenous human GPCRS. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Constitutively activated receptors continue to exchange GDP for GTP. The non-hydrolyzable GTP analog, [³⁵S]GTPγS, can be utilized to demonstrate enhanced binding of [³⁵S]GTPγS to membranes expressing constitutively activated receptors. The advantage of using [³⁵S]GTPγS binding to measure constitutive activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to known, orphan and constitutively activated G protein-coupled receptors. The assay is generic and has application to drug discovery at all G protein-coupled receptors.

The [³⁵S]GTPγS assay can be incubated in 20 mM HEPES and between 1 and about 20 mM MgCl₂ (this amount can be adjusted for optimization of results, although 20 mM is preferred) pH 7.4, binding buffer with between about 0.3 and about 1.2 nM [³⁵S]GTPγS (this amount can be adjusted for optimization of results, although 1.2 is preferred) and 12.5 to 75 μg membrane protein (e.g, COS-7 cells expressing the receptor; this amount can be adjusted for optimization, although 75 μg is preferred) and 1 μM GDP (this amount can be changed for optimization) for 1 hour. Wheatgerm agglutinin beads (25 μl; Amersham) should then be added and the mixture incubated for another 30 minutes at room temperature. The tubes are then centrifuged at 1500× g for 5 minutes at room temperature and then counted in a scintillation counter.

A less costly but equally applicable alternative has been identified which also meets the needs of large scale screening. Flash plates™ and Wallac™ scintistrips may be utilized to format a high throughput [³⁵S]GTPγS binding assay. Furthermore, using this technique, the assay can be utilized for known GPCRs to simultaneously monitor tritiated ligand binding to the receptor at the same time as monitoring the efficacy via [³⁵S]GTPγS binding. This is possible because the Wallac beta counter can switch energy windows to look at both tritium and ³⁵S-labeled probes. This assay may also be used to detect other types of membrane activation events resulting in receptor activation. For example, the assay may be used to monitor ³²P phosphorylation of a variety of receptors (both G protein coupled and tyrosine kinase receptors). When the membranes are centrifuged to the bottom of the well, the bound [³⁵S]GTPγS or the ³²P-phosphorylated receptor will activate the scintillant which is coated of the wells. Scinti® strips (Wallac) have been used to demonstrate this principle. In addition, the assay also has utility for measuring ligand binding to receptors using radioactively labeled ligands. In a similar manner, when the radiolabeled bound ligand is centrifuged to the bottom of the well, the scintistrip label comes into proximity with the radiolabeled ligand resulting in activation and detection.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells contain a scintillant coating which also contains a specific antibody recognizing cAMP. The cAMP generated in the wells was quantitated by a direct competition for binding of radioactive cAMP tracer to the cAMP antibody. The following serves as a brief protocol for the measurement of changes in cAMP levels in membranes that express the receptors.

Transfected cells are harvested approximately three days after transfection. Membranes were prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000× g for 15 minutes at 4° C. The resulting pellet is then resuspended in buffer containing 20 mM HEPES, pH 7.4 and 0.1 mM EDTA, homogenized for 10 seconds, followed by centrifugation at 49,000× g for 15 minutes at 4° C. The resulting pellet can be stored at −80° C. until utilized. On the day of measurement, the membrane pellet is slowly thawed at room temperature, resuspended in buffer containing 20 mM HEPES, pH 7.4 and 10 mM MgCL₂ (these amounts can be optimized, although the values listed herein are preferred), to yield a final protein concentration of 0.60 mg/ml (the resuspended membranes were placed on ice until use). cAMP standards and Detection Buffer (comprising 2 μCi of tracer [¹²⁵I cAMP (100 μl] to 11 ml Detection Buffer) are prepared and maintained in accordance with the manufacturer's instructions. Assay Buffer is prepared fresh for screening and contained 20 mM HEPES, pH 7.4, 1 mM MgCl₂, 20 mM (Sigma), 0.1 units/ml creatine phosphokinase (Sigma), 50 ,uM GTP (Sigma), and 0.2 mM ATP (Sigma); Assay Buffer can be stored on ice until utilized. The assay is initiated by addition of 50 ul of assay buffer followed by addition of 50 ul of membrane suspension to the NEN Flash Plate. The resultant assay mixture is incubated for 60 minutes at room temperature followed by addition of 100 ul of detection buffer. Plates are then incubated an additional 2-4 hours followed by counting in a Wallac MicroBeta™ scintillation counter. Values of cAMP/well are extrapolated from a standard cAMP curve that is contained within each assay plate.

C. Reporter-based Assays

1. CREB Reporter Assay (Gs-associated Receptors)

A method to detect Gs stimulation depends on the known property of the transcription factor CREB, which is activated in a cAMP-dependent manner. A PathDetect™ CREB trans-Reporting System (Stratagene, Catalogue #219010) can utilized to assay for Gs coupled activity in 293 or 293T cells. Cells are transfected with the plasmids components of this above system and the indicated expression plasmid encoding endogenous or mutant receptor using a Mammalian Transfection Kit (Stratagene, Catalogue #200285) according to the manufacturer's instructions. Briefly, 400 ng pFR-Luc (luciferase reporter plasmid containing Gal4 recognition sequences), 40 ng pFA2-CREB (Gal4-CREB fusion protein containing the Gal4 DNA-binding domain), 80 ng pCMV-receptor expression plasmid (comprising the receptor) and 20 ng CMV-SEAP (secreted alkaline phosphatase expression plasmid; alkaline phosphatase activity is measured in the media of transfected cells to control for variations in transfection efficiency between samples), are combined in a calcium phosphate precipitate as per the Kit's instructions. Half of the precipitate is equally distributed over 3 wells in a 96-well plate, kept on the cells overnight, and replaced with fresh medium the following morning. Forty-eight (48) hr after the start of the transfection, cells are treated and assayed for, e.g., luciferase activity

2. AP1 Reporter Assay (Gq-associated Receptors)

A method to detect Gq stimulation depends on the known property of Gq-dependent phospholipase C to cause the activation of genes containing API elements in their promoter. A Pathdetect™ AP-1 cis-Reporting System (Stratagene, Catalogue #219073) can be utilized following the protocol set forth above with respect to the CREB reporter assay, except that the components of the calcium phosphate precipitate were 410 ng pAP1-Luc, 80 ng pCMV-receptor expression plasmid, and 20 ng CMV-SEAP.

3. CRE-LUC Reporter Assay

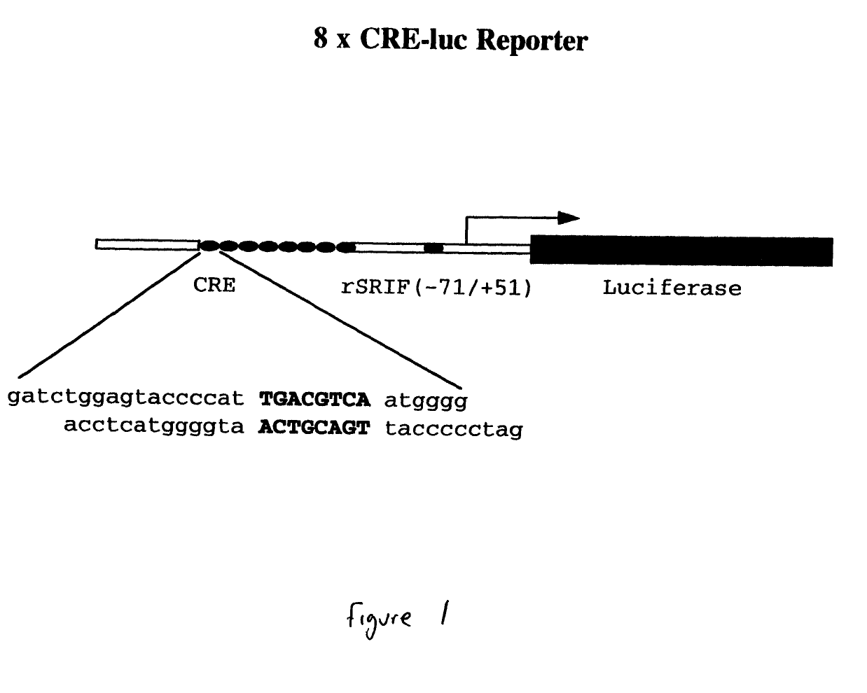

293 and 293T cells are plated-out on 96 well plates at a density of 2×10⁴ cells per well and were transfected using Lipofectamine Reagent (BRL) the following day according to manufacturer instructions. A DNA/lipid mixture is prepared for each 6-well transfection as follows: 260 ng of plasmid DNA in 100 μl of DMEM were gently mixed with 2 μl of lipid in 100 μl of DMEM (the 260 ng of plasmid DNA consisted of 200 ng of a 8×CRE-Luc reporter plasmid (see below and FIG. 1 for a representation of a portion of the plasmid), 50 ng of pCMV comprising endogenous receptor or non-endogenous receptor or pCMV alone, and 10 ng of a GPRS expression plasmid (GPRS in pcDNA3 (Invitrogen)). The 8XCRE-Luc reporter plasmid was prepared as follows: vector SRIF-β-gal was obtained by cloning the rat somatostatin promoter (−71/+51) at BglV-HindIII site in the pβgal-Basic Vector (Clontech). Eight (8) copies of cAMP response element were obtained by PCR from an adenovirus template AdpCF126CCRE8 (see, 7 Human Gene Therapy 1883 (1996)) and cloned into the SRIF-β-gal vector at the Kpn-BglV site, resulting in the 8xCRE-β-gal reporter vector. The 8xCRE-Luc reporter plasmid was generated by replacing the beta-galactosidase gene in the 8xCRE-β-gal reporter vector with the luciferase gene obtained from the pGL3-basic vector (Promega) at the HindIII-BamHI site. Following 30 min. incubation at room temperature, the DNA/lipid mixture was diluted with 400 μl of DMEM and 100 μl of the diluted mixture was added to each well. 100 μl of DMEM with 10% FCS were added to each well after a 4 hr incubation in a cell culture incubator. The following day the transfected cells were changed with 200 μl/well of DMEM with 10% FCS. Eight (8) hours later, the wells were changed to 100 μl /well of DMEM without phenol red, after one wash with PBS. Luciferase activity were measured the next day using the LucLite™ reporter gene assay kit (Packard) following manufacturer instructions and read on a 1450 MicroBeta™ scintillation and luminescence counter (Wallac).

4. SRF-Luc Reporter Assay

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, e.g., COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate, kept on the cells in a serum free media for 24 hours. The last 5 hours the cells are incubated with 1 μM Angiotensin, where indicated. Cells are then lysed and assayed for luciferase activity using a Luclite™ Kit (Packard, Cat. # 601691 1) and “Trilux 1450 Microbeta” liquid scintillation and luminescence counter (Wallac) as per the manufacturer's instructions. The data can be analyzed using GraphPad Prism™ 2.0a (GraphPad Software Inc.).

5. Intracellular IP₃ Accumulation Assay

On day 1, cells comprising the receptors (endogenous and/or non-endogenous) can be plated onto 24 well plates, usually 1×10⁵ cells/well (although his umber can be optimized. On day 2 cells can be transfected by firstly mixing 0.25 ug DNA in 50 ul serum free DMEM/well and 2 ul lipofectamine in 50 μl serumfree DMEM/well. The solutions are gently mixed and incubated for 15-30 min at room temperature. Cells are washed with 0.5 ml PBS and 400 ul of serum free media is mixed with the transfection media and added to the cells. The cells are then incubated for 3-4 hrs at 37° C./5%CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hrs o/n at 37° C./5%CO₂. On Day 4 the cells are washed with 0.5 ml PBS and 0.45 ml of assay medium is added containing inositol-free/serum free media 10 μM pargyline 10 mM lithium chloride or 0.4 ml of assay medium and 50 ul of 10× ketanserin (ket) to final concentration of 10 μM. The cells are then incubated for 30 min at 37° C. The cells are then washed with 0.5 ml PBS and 200 ul of fresh/icecold stop solution (1M KOH; 18 mM Na-borate; 3.8 mM EDTA) is added/well. The solution is kept on ice for 5-10 min or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 sec and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/ 1 M ammonium formate. The columns are regenerated by washing with 10 ml of 0.1 M formic acid/3M ammonium formate and rinsed twice with dd H₂O and stored at 4° C. in water.

Exemplary results are presented below in Table I:

TABLE I

| | | | | Signal | |
|---|---|---|---|---|---|
| | | | Signal | Generated: | |
| | | | Generated: | Non- | |
| | | | Endogenous | Endogenous | |
| | | | Version | Version | |
| | | Assay | (Relative | (Relative | Percent |
| Receptor | Mutation | Utilized | Light Units) | Light Units) | Difference |
| hATL | F239K | SRF-LUC | 34 | 137 | 75%↑ |
| | AT2K2551C3 | SRF-LUC | 34 | 127 | 73%↑ |
| hTDAG8 | 1225K | CRE-LUC | 2,715 | 14,440 | 81%↑ |
| | | (293 cells) | | | |
| | 1225K | CRE-LUC | 65,681 | 185,636 | 65%↑ |
| | | (293T cells) | | | |
| hH9 | F236K | CRE-LUC | 1,887 | 6,096 | 69%↑ |
| hGGKB | V332K | CRE-LUC | 785 | 3,223 | 76%↑ |

C. Cell-based Detection Assay (Example-TDAG8)

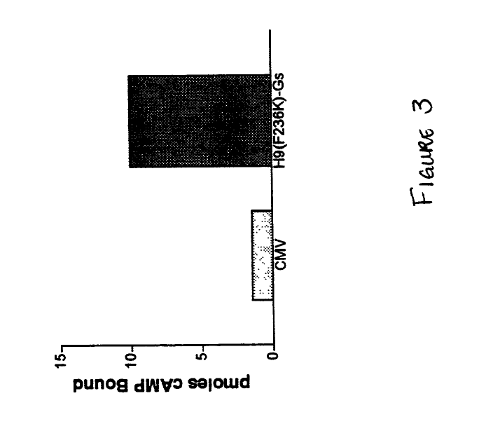

293 cells were plated-out on 150 mm plates at a density of 1.3×10⁷ cells per plate, and were transfected using 12 ug of the respective DNA and 60 ul of Lipofectamine Reagent (BRL) per plate. The transfected cells were grown in media containing serum for an assay performed 24 hours post-transfection. For detection assay performed 48 hours post-transfecfion (assay comparing serum and serum-free media; see FIG. 3), the initial media was changed to either serum or serum-free media. The serum-free media was comprised solely of Dulbecco's Modified Eagle's (DME) High Glucose Medium (Irvine Scientific #9024). In addition to the above DME Medium, the media with serum contained the following: 10% Fetal Bovine Serum (Hyclone #SH30071.03), 1% of 100 mM Sodium Pyruvate (Irvine Scientific #9334), 1% of 20 mM L-Glutamine (Irvine Scientific #9317), and 1% of Penicillin-Streptomycin solution (Irvine Scientific #9366).

A 96-well Adenylyl Cyclase Activation Flashplate™ was used (NEN: #SMP004A). First, 50 ul of the standards for the assay were added to the plate, in duplicate, ranging from concentrations of 50 pmol to zero pmol cAMP per well. The standard cAMP (NEN: #SMP004A) was reconstituted in water, and serial dilutions were made using 1xPBS (Irvine Scientific: #9240). Next, 50 ul of the stimulation buffer (NEN: #SMP004A) was added to all wells. In the case of using compounds to measure activation or inactivation of cAMP, 10 ul of each compound, diluted in water, was added to its respective well, in triplicate. Various final concentrations used range from 1 uM up to 1 mM. Adenosine 5′-triphosphate, ATP, (ResearchBiochemicals International: #A-141) and Adenosine 5′-diphosphate, ADP, (Sigma: #A2754) were used in the assay. Next, the 293 cells transfected with the respective cDNA (CMV or TDAG8) were harvested 24 (assay detection in serum media) or 48 hours post-transfection (assay detection comparing serum and serum-free media). The media was aspirated and the cells washed once with 1xPBS. Then 5 ml of 1xPBS was added to the cells along with 3 ml of cell dissociation buffer (Sigma: #C-1544). The detached cells were transferred to a centrifuge tube and centrifuged at room temperature for five minutes. The supernatant was removed and the cell pellet was resuspended in an appropriate amount of 1xPBS to obtain a final concentration of 2×10⁶ cells permilliliter. To the wells containing the compound, 50 ul of the cells in 1xPBS (1×10⁵ cells/well) were added. The plate was incubated on a shaker for 15 minutes at room temperature. The detection buffer containing the tracer cAMP was prepared. In 11 ml of detection buffer (NEN: #SMP004A), 50 ul (equal to 1 uCi) of [¹²⁵I]cAMP (NEN: #SMP004A) was added. Following incubation, 50 ul of this detection buffer containing tracer cAMP was added to each well. The plate was placed on a shaker and incubated at room temperature for two hours. Finally, the solution from the wells of the plate were aspirated and the flashplate was counted using the Wallac MicroBeta™ scintillation counter.

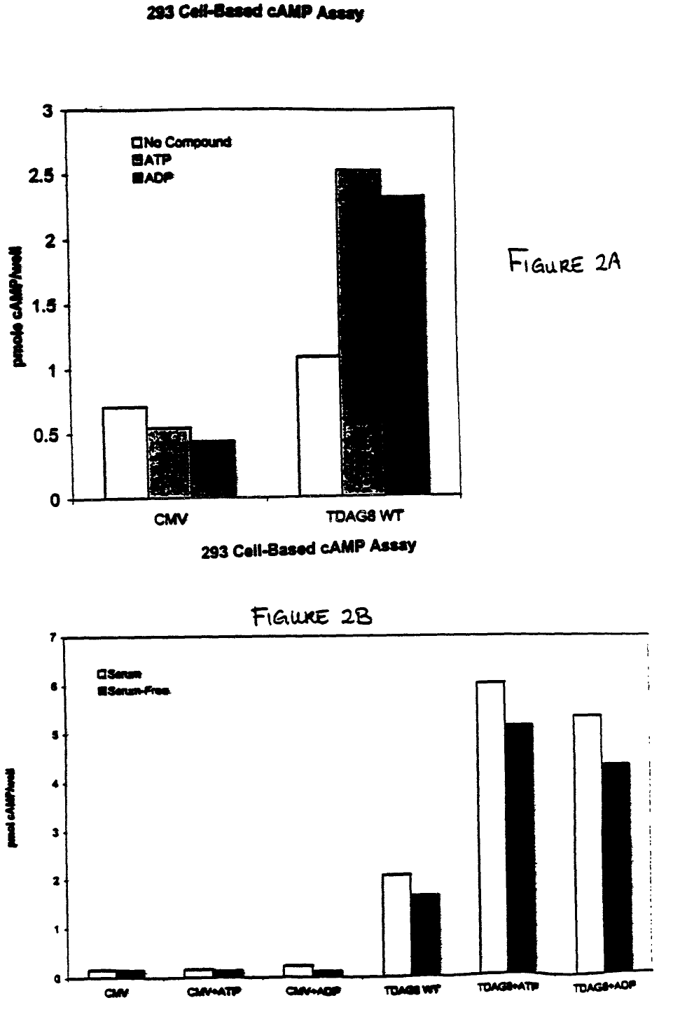

In FIG. 2A, ATP and ADP bind to endogenous TDAG8 resulting in an increase of cAMP of about 59% and about 55% respectively. FIG. 2B evidences ATP and ADP binding to endogenous TDAG8 where endogenous TDAG8 was transfected and grown in serum and serum-free medium. ATP binding to endogenous TDAG8 grown in serum media evidences an increase in cAMP of about 65%, compared to the endogenous TDAG8 with no compounds; in serum-free media there was an increase of about 68%. ADP binding to endogenous TDAG8 in serum evidences about a 61% increase, while in serum-free ADP binding evidences an increase of about 62% increase. ATP and ADP bind to endogenous TDAG8 with an EC50 value of 139.8 uM and 120.5 uM, respectively (data not shown).

Although the results presented in FIG. 2B indicate substantially the same results when serum and serum-free media were compared, our choice is to use a serum based media, although a serum-free media can also be utilized.

### Example 6

GPCR Fusion Protein Preparation

The design of the constitutively activated GPCR-G protein fusion construct was accomplished as follows: both the 5′ and 3′ ends of the rat G protein Gsa (long form; Itoh, H. et al., 83 PNAS 3776 (1986)) were engineered to include a HindIII (5′-AAGCTT-3′) sequence thereon. Following confirmation of the correct sequence (including the flanking HindIII sequences), the entire sequence was shuttled into pcDNA3.1(−) (Invitrogen, cat. no. V795-20) by subcloning using the HindIII restriction site of that vector. The correct orientation for the Gsa sequence was determined after subcloning into pcDNA3.1(−). The modified pcDNA3.1 (−) containing the rat Gsa gene at HindIII sequence was then verified; this vector was now available as a “universal” Gsa protein vector. The pcDNA3.1(−) vector contains a variety of well-known restriction sites upstream of the HindIII site, thus beneficially providing the ability to insert, upstream of the Gs protein, the coding sequence of an endogenous, constitutively active GPCR. This same approach can be utilized to create other “universal” G protein vectors, and, of course, other commercially available or proprietary vectors known to the artisan can be utilized—the important criteria is that the sequence for the GPCR be upstream and in-frame with that of the G protein.

TDAG8 couples via Gs, while H9 couples via Gz. For the following exemplary GPCR Fusion Proteins, fusion to Gsα was accomplished.

A TDAG8(I225K)-Gsa Fusion Protein construct was made as follows: primers were designed as follows:

5′-gatcTCTAGAATGAACAGCACATGTATTGAAG-3′ (SEQ.ID.NO.: 125; sense)

5′-ctagGGTACCCGCTCAAGGACCTCTAATFCCATAG-3′ (SEQ. ID.NO.: 126; antisense).

Nucleotides in lower caps are included as spacers in the restriction sites between the G protein and TDAG8. The sense and anti-sense primers included the restriction sites for XbaI and KpnI, respectively.

PCR was then utilized to secure the respective receptor sequences for fusion within the Gsa universal vector disclosed above, using the following protocol for each: 100 ng cDNA for TDAG8 was added to separate tubes containing 2 ul of each primer (sense and anti-sense), 3 uL of 10 mM dNTPs, 10 uL of 10XTaqPlus™ Precision buffer, 1 uL of TaqPlus™ Precision polymerase (Stratagene: #600211), and 80 uL of water. Reaction temperatures and cycle times for TDAG8 were as follows: the initial denaturing step was done it 94° C. for five minutes, and a cycle of 94° C. for 30 seconds; 55° C. for 30 seconds; 72° C. for two minutes. A final extension time was done at 72° C. for ten minutes. PCR product for was run on a 1% agarose gel and then purified (data not shown). The purified product was digested with XbaI and KpnI (New England Biolabs) and the desired inserts purified and ligated into the Gs universal vector at the respective restriction site. The positive clones was isolated following transformation and determined by restriction enzyme digest; expression using 293 cells was accomplished following the protocol set forth infra. Each positive clone for TDAG8:Gs-Fusion Protein was sequenced to verify correctness.

GPCR Fusion Proteins comprising non-endogenous, constitutively activated TDAG8(I225K) were analyzed as above and verified for constitutive activation.

An H9(F236K)-Gsa Fusion Protein construct was made as follows: primers were designed as follows:

5′-TTAgatatcGGGGCCCACCCTAGCGGT-3′ (SEQ.ID.NO.: 145; sense)

5′-ggtaccCCCACAGCCATITCATCAGGATC-3′ (SEQ.ID.NO.: 146; antisense).

Nucleotides in lower caps are included as spacers in the restriction sites between the G protein and H9. The sense and anti-sense primers included the restriction sites for EcoRV and KpnI, respectively such that spacers (attributed to the restriction sites) exists between the G protein and H9.

PCR was then utilized to secure the respective receptor sequences for fusion within the Gsa universal vector disclosed above, using the following protocol for each: 80 ng cDNA for H9 was added to separate tubes containing 100 ng of each primer (sense and anti-sense), and 45 uL of PCR Supermix™ (Gibco-Brl, LifeTech) (50 ul total reaction volume). Reaction temperatures and cycle times for H9 were as follows: the initial denaturing step was done it 94° C. for one, and a cycle of 94° C. for 30 seconds; 55° C. for 30 seconds; 72° C. for two minutes. A final extension time was done at 72° C. for seven minutes. PCR product for was run on a 1% agarose gel and then purified (data not shown). The purified product was cloned into pCRII-TOPO™ System followed by identification of positive clones. Positive clones were isolated, digested with EcoRV and KpnI (New England Biolabs) and the desired inserts were isolated, purified and ligated into the Gs universal vector at the respective restriction site. The positive clones was isolated following transformation and determined by restriction enzyme digest; expression using 293 cells was accomplished following the protocol set forth infra. Each positive clone for H9(F236K):Gs-Fusion Protein was sequenced to verify correctness. Membranes were frozen (−80° C.) until utilized.

To ascertain the ability of measuring a cAMP response mediated by the Gs protein (even though H9 couples with Gz), the following cAMP membrane assay was utilized, based upon an NEN Adenyl Cyclase Activation Flahplate™ Assay kit (96 well format). “Binding Buffer” consisted of 10 mM HEPES, 100 mM NaCl and 100 mM MgCl (ph 7.4). “Regeneration Buffer” was prepared in Binding Buffer and consisted of 20 mM phosphocreatine, 20 U creatine phosphokinase, 20 uM GTP, 0.2 mM ATP, and 0.6 mM IBMX. “cAMP Standards” were prepared in Binding Buffer as follows:

| cAMP Stock | | Added to | Final Assay Concentration |
|---|---|---|---|
| (5,000 pmol/ml in 2 ml H₂O) | | indicted amount of Binding | (50 ul into 100 ul) |
| in ul | | Buffer | to achieve indicated pmol/well |
| A | 250 | 1 ml | 50 |
| B | 500 of A | 500 ul | 25 |
| C | 500 of B | 500 ul | 12.5 |
| D | 500 of C | 750 ul | 5.0 |
| E | 500 of D | 500 ul | 2.5 |
| F | 500 of E | 500 ul | 1.25 |
| G | 500 of F | 750 ul | 0.5 |

Frozen membranes (both pCMV as control and the non-endogenous H(-Gs Fusion Protein) were thawed (on ice at room temperature until in solution). Membranes were homogenized with a polytron until in suspension (2×15 seconds). Membrane protein concentration was determined using the Bradford Assay Protocol (see infra). Membrane concentration was diluted to 0.5 mg/ml in Regeneration Buffer (final assay concentration −25 ug/well). Thereafter, 50 ul of Binding Buffer was added to each well. For control, 50 ul/well of cAMP standard was added to wells 11 and 12 A-G, with Binding Buffer alone to 12H (on the 96-well format). Thereafter, 50 ul/well of protein was added to the wells and incubated at room temperature (on shaker) for 60 min. 100 ul[¹²⁵I]cAMP in Detection Buffer (see infra) was added to each well (final −50 ul[¹²⁵I]cAMP into 11 ml Detection Buffer). These were incubated for 2 hrs at room temperature. Plates were aspirated with an 8 channel manifold and sealed with plate covers. Results (pmoles cAMP bound) were read in a Wallac™ 1450 on “prot #15). Results are presented in FIG. 3.

The results presented in FIG. 3 indicate that the Gs coupled fusion was able to “drive” the cyclase reaction such that measurement of the consitutive activation of H9(F236K) was viable. Based upon these results, the direct identification of candidate compounds that are inverse agonists, agonists and partial agonists is possible using a cyclase-based assay.

### Example 6

Protocol: Direct Identification of Inverse Agonists and Agonists Using [³⁵S]GTPγS

Although we have utilized endogenous, constitutively active GPCRS for the direct identification of candidate compounds as, e.g., inverse agonists, for reasons that are not altogether understood, intra-assay variation can become exacerbated. Preferably, then, a GPCR Fusion Protein, as disclosed above, is also utilized with a non-endogenous, constitutively activated GPCR. We have determined that when such a protein is used, intra-assay variation appears to be substantially stabilized, whereby an effective signal-to-noise ratio is obtained. This has the beneficial result of allowing for a more robust identification of candidate compounds. Thus, it is preferred that for direct identification, a GPCR Fusion Protein be used and that when utilized, the following assay protocols be utilized.

Membrane Preparation

Membranes comprising the non-endogenous, constitutively active orphan GPCR Fusion Protein of interest and for use in the direct identification of candidate compounds as inverse agonists, agonists or partial agonists are preferably prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4

b. Procedure

All materials are kept on ice throughout the procedure. Firstly, the media is aspirated from a confluent monolayer of cells, followed by rinse with 10 ml cold PBS, followed by aspiration. Thereafter, 5 ml of Membrane Scrape Buffer is added to scrape cells; this is followed by transfer of cellular extract into 50 ml centrifuge tubes (centrifuged at 20,000 rpm for 17 minutes at 4° C.). Thereafter, the supernatant is aspirated and the pellet is resuspended in 30 ml Membrane Wash Buffer followed by centrifuge at 20,000 rpm for 17 minutes at 4° C. The supernatant is then aspirated and the pellet resuspended in Binding Buffer. This is then homogenized using a Brinkman polytron™ homogenizer (15-20 second bursts until the all material is in suspension). This is referred to herein as “Membrane Protein”.

Bradford Protein Assay

Following the homogenization, protein concentration of the membranes is determined using the Bradford Protein Assay (protein can be diluted to about 1.5 mg/ml, aliquoted and frozen (−80° C.) for later use; when frozen, protocol for use is as follows: on the day of the assay, frozen Membrane Protein is thawed at room temperature, followed by vortex and then homogenized with a polytron at about 12×1,000 rpm for about 5-10 seconds; it is noted that for multiple preparations, the homogenizer should be thoroughly cleaned between homoginezation of different preparations).

a. Materials

Binding Buffer (as per above); Bradford Dye Reagent; Bradford Protein Standard are utilized, following manufacturer instructions (Biorad, cat. no. 500-0006).

b. Procedure

Duplicate tubes are prepared, one including the membrane, and one as a control “blank” . Each contained 800 ul Binding Buffer. Thereafter, 10 ul of Bradford Protein Standard (1 mg/ml) is added to each tube, and 10 ul of membrane Protein is then added to just one tube (not the blank). Thereafter, 200 ul of Bradford Dye Reagent is added to each tube, followed by vortex of each. After five (5) minutes, the tubes were re-vortexed and the material therein is transferred to cuvettes. The cuvettes are then read using a CECIL 3041 spectrophotometer, at wavelength 595.

Direct Identification Assay

a. Materials

GDP Buffer consists of 37.5 ml Binding Buffer and 2 mg GDP (Sigma, cat. no. G-7127), followed by a series of dilutions in Binding Buffer to obtain 0.2 uM GDP (final concentration of GDP in each well was 0.1 uM GDP); each well comprising a candidate compound, has a final volume of 200 ul consisting of 100 ul GDP Buffer (final concentration, 0.1 uM GDP), 50 ul Membrane Protein in Binding Buffer, and 50 ul [³⁵S]GTPγS (0.6 nM) in Binding Buffer (2.5 ul [³⁵S]GTPγS per 10 ml Binding Buffer).

b. Procedure

Candidate compounds are preferably screened using a 96-well plate format (these can be frozen at −80° C.). Membrane Protein (or membranes with expression vector excluding the GPCR Fusion Protein, as control), are homogenized briefly until in suspension. Protein concentration is then determined using the Bradford Protein Assay set forth above. Membrane Protein (and control) is then diluted to 0.25 mg/ml in Binding Buffer (final assay concentration, 12.5 ug/well). Thereafter, 100 ul GDP Buffer is added to each well of a Wallac Scintistrip™ (Wallac). A 5 ul pin-tool is then used to transfer 5 ul of a candidate compound into such well (i.e., 5 ul in total assay volume of 200 ul is a 1:40 ratio such that the final screening concentration of the candidate compound is 10 uM). Again, to avoid contamination, after each transfer step the pin tool should be rinsed in three reservoirs comprising water (1×), ethanol (1×) and water (2×)—excess liquid should be shaken from the tool after each rinse and dried with paper and kimwipes. Thereafter, 50 ul of Membrane Protein is added to each well (a control well comprising membranes without the GPCR Fusion Protein is also utilized), and pre-incubated for 5-10 minutes at room temperature. Thereafter, 50 ul of[³⁵S]GTPγS (0.6 nM) in Binding Buffer is added to each well, followed by incubation on a shaker for 60 minutes at room temperature (again, in this example, plates were covered with foil). The assay is then stopped by spinning of the plates at 4000 RPM for 15 minutes at 22° C. The plates are then aspirated with an 8 channel manifold and sealed with plate covers. The plates are then read on a Wallacc 1450 using setting “Prot. #37” (as per manufacturer instructions).

### Example 7

### Protocol: Confirmation Assay

Using an independent assay approach to provide confirmation of a directly identified candidate compound as set forth above, it is preferred that a confirmation assay then be utilized. In this case, the preferred confirmation assay is a cyclase-based assay.

A modified Flash Plate™ Adenylyl Cyclase kit (New England Nuclear; Cat. No. SMP004A) is preferably utilized for confirmation of candidate compounds directly identified as inverse agonists and agonists to non-endogenous, constitutively activated orphan GPCRs in accordance with the following protocol.

Transfected cells are harvested approximately three days after transfection. Membranes are prepared by homogenization of suspended cells in buffer containing 20 mM HEPES, pH 7.4 and 10 mM MgCl₂. Homogenization is performed on ice using a Brinkman Polytron™ for approximately 10 seconds. The resulting homogenate is centrifuged at 49,000× g for 15 minutes at 4° C. The resulting pellet is then resuspended in buffer containing 20 mM HEPES, pH 7.4 and 0.1 mM EDTA, homogenized for 10 seconds, followed by centrifugation at 49,000× g for 15 minutes at 4° C. The resulting pellet can be stored at −800° C. until utilized. On the day of direct identification screening, the membrane pellet is slowly thawed at room temperature, resuspended in buffer containing 20 mM HEPES, pH 7.4 and 10 mM MgCL2, to yield a final protein concentration of 0.60 mg/ml (the resuspended membranes are placed on ice until use).

cAMP standards and Detection Buffer (comprising 2 μCi of tracer [¹²⁵I cAMP (100 μl] to 11 ml Detection Buffer) are prepared and maintained in accordance with the manufacturer's instructions. Assay Buffer is prepared fresh for screening and contained 20 mM HEPES, pH 7.4, 10 mM MgCl₂, 20 mM phospocreatine (Sigma), 0.1 units/ml creatine phosphokinase (Sigma), 50 μM GTP (Sigma), and 0.2 mM ATP (Sigma); Assay Buffer can be stored on ice until utilized.

Candidate compounds identified as per above (if frozen, thawed at room temperature) are added, preferably, to 96-well plate wells (3 μl/well; 12 μM final assay concentration), together with 40 μl Membrane Protein (30 μg/well) and 50 μl of Assay Buffer. This admixture is then incubated for 30 minutes at room temperature, with gentle shaking.

Following the incubation, 100 μl of Detection Buffer is added to each well, followed by incubation for 2-24 hours. Plates are then counted in a Wallac MicroBeta™ plate reader using “Prot. #31” (as per manufacturer instructions).

It is intended that each of the patents, applications, and printed publications mentioned in this patent document be hereby incorporated by reference in their entirety.

As those skilled in the art will appreciate, numerous changes and modifications may be made to the preferred embodiments of the invention without departing from the spirit of the invention. It is intended that all such variations fall within the scope of the invention.

Although a variety of expression vectors are available to those in the art, for purposes of utilization for both the endogenous and non-endogenous human GPCRS, it is most preferred that the vector utilized be pCMV. This vector was deposited with the American Type Culture Collection (ATCC) on Oct. 13, 1998 (10801 University Blvd., Manassas, Va. 20110-2209 USA) under the provisions of the Budapest Treaty for the International Recognition of the Deposit of Microorganisms for the Purpose of Patent Procedure. The DNA was tested by the ATCC and determined to be. The ATCC has assigned the following deposit number to pCMV: ATCC #203351.

What is claimed is:

1. A cDNA encoding a non-endogenous, constitutively activated version of a human G protein-coupled receptor comprising hARE-3(F313K).

2. A non-endogenous version of a human G protein-coupled receptor encoded by the cDNA of claim 1.

3. A Plasmid comprising a Vector and the cDNA of claim 1.

4. A Host Cell comprising the Plasmid of claim 3.

5. A cDNA encoding a non-endogenous, constitutively activated version of a human G protein-coupled receptor comprising hARE-4(V233K)

6. A non-endogenous version of a human G protein-coupled receptor encoded by the cDNA of claim 5.

7. A Plasmid comprising a Vector and the cDNA of claim 5.

8. A Host Cell comprising the Plasmid of claim 7.

9. A cDNA encoding a non-endogenous, constitutively activated version of a human G protein-coupled receptor comprising hARE-5(A240K).

10. A non-endogenous version of a human G protein-coupled receptor encoded by the cDNA of claim 9.

11. A Plasmid comprising a Vector and the cDNA of claim 5.

12. A Host Cell comprising the Plasmid of claim 11.

13. A cDNA encoding a non-endogenous, constitutively activated version of a human G protein-coupled receptor comprising hGPCR14(L257K).

14. A non-endogenous version of a human G protein-coupled receptor encoded by the cDNA of claim 13.

15. A Plasmid comprising a Vector and the cDNA of claim 13.

16. A Host Cell comprising the Plasmid of claim 15.

17. A cDNA encoding a non-endogenous, constitutively activated version of a human G protein-coupled receptor comprising hGPCR27(C283K).

18. A non-endogenous version of a human G protein-coupled receptor encoded by the cDNA of claim 17.

19. A Plasmid comprising a Vector and the cDNA of claim 17.

20. A Host Cell comprising the Plasmid of claim 19.

21. A cDNA encoding a non-endogenous, constitutively activated version of a human G protein-coupled receptor comprising hARE-1(E232K).

22. A non-endogenous version of a human G protein-coupled receptor encoded by the cDNA of claim 21.

23. A Plasmid comprising a Vector and the cDNA of claim 21.

24. A Host Cell comprising the Plasmid of claim 23.

25. A cDNA encoding a non-endogenous, constitutively activated version of a human G protein-coupled receptor comprising hARE-2(G285K).

26. A non-endogenous version of a human G protein-coupled receptor encoded by the cDNA of claim 25.

27. A Plasmid comprising a Vector and the cDNA of claim 25.

28. A Host Cell comprising the Plasmid of claim 27.

29. A cDNA encoding a non-endogenous, constitutively activated version of a human G protein-coupled receptor comprising hPPR1(L239K).

30. A non-endogenous version of a human G protein-coupled receptor encoded by the cDNA of claim 29.

31. A Plasmid comprising a Vector and the cDNA of claim 29.

32. A Host Cell comprising the Plasmid of claim 31.

33. A cDNA encoding a non-endogenous, constitutively activated version of a human G protein-coupled receptor comprising hG2A(K232A).

34. A non-endogenous version of a human G protein-coupled receptor encoded by the cDNA of claim 33.

35. A Plasmid comprising a Vector and the cDNA of claim 33.

36. A Host Cell comprising the Plasmid of claim 35.

37. A cDNA encoding a non-endogenous, constitutively activated version of a human G protein-coupled receptor comprising hRUP3(L224K).

38. A non-endogenous version of a human G protein-coupled receptor encoded by the CDNA of claim 37.

39. A Plasmid comprising a Vector and the cDNA of claim 37.

40. A Host Cell comprising the Plasmid of claim 39.

41. A cDNA encoding a non-endogenous, constitutively activated version of a human G protein-coupled receptor comprising hRUP5(A236K).

42. A non-endogenous version of a human G protein-coupled receptor encoded by the cDNA of claim 41.

43. A Plasmid comprising a Vector and the cDNA of claim 41.

44. A Host Cell comprising the Plasmid of claim 42.

45. A cDNA encoding a non-endogenous, constitutively activated version of a human G protein-coupled receptor comprising hRUP6(N267K)

46. A non-endogenous version of a human G protein-coupled receptor encoded by the cDNA of claim 45.

47. A Plasmid comprising a Vector and the CDNA of claim 45.

48. A Host Cell comprising the Plasmid of claim 47.

49. A cDNA encoding a non-endogenous, constitutively activated version of a human G protein-coupled receptor comprising hRUP7(A302K).

50. A non-endogenous version of a human G protein-coupled receptor encoded by the cDNA of claim 49.

51. A Plasmid comprising a Vector and the CDNA of claim 49.

52. A Host Cell comprising the Plasmid of claim 51.

53. A cDNA encoding a non-endogenous, constitutively activated version of a human G protein-coupled receptor comprising hCHN4(V236K).

54. A non-endogenous version of a human G protein-coupled receptor encoded by the cDNA of claim 53.

55. A Plasmid comprising a Vector and the cDNA of claim 53.

56. A Host Cell comprising the Plasmid of claim 55.

57. A cDNA encoding a non-endogenous, constitutively activated version of a human G protein-coupled receptor comprising hMC4(A244K).

58. A non-endogenous version of a human G protein-coupled receptor encoded by the cDNA of claim 57.

59. A Plasmid comprising a Vector and the cDNA of claim 57.

60. A Host Cell comprising the Plasmid of claim 60.

61. A cDNA encoding a non-endogenous, constitutively activated version of a human G protein-coupled receptor comprising hCHN3(S284K).

62. A non-endogenous version of a human G protein-coupled receptor encoded by the cDNA of claim 61.

63. A Plasmid comprising a Vector and the cDNA of claim 61.

64. A Host Cell comprising the Plasmid of claim 63.

65. A cDNA encoding a non-endogenous, constitutively activated version of a human G protein-coupled receptor comprising hCHN6(L352K).

66. A non-endogenous version of a human G protein-coupled receptor encoded by the cDNA of claim 65.

67. A Plasmid comprising a Vector and the cDNA of claim 65.

68. A Host Cell comprising the Plasmid of claim 67.

69. A cDNA encoding a non-endogenous, constitutively activated version of a human G protein-coupled receptor comprising hCHN8(N235K).

70. A non-endogenous version of a human G protein-coupled receptor encoded by the cDNA of claim 69.

71. A Plasmid comprising a Vector and the cDNA of claim 69.

72. A Host Cell comprising the Plasmid of claim 71.

73. A cDNA encoding a non-endogenous, constitutively activated version of a human G protein-coupled receptor comprising hH9(F236K).

74. A non-endogenous version of a human G protein-coupled receptor encoded by the cDNA of claim 73.

75. A Plasmid comprising a Vector and the cDNA of claim 73.

76. A Host Cell comprising the Plasmid of claim 74.

77. A cDNA encoding a non-endogenous, constitutively activated version of a human G protein-coupled AT1 receptor selected from the group consisting of: hAT1(F239K); hAT1(N111A); hAT1(AT2K255IC3); and hATI(A243+).

78. A non-endogenous version of a human G protein-coupled receptor encoded by a cDNA of claim 77.

79. A Plasmid comprising a Vector and the cDNA of claim 77.

80. A Host Cell comprising the Plasmid of claim 79.


SEQUENCE LISTING (146 sequences)

SEQ ID NO: 1 - DNA, 1260 bp, Homo sapiens
atggtcttctcggcagtgttgactgcgttccataccgggacatccaacacaacatttgtcgtgtatgaaaacacctacatgaatattacactccctccaccattccagcatcctgacctcagtccattgcttagatatagttttgaaaccatggctcccactggtttgagttccttgaccgtgaatagtacagctgtgcccacaacaccagcagcatttaagagcctaaacttgcctcttcagatcaccctttctgctataatgatattcattctgtttgtgtcttttcttgggaacttggttgtttgcctcatggtttaccaaaaagctgccatgaggtctgcaattaacatcctccttgccagcctagcttttgcagacatgttgcttgcagtgctgaacatgccctttgccctggtaactattcttactacccgatggatttttgggaaattcttctgtagggtatctgctatgtttttctggttatttgtgatagaaggagtagccatcctgctcatcattagcatagataggttccttattatagtccagaggcaggataagctaaacccatatagagctaaggttctgattgcagtttcttgggcaacttccttttgtgtagcttttcctttagccgtaggaaaccccgacctgcagataccttcccgagctccccagtgtgtgtttgggtacacaaccaatccaggctaccaggcttatgtgattttgatttctctcatttctttcttcatacccttcctggtaatactgtactcatttatgggcatactcaacacccttcggcacaatgccttgaggatccatagctaccctgaaggtatatgcctcagccaggccagcaaactgggtctcatgagtctgcagagacctttccagatgagcattgacatgggctttaaaacacgtgccttcaccactattttgattctctttgctgtcttcattgtctgctgggccccattcaccacttacagccttgtggcaacattcagtaagcacttttactatcagcacaacttttttgagattagcacctggctactgtggctctgctacctcaagtctgcattgaatccgctgatctactactggaggattaagaaattccatgatgcttgcctggacatgatgcctaagtccttcaagtttttgccgcagctccctggtcacacaaagcgacggatacgtcctagtgctgtctatgtgtgtggggaacatcggacggtggtgtga

SEQ ID NO: 2 - protein, 419 aa, Homo sapiens
MVFSAVLTAFHTGTSNTTFVVYENTYMNITLPPPFQHPDLSPLLRYSFETMAPTGLSSLTVNSTAVPTTPAAFKSLNLPLQITLSAIMIFILFVSFLGNLVVCLMVYQKAAMRSAINILLASLAFADMLLAVLNMPFALVTILTTRWIFGKFFCRVSAMFFWLFVIEGVAILLIISIDRFLIIVQRQDKLNPYRAKVLIAVSWATSFCVAFPLAVGNPDLQIPSRAPQCVFGYTTNPGYQAYVILISLISFFIPFLVILYSFMGILNTLRHNALRIHSYPEGICLSQASKLGLMSLQRPFQMSIDMGFKTRAFTTILILFAVFIVCWAPFTTYSLVATFSKHFYYQHNFFEISTWLLWLCYLKSALNPLIYYWRIKKFHDACLDMMPKSFKFLPQLPGHTKRRIRPSAVYVCGEHRTVV

SEQ ID NO: 3 - DNA, 1119 bp, Homo sapiens
atgttagccaacagctcctcaaccaacagttctgttctcccgtgtcctgactaccgacctacccaccgcctgcacttggtggtctacagcttggtgctggctgccgggctccccctcaacgcgctagccctctgggtcttcctgcgcgcgctgcgcgtgcactcggtggtgagcgtgtacatgtgtaacctggcggccagcgacctgctcttcaccctctcgctgcccgttcgtctctcctactacgcactgcaccactggcccttccccgacctcctgtgccagacgacgggcgccatcttccagatgaacatgtacggcagctgcatcttcctgatgctcatcaacgtggaccgctacgccgccatcgtgcacccgctgcgactgcgccacctgcggcggccccgcgtggcgcggctgctctgcctgggcgtgtgggcgctcatcctggtgtttgccgtgcccgccgcccgcgtgcacaggccctcgcgttgccgctaccgggacctcgaggtgcgcctatgcttcgagagcttcagcgacgagctgtggaaaggcaggctgctgcccctcgtgctgctggccgaggcgctgggcttcctgctgcccctggcggcggtggtctactcgtcgggccgagtcttctggacgctggcgcgccccgacgccacgcagagccagcggcggcggaagaccgtgcgcctcctgctggctaacctcgtcatcttcctgctgtgcttcgtgccctacaacagcacgctggcggtctacgggctgctgcggagcaagctggtggcggccagcgtgcctgcccgcgatcgcgtgcgcggggtgctgatggtgatggtgctgctggccggcgccaactgcgtgctggacccgctggtgtactactttagcgccgagggcttccgcaacaccctgcgcggcctgggcactccgcaccgggccaggacctcggccaccaacgggacgcgggcggcgctcgcgcaatccgaaaggtccgccgtcaccaccgacgccaccaggccggatgccgccagtcaggggctgctccgaccctccgactcccactctctgtcttccttcacacagtgtccccaggattccgccctctga

SEQ ID NO: 4 - protein, 372 aa, Homo sapiens [sequence omitted]

SEQ ID NO: 5 - DNA, 1107 bp, Homo sapiens
atggccaactccacagggctgaacgcctcagaagtcgcaggctcgttggggttgatcctggcagctgtcgtggaggtgggggcactgctgggcaacggcgcgctgctggtcgtggtgctgcgcacgccgggactgcgcgacgcgctctacctggcgcacctgtgcgtcgtggacctgctggcggccgcctccatcatgccgctgggcctgctggccgcaccgccgcccgggctgggccgcgtgcgcctgggccccgcgccatgccgcgccgctcgcttcctctccgccgctctgctgccggcctgcacgctcggggtggccgcacttggcctggcacgctaccgcctcatcgtgcacccgctgcggccaggctcgcggccgccgcctgtgctcgtgctcaccgccgtgtgggccgcggcgggactgctgggcgcgctctccctgctcggcccgccgcccgcaccgccccctgctcctgctcgctgctcggtcctggctgggggcctcgggcccttccggccgctctgggccctgctggccttcgcgctgcccgccctcctgctgctcggcgcctacggcggcatcttcgtggtggcgcgtcgcgctgccctgaggcccccacggccggcgcgcgggtcccgactccgctcggactctctggatagccgcctttccatcttgccgccgctccggcctcgcctgcccgggggcaaggcggccctggccccagcgctggccgtgggccaatttgcagcctgctggctgccttatggctgcgcgtgcctggcgcccgcagcgcgggccgcggaagccgaagcggctgtcacctgggtcgcctactcggccttcgcggctcaccccttcctgtacgggctgctgcagcgccccgtgcgcttggcactgggccgcctctctcgccgtgcactgcctggacctgtgcgggcctgcactccgcaagcctggcacccgcgggcactcttgcaatgcctccagagacccccagagggccctgccgtaggcccttctgaggctccagaacagacccccgagttggcaggagggcggagccccgcataccaggggccacctgagagttctctctcctga

SEQ ID NO: 6 - protein, 368 aa, Homo sapiens
MANSTGLNASEVAGSLGLILAAVVEVGALLGNGALLVVVLRTPGLRDALYLAHLCVVDLLAAASIMPLGLLAAPPPGLGRVRLGPAPCRAARFLSAALLPACTLGVAALGLARYRLIVHPLRPGSRPPPVLVLTAVWAAAGLLGALSLLGPPPAPPPAPARCSVLAGGLGPFRPLWALLAFALPALLLLGAYGGIFVVARRAALRPPRPARGSRLRSDSLDSRLSILPPLRPRLPGGKAALAPALAVGQFAACWLPYGCACLAPAARAAEAEAAVTWVAYSAFAAHPFLYGLLQRPVRLALGRLSRRALPGPVRACTPQAWHPRALLQCLQRPPEGPAVGPSEAPEQTPELAGGRSPAYQGPPESSLS

SEQ ID NO: 7 - DNA, 1008 bp, Homo sapiens
atggaatcatctttctcatttggagtgatccttgctgtcctggcctccctcatcattgctactaacacactagtggctgtggctgtgctgctgttgatccacaagaatgatggtgtcagtctctgcttcaccttgaatctggctgtggctgacaccttgattggtgtggccatctctggcctactcacagaccagctctccagcccttctcggcccacacagaagaccctgtgcagcctgcggatggcatttgtcacttcctccgcagctgcctctgtcctcacggtcatgctgatcacctttgacaggtaccttgccatcaagcagcccttccgctacttgaagatcatgagtgggttcgtggccggggcctgcattgccgggctgtggttagtgtcttacctcattggcttcctcccactcggaatccccatgttccagcagactgcctacaaagggcagtgcagcttctttgctgtatttcaccctcacttcgtgctgaccctctcctgcgttggcttcttcccagccatgctcctctttgtcttcttctactgcgacatgctcaagattgcctccatgcacagccagcagattcgaaagatggaacatgcaggagccatggctggaggttatcgatccccacggactcccagcgacttcaaagctctccgtactgtgtctgttctcattgggagctttgctctatcctggacccccttccttatcactggcattgtgcaggtggcctgccaggagtgtcacctctacctagtgctggaacggtacctgtggctgctcggcgtgggcaactccctgctcaacccactcatctatgcctattggcagaaggaggtgcgactgcagctctaccacatggccctaggagtgaagaaggtgctcacctcattcctcctctttctctcggccaggaattgtggcccagagaggcccagggaaagttcctgtcacatcgtcactatctccagctcagagtttgatggctaa

SEQ ID NO: 8 - protein, 335 aa, Homo sapiens
MESSFSFGVILAVLASLIIATNTLVAVAVLLLIHKNDGVSLCFTLNLAVADTLIGVAISGLLTDQLSSPSRPTQKTLCSLRMAFVTSSAAASVLTVMLITFDRYLAIKQPFRYLKIMSGFVAGACIAGLWLVSYLIGFLPLGIPMFQQTAYKGQCSFFAVFHPHFVLTLSCVGFFPAMLLFVFFYCDMLKIASMHSQQIRKMEHAGAMAGGYRSPRTPSDFKALRTVSVLIGSFALSWTPFLITGIVQVACQECHLYLVLERYLWLLGVGNSLLNPLIYAYWQKEVRLQLYHMALGVKKVLTSFLLFLSARNCGPERPRESSCHIVTISSSEFDG

SEQ ID NO: 9 - DNA, 1413 bp, Homo sapiens
atggacactaccatggaagctgacctgggtgccactggccacaggccccgcacagagcttgatgatgaggactcctacccccaaggtggctgggacacggtcttcctggtggccctgctgctccttgggctgccagccaatgggttgatggcgtggctggccggctcccaggcccggcatggagctggcacgcgtctggcgctgctcctgctcagcctggccctctctgacttcttgttcctggcagcagcggccttccagatcctagagatccggcatgggggacactggccgctggggacagctgcctgccgcttctactacttcctatggggcgtgtcctactcctccggcctcttcctgctggccgccctcagcctcgaccgctgcctgctggcgctgtgcccacactggtaccctgggcaccgcccagtccgcctgcccctctgggtctgcgccggtgtctgggtgctggccacactcttcagcgtgccctggctggtcttccccgaggctgccgtctggtggtacgacctggtcatctgcctggacttctgggacagcgaggagctgtcgctgaggatgctggaggtcctggggggcttcctgcctttcctcctgctgctcgtctgccacgtgctcacccaggccacagcctgtcgcacctgccaccgccaacagcagcccgcagcctgccggggcttcgcccgtgtggccaggaccattctgtcagcctatgtggtcctgaggctgccctaccagctggcccagctgctctacctggccttcctgtgggacgtctactctggctacctgctctgggaggccctggtctactccgactacctgatcctactcaacagctgcctcagccccttcctctgcctcatggccagtgccgacctccggaccctgctgcgctccgtgctctcgtccttcgcggcagctctctgcgaggagcggccgggcagcttcacgcccactgagccacagacccagctagattctgagggtccaactctgccagagccgatggcagaggcccagtcacagatggatcctgtggcccagcctcaggtgaaccccacactccagccacgatcggatcccacagctcagccacagctgaaccctacggcccagccacagtcggatcccacagcccagccacagctgaacctcatggcccagccacagtcagattctgtggcccagccacaggcagacactaacgtccagacccctgcacctgctgccagttctgtgcccagtccctgtgatgaagcttccccaaccccatcctcgcatcctaccccaggggcccttgaggacccagccacacctcctgcctctgaaggagaaagccccagcagcaccccgccagaggcggccccgggcgcaggccccacgtga

SEQ ID NO: 10 - protein, 468 aa, Homo sapiens
MDTTMEADLGATGHRPRTELDDEDSYPQGGWDTVFLVALLLLGLPANGLMAWLAGSQARHGAGTRLALLLLSLALSDFLFLAAAAFQILEIRHGGHWPLGTAACRFYYFLWGVSYSSGLFLLAALSLDRCLLALCPHWYPGHRPVRLPLWVCAGVWVLATLFSVPWLVFPEAAVWWYDLVICLDFWDSEELSLRMLEVLGGFLPFLLLLVCHVLTQATRTCHRQQQPAACRGFARVARTILSAYVVLRLPYQLAQLLYLAFLWDVYSGYLLWEALVYSDYLILLNSCLSPFLCLMASADLRTLLRSVLSSFAAALCEERPGSFTPTEPQTQLDSEGPTLPEPMAEAQSQMDPVAQPQVNPTLQPRSDPTAQPQLNPTAQPQSDPTAQPQLNLMAQPQSDSVAQPQADTNVQTPAPAASSVPSPCDEASPTPSSHPTPGALEDPATPPASEGESPSSTPPEAAPGAGPT

SEQ ID NO: 11 - DNA, 1248 bp, Homo sapiens
atgtcagggatggaaaaacttcagaatgcttcctggatctaccagcagaaactagaagatccattccagaaacacctgaacagcaccgaggagtatctggccttcctctgcggacctcggcgcagccacttcttcctccccgtgtctgtggtgtatgtgccaatttttgtggtgggggtcattggcaatgtcctggtgtgcctggtgattctgcagcaccaggctatgaagacgcccaccaactactacctcttcagcctggcggtctctgacctcctggtcctgctccttggaatgcccctggaggtctatgagatgtggcgcaactaccctttcttgttcgggcccgtgggctgctacttcaagacggccctctttgagaccgtgtgcttcgcctccatcctcagcatcaccaccgtcagcgtggagcgctacgtggccatcctacacccgttccgcgccaaactgcagagcacccggcgccgggccctcaggatcctcggcatcgtctggggcttctccgtgctcttctccctgcccaacaccagcatccatggcatcaagttccactacttccccaatgggtccctggtcccaggttcggccacctgtacggtcatcaagcccatgtggatctacaatttcatcatccaggtcacctccttcctattctacctcctccccatgactgtcatcagtgtcctctactacctcatggcactcagactaaagaaagacaaatctcttgaggcagatgaagggaatgcaaatattcaaagaccctgcagaaaatcagtcaacaagatgctgtttgtcttggtcttagtgtttgctatctgttgggccccgttccacattgaccgactcttcttcagctttgtggaggagtggagtgaatccctggctgctgtgttcaacctcgtccatgtggtgtcaggtgtcttcttctacctgagctcagctgtcaaccccattatctataacctactgtctcgccgcttccaggcagcattccagaatgtgatctcttctttccacaaacagtggcactcccagcatgacccacagttgccacctgcccagcggaacatcttcctgacagaatgccactttgtggagctgaccgaagatataggtccccaattcccatgtcagtcatccatgcacaactctcacctcccaacagccctctctagtgaacagatgtcaagaacaaactatcaaagcttccactttaacaaaacctga

SEQ ID NO: 12 - protein, 415 aa, Homo sapiens [sequence omitted]

SEQ ID NO: 13 - DNA, 1173 bp, Homo sapiens
atgccagatactaatagcacaatcaatttatcactaagcactcgtgttactttagcattttttatgtccttagtagcttttgctataatgctaggaaatgctttggtcattttagcttttgtggtggacaaaaaccttagacatcgaagtagttatttttttcttaacttggccatctctgacttctttgtgggtgtgatctccattcctttgtacatccctcacacgctgttcgaatgggattttggaaaggaaatctgtgtattttggctcactactgactatctgttatgtacagcatctgtatataacattgtcctcatcagctatgatcgatacctgtcagtctcaaatgctgtgtcttatagaactcaacatactggggtcttgaagattgttactctgatggtggccgtttgggtgctggccttcttagtgaatgggccaatgattctagtttcagagtcttggaaggatgaaggtagtgaatgtgaacctggatttttttcggaatggtacatccttgccatcacatcattcttggaattcgtgatcccagtcatcttagtcgcttatttcaacatgaatatttattggagcctgtggaagcgtgatcatctcagtaggtgccaaagccatcctggactgactgctgtctcttccaacatctgtggacactcattcagaggtagactatcttcaaggagatctctttctgcatcgacagaagttcctgcatcctttcattcagagagacagaggagaaagagtagtctcatgttttcctcaagaaccaagatgaatagcaatacaattgcttccaaaatgggttccttctcccaatcagattctgtagctcttcaccaaagggaacatgttgaactgcttagagccaggagattagccaagtcactggccattctcttaggggtttttgctgtttgctgggctccatattctctgttcacaattgtcctttcattttattcctcagcaacaggtcctaaatcagtttggtatagaattgcattttggcttcagtggttcaattcctttgtcaatcctcttttgtatccattgtgtcacaagcgctttcaaaaggctttcttgaaaatattttgtataaaaaagcaacctctaccatcacaacacagtcggtcagtatcttcttaa

SEQ ID NO: 14 - protein, 390 aa, Homo sapiens
MPDTNSTINLSLSTRVTLAFFMSLVAFAIMLGNALVILAFVVDKNLRHRSSYFFLNLAISDFFVGVISIPLYIPHTLFEWDFGKEICVFWLTTDYLLCTASVYNIVLISYDRYLSVSNAVSYRTQHTGVLKIVTLMVAVWVLAFLVNGPMILVSESWKDEGSECEPGFFSEWYILAITSFLEFVIPVILVAYFNMNIYWSLWKRDHLSRCQSHPGLTAVSSNICGHSFRGRLSSRRSLSASTEVPASFHSERQRRKSSLMFSSRTKMNSNTIASKMGSFSQSDSVALHQREHVELLRARRLAKSLAILLGVFAVCWAPYSLFTIVLSFYSSATGPKSVWYRIAFWLQWFNSFVNPLLYPLCHKRFQKAFLKIFCIKKQPLPSQHSRSVSS

SEQ ID NO: 15 - DNA, 30 bp, Artificial Sequence
  Novel Sequence
ggaaagcttaacgatccccaggagcaacat

SEQ ID NO: 16 - DNA, 31 bp, Artificial Sequence
  Novel Sequence
ctgggatcctacgagagcatttttcacacag

SEQ ID NO: 17 - DNA, 1128 bp, Homo sapiens
atggcgaacgcgagcgagccgggtggcagcggcggcggcgaggcggccgccctgggcctcaagctggccacgctcagcctgctgctgtgcgtgagcctagcgggcaacgtgctgttcgcgctgctgatcgtgcgggagcgcagcctgcaccgcgccccgtactacctgctgctcgacctgtgcctggccgacgggctgcgcgcgctcgcctgcctcccggccgtcatgctggcggcgcggcgtgcggcggccgcggcgggggcgccgccgggcgcgctgggctgcaagctgctcgccttcctggccgcgctcttctgcttccacgccgccttcctgctgctgggcgtgggcgtcacccgctacctggccatcgcgcaccaccgcttctatgcagagcgcctggccggctggccgtgcgccgccatgctggtgtgcgccgcctgggcgctggcgctggccgcggccttcccgccagtgctggacggcggtggcgacgacgaggacgcgccgtgcgccctggagcagcggcccgacggcgcccccggcgcgctgggcttcctgctgctgctggccgtggtggtgggcgccacgcacctcgtctacctccgcctgctcttcttcatccacgaccgccgcaagatgcggcccgcgcgcctggtgcccgccgtcagccacgactggaccttccacggcccgggcgccaccggccaggcggccgccaactggacggcgggcttcggccgcgggcccacgccgcccgcgcttgtgggcatccggcccgcagggccgggccgcggcgcgcgccgcctcctcgtgctggaagaattcaagacggagaagaggctgtgcaagatgttctacgccgtcacgctgctcttcctgctcctctgggggccctacgtcgtggccagctacctgcgggtcctggtgcggcccggcgccgtcccccaggcctacctgacggcctccgtgtggctgaccttcgcgcaggccggcatcaaccccgtcgtgtgcttcctcttcaacagggagctgagggactgcttcagggcccagttcccctgctgccagagcccccggaccacccaggcgacccatccctgcgacctgaaaggcattggtttatga

SEQ ID NO: 18 - protein, 375 aa, Homo sapiens
MANASEPGGSGGGEAAALGLKLATLSLLLCVSLAGNVLFALLIVRERSLHRAPYYLLLDLCLADGLRALACLPAVMLAARRAAAAAGAPPGALGCKLLAFLAALFCFHAAFLLLGVGVTRYLAIAHHRFYAERLAGWPCAAMLVCAAWALALAAAFPPVLDGGGDDEDAPCALEQRPDGAPGALGFLLLLAVVVGATHLVYLRLLFFIHDRRKMRPARLVPAVSHDWTFHGPGATGQAAANWTAGFGRGPTPPALVGIRPAGPGRGARRLLVLEEFKTEKRLCKMFYAVTLLFLLLWGPYVVASYLRVLVRPGAVPQAYLTASVWLTFAQAGINPVVCFLFNRELRDCFRAQFPCCQSPRTTQATHPCDLKGIGL

SEQ ID NO: 19 - DNA, 1002 bp, Homo sapiens
atgaacaccacagtgatgcaaggcttcaacagatctgagcggtgccccagagacactcggatagtacagctggtattcccagccctctacacagtggttttcttgaccggcatcctgctgaatactttggctctgtgggtgtttgttcacatccccagctcctccaccttcatcatctacctcaaaaacactttggtggccgacttgataatgacactcatgcttcctttcaaaatcctctctgactcacacctggcaccctggcagctcagagcttttgtgtgtcgtttttcttcggtgatattttatgagaccatgtatgtgggcatcgtgctgttagggctcatagcctttgacagattcctcaagatcatcagacctttgagaaatatttttctaaaaaaacctgtttttgcaaaaacggtctcaatcttcatctggttctttttgttcttcatctccctgccaaatacgatcttgagcaacaaggaagcaacaccatcgtctgtgaaaaagtgtgcttccttaaaggggcctctggggctgaaatggcatcaaatggtaaataacatatgccagtttattttctggactgtttttatcctaatgcttgtgttttatgtggttattgcaaaaaaagtatatgattcttatagaaagtccaaaagtaaggacagaaaaaacaacaaaaagctggaaggcaaagtatttgttgtcgtggctgtcttctttgtgtgttttgctccatttcattttgccagagttccatatactcacagtcaaaccaacaataagactgactgtagactgcaaaatcaactgtttattgctaaagaaacaactctctttttggcagcaactaacatttgtatggatcccttaatatacatattcttatgtaaaaaattcacagaaaagctaccatgtatgcaagggagaaagaccacagcatcaagccaagaaaatcatagcagtcagacagacaacataaccttaggctga

SEQ ID NO: 20 - protein, 333 aa, Homo sapiens
MNTTVMQGFNRSERCPRDTRIVQLVFPALYTVVFLTGILLNTLALWVFVHIPSSSTFIIYLKNTLVADLIMTLMLPFKILSDSHLAPWQLRAFVCRFSSVIFYETMYVGIVLLGLIAFDRFLKIIRPLRNIFLKKPVFAKTVSIFIWFFLFFISLPNTILSNKEATPSSVKKCASLKGPLGLKWHQMVNNICQFIFWTVFILMLVFYVVIAKKVYDSYRKSKSKDRKNNKKLEGKVFVVVAVFFVCFAPFHFARVPYTHSQTNNKTDCRLQNQLFIAKETTLFLAATNICMDPLIYIFLCKKFTEKLPCMQGRKTTASSQENHSSQTDNITLG

SEQ ID NO: 21 - DNA, 1122 bp, Homo sapiens
atggccaacactaccggagagcctgaggaggtgagcggcgctctgtccccaccgtccgcatcagcttatgtgaagctggtactgctgggactgattatgtgcgtgagcctggcgggtaacgccatcttgtccctgctggtgctcaaggagcgtgccctgcacaaggctccttactacttcctgctggacctgtgcctggccgatggcatacgctctgccgtctgcttcccctttgtgctggcttctgtgcgccacggctcttcatggaccttcagtgcactcagctgcaagattgtggcctttatggccgtgctcttttgcttccatgcggccttcatgctgttctgcatcagcgtcacccgctacatggccatcgcccaccaccgcttctacgccaagcgcatgacactctggacatgcgcggctgtcatctgcatggcctggaccctgtctgtggccatggccttcccacctgtctttgacgtgggcacctacaagtttattcgggaggaggaccagtgcatctttgagcatcgctacttcaaggccaatgacacgctgggcttcatgcttatgttggctgtgctcatggcagctacccatgctgtctacggcaagctgctcctcttcgagtatcgtcaccgcaagatgaagccagtgcagatggtgccagccatcagccagaactggacattccatggtcccggggccaccggccaggctgctgccaactggatcgccggctttggccgtgggcccatgccaccaaccctgctgggtatccggcagaatgggcatgcagccagccggcggctactgggcatggacgaggtcaagggtgaaaagcagctgggccgcatgttctacgcgatcacactgctctttctgctcctctggtcaccctacatcgtggcctgctactggcgagtgtttgtgaaagcctgtgctgtgccccaccgctacctggccactgctgtttggatgagcttcgcccaggctgccgtcaacccaattgtctgcttcctgctcaacaaggacctcaagaagtgcctgaccactcacgccccctgctggggcacaggaggtgccccggctcccagagaaccctactgtgtcatgtga

SEQ ID NO: 22 - protein, 373 aa, Homo sapiens
MANTTGEPEEVSGALSPPSASAYVKLVLLGLIMCVSLAGNAILSLLVLKERALHKAPYYFLLDLCLADGIRSAVCFPFVLASVRHGSSWTFSALSCKIVAFMAVLFCFHAAFMLFCISVTRYMAIAHHRFYAKRMTLWTCAAVICMAWTLSVAMAFPPVFDVGTYKFIREEDQCIFEHRYFKANDTLGFMLMLAVLMAATHAVYGKLLLFEYRHRKMKPVQMVPAISQNWTFHGPGATGQAAANWIAGFGRGPMPPTLLGIRQNGHAASRRLLGMDEVKGEKQLGRMFYAITLLFLLLWSPYIVACYWRVFVKACAVPHRYLATAVWMSFAQAAVNPIVCFLLNKDLKKCLTTHAPCWGTGGAPAPREPYCVM

SEQ ID NO: 23 - DNA, 1053 bp, Homo sapiens
atggctttggaacagaaccagtcaacagattattattatgaggaaaatgaaatgaatggcacttatgactacagtcaatatgaattgatctgtatcaaagaagatgtcagagaatttgcaaaagttttcctccctgtattcctcacaatagctttcgtcattggacttgcaggcaattccatggtagtggcaatttatgcctattacaagaaacagagaaccaaaacagatgtgtacatcctgaatttggctgtagcagatttactccttctattcactctgcctttttgggctgttaatgcagttcatgggtgggttttagggaaaataatgtgcaaaataacttcagccttgtacacactaaactttgtctctggaatgcagtttctggcttgcatcagcatagacagatatgtggcagtaactaatgtccccagccaatcaggagtgggaaaaccatgctggatcatctgtttctgtgtctggatggctgccatcttgctgagcataccccagctggttttttatacagtaaatgacaatgctaggtgcattcccattttcccccgctacctaggaacatcaatgaaagcattgattcaaatgctagagatctgcattggatttgtagtaccctttcttattatgggggtgtgctactttatcacggcaaggacactcatgaagatgccaaacattaaaatatctcgacccctaaaagttctgctcacagtcgttatagttttcattgtcactcaactgccttataacattgtcaagttctgccgagccatagacatcatctactccctgatcaccagctgcaacatgagcaaacgcatggacatcgccatccaagtcacagaaagcattgcactctttcacagctgcctcaacccaatcctttatgtttttatgggagcatctttcaaaaactacgttatgaaagtggccaagaaatatgggtcctggagaagacagagacaaagtgtggaggagtttccttttgattctgagggtcctacagagccaaccagtacttttagcatttaa

SEQ ID NO: 24 - protein, 350 aa, Homo sapiens [sequence omitted]

SEQ ID NO: 25 - DNA, 1116 bp, Homo sapiens
atgccaggaaacgccaccccagtgaccaccactgccccgtgggcctccctgggcctctccgccaagacctgcaacaacgtgtccttcgaagagagcaggatagtcctggtcgtggtgtacagcgcggtgtgcacgctgggggtgccggccaactgcctgactgcgtggctggcgctgctgcaggtactgcagggcaacgtgctggccgtctacctgctctgcctggcactctgcgaactgctgtacacaggcacgctgccactctgggtcatctatatccgcaaccagcaccgctggaccctaggcctgctggcctcgaaggtgaccgcctacatcttcttctgcaacatctacgtcagcatcctcttcctgtgctgcatctcctgcgaccgcttcgtggccgtggtgtacgcgctggagagtcggggccgccgccgccggaggaccgccatcctcatctccgcctgcatcttcatcctcgtcgggatcgttcactacccggtgttccagacggaagacaaggagacctgctttgacatgctgcagatggacagcaggattgccgggtactactacgccaggttcaccgttggctttgccatccctctctccatcatcgccttcaccaaccaccggattttcaggagcatcaagcagagcatgggcttaagcgctgcccagaaggccaaggtgaagcactcggccatcgcggtggttgtcatcttcctagtctgcttcgccccgtaccacctggttctcctcgtcaaagccgctgccttttcctactacagaggagacaggaacgccatgtgcggcttggaggaaaggctgtacacagcctctgtggtgtttctgtgcctgtccacggtgaacggcgtggctgaccccattatctacgtgctggccacggaccattcccgccaagaagtgtccagaatccataaggggtggaaagagtggtccatgaagacagacgtcaccaggctcacccacagcagggacaccgaggagctgcagtcgcccgtggcccttgcagaccactacaccttctccaggcccgtgcacccaccagggtcaccatgccctgcaaagaggctgattgaggagtcctgctga

SEQ ID NO: 26 - protein, 371 aa, Homo sapiens
MPGNATPVTTTAPWASLGLSAKTCNNVSFEESRIVLVVVYSAVCTLGVPANCLTAWLALLQVLQGNVLAVYLLCLALCELLYTGTLPLWVIYIRNQHRWTLGLLASKVTAYIFFCNIYVSILFLCCISCDRFVAVVYALESRGRRRRRTAILISACIFILVGIVHYPVFQTEDKETCFDMLQMDSRIAGYYYARFTVGFAIPLSIIAFTNHRIFRSIKQSMGLSAAQKAKVKHSAIAVVVIFLVCFAPYHLVLLVKAAAFSYYRGDRNAMCGLEERLYTASVVFLCLSTVNGVADPIIYVLATDHSRQEVSRIHKGWKEWSMKTDVTRLTHSRDTEELQSPVALADHYTFSRPVHPPGSPCPAKRLIEESC

SEQ ID NO: 27 - DNA, 1113 bp, Homo sapiens
atggcgaactatagccatgcagctgacaacattttgcaaaatctctcgcctctaacagcctttctgaaactgacttccttgggtttcataataggagtcagcgtggtgggcaacctcctgatctccattttgctagtgaaagataagaccttgcatagagcaccttactacttcctgttggatctttgctgttcagatatcctcagatctgcaatttgtttcccatttgtgttcaactctgtcaaaaatggctctacctggacttatgggactctgacttgcaaagtgattgcctttctgggggttttgtcctgtttccacactgctttcatgctcttctgcatcagtgtcaccagatacttagctatcgcccatcaccgcttctatacaaagaggctgaccttttggacgtgtctggctgtgatctgtatggtgtggactctgtctgtggccatggcatttcccccggttttagacgtgggcacttactcattcattagggaggaagatcaatgcaccttccaacaccgctccttcagggctaatgattccttaggatttatgctgcttcttgctctcatcctcctagccacacagcttgtctacctcaagctgatatttttcgtccacgatcgaagaaaaatgaagccagtccagtttgtagcagcagtcagccagaactggacttttcatggtcctggagccagtggccaggcagctgccaattggctagcaggatttggaaggggtcccacaccacccaccttgctgggcatcaggcaaaatgcaaacaccacaggcagaagaaggctattggtcttagacgagttcaaaatggagaaaagaatcagcagaatgttctatataatgacttttctgtttctaaccttgtggggcccctacctggtggcctgttattggagagtttttgcaagagggcctgtagtaccagggggatttctaacagctgctgtctggatgagttttgcccaagcaggaatcaatccttttgtctgcattttctcaaacagggagctgaggcgctgtttcagcacaacccttctttactgcagaaaatccaggttaccaagggaaccttactgtgttatatga

SEQ ID NO: 28 - protein, 370 aa, Homo sapiens
MANYSHAADNILQNLSPLTAFLKLTSLGFIIGVSVVGNLLISILLVKDKTLHRAPYYFLLDLCCSDILRSAICFPFVFNSVKNGSTWTYGTLTCKVIAFLGVLSCFHTAFMLFCISVTRYLAIAHHRFYTKRLTFWTCLAVICMVWTLSVAMAFPPVLDVGTYSFIREEDQCTFQHRSFRANDSLGFMLLLALILLATQLVYLKLIFFVHDRRKMKPVQFVAAVSQNWTFHGPGASGQAAANWLAGFGRGPTPPTLLGIRQNANTTGRRRLLVLDEFKMEKRISRMFYIMTFLFLTLWGPYLVACYWRVFARGPVVPGGFLTAAVWMSFAQAGINPFVCIFSNRELRRCFSTTLLYCRKSRLPREPYCVI

SEQ ID NO: 29 - DNA, 1080 bp, Homo sapiens
atgcaggtcccgaacagcaccggcccggacaacgcgacgctgcagatgctgcggaacccggcgatcgcggtggccctgcccgtggtgtactcgctggtggcggcggtcagcatcccgggcaacctcttctctctgtgggtgctgtgccggcgcatggggcccagatccccgtcggtcatcttcatgatcaacctgagcgtcacggacctgatgctggccagcgtgttgcctttccaaatctactaccattgcaaccgccaccactgggtattcggggtgctgctttgcaacgtggtgaccgtggccttttacgcaaacatgtattccagcatcctcaccatgacctgtatcagcgtggagcgcttcctgggggtcctgtacccgctcagctccaagcgctggcgccgccgtcgttacgcggtggccgcgtgtgcagggacctggctgctgctcctgaccgccctgtgcccgctggcgcgcaccgatctcacctacccggtgcacgccctgggcatcatcacctgcttcgacgtcctcaagtggacgatgctccccagcgtggccatgtgggccgtgttcctcttcaccatcttcatcctgctgttcctcatcccgttcgtgatcaccgtggcttgttacacggccaccatcctcaagctgttgcgcacggaggaggcgcacggccgggagcagcggaggcgcgcggtgggcctggccgcggtggtcttgctggcctttgtcacctgcttcgcccccaacaacttcgtgctcctggcgcacatcgtgagccgcctgttctacggcaagagctactaccacgtgtacaagctcacgctgtgtctcagctgcctcaacaactgtctggacccgtttgtttattactttgcgtcccgggaattccagctgcgcctgcgggaatatttgggctgccgccgggtgcccagagacaccctggacacgcgccgcgagagcctcttctccgccaggaccacgtccgtgcgctccgaggccggtgcgcaccctgaagggatggagggagccaccaggcccggcctccagaggcaggagagtgtgttctga

SEQ ID NO: 30 - protein, 359 aa, Homo sapiens
MQVPNSTGPDNATLQMLRNPAIAVALPVVYSLVAAVSIPGNLFSLWVLCRRMGPRSPSVIFMINLSVTDLMLASVLPFQIYYHCNRHHWVFGVLLCNVVTVAFYANMYSSILTMTCISVERFLGVLYPLSSKRWRRRRYAVAACAGTWLLLLTALCPLARTDLTYPVHALGIITCFDVLKWTMLPSVAMWAVFLFTIFILLFLIPFVITVACYTATILKLLRTEEAHGREQRRRAVGLAAVVLLAFVTCFAPNNFVLLAHIVSRLFYGKSYYHVYKLTLCLSCLNNCLDPFVYYFASREFQLRLREYLGCRRVPRDTLDTRRESLFSARTTSVRSEAGAHPEGMEGATRPGLQRQESVF

SEQ ID NO: 31 - DNA, 1503 bp, Homo sapiens
atggagcgtccctgggaggacagcccaggcccggagggggcagctgagggctcgcctgtgccagtcgccgccggggcgcgctccggtgccgcggcgagtggcacaggctggcagccatgggctgagtgcccgggacccaaggggagggggcaactgctggcgaccgccggccctttgcgtcgctggcccgccccctcgcctgccagctccagccccgcccccggagcggcgtccgctcactcggttcaaggcagcgcgactgcgggtggcgcacgaccagggcgcagaccttggggcgcgcggcccatggagtcggggctgctgcggccggcgccggtgagcgaggtcatcgtcctgcattacaactacaccggcaagctccgcggtgcgagctaccagccgggtgccggcctgcgcgccgacgccgtggtgtgcctggcggtgtgcgccttcatcgtgctagagaatctagccgtgttgttggtgctcggacgccacccgcgcttccacgctcccatgttcctgctcctgggcagcctcacgttgtcggatctgctggcaggcgccgcctacgccgccaacatcctactgtcggggccgctcacgctgaaactgtcccccgcgctctggttcgcacgggagggaggcgtcttcgtggcactcactgcgtccgtgctgagcctcctggccatcgcgctggagcgcagcctcaccatggcgcgcagggggcccgcgcccgtctccagtcgggggcgcacgctggcgatggcagccgcggcctggggcgtgtcgctgctcctcgggctcctgccagcgctgggctggaattgcctgggtcgcctggacgcttgctccactgtcttgccgctctacgccaaggcctacgtgctcttctgcgtgctcgccttcgtgggcatcctggccgcgatctgtgcactctacgcgcgcatctactgccaggtacgcgccaacgcgcggcgcctgccggcacggcccgggactgcggggaccacctcgacccgggcgcgtcgcaagccgcgctctctggccttgctgcgcacgctcagcgtggtgctcctggcctttgtggcatgttggggccccctcttcctgctgctgttgctcgacgtggcgtgcccggcgcgcacctgtcctgtactcctgcaggccgatcccttcctgggactggccatggccaactcacttctgaaccccatcatctacacgctcaccaaccgcgacctgcgccacgcgctcctgcgcctggtctgctgcggacgccactcctgcggcagagacccgagtggctcccagcagtcggcgagcgcggctgaggcttccgggggcctgcgccgctgcctgcccccgggccttgatgggagcttcagcggctcggagcgctcatcgccccagcgcgacgggctggacaccagcggctccacaggcagccccggtgcacccacagccgcccggactctggtatcagaaccggctgcagactga

SEQ ID NO: 32 - protein, 500 aa, Homo sapiens
MERPWEDSPGPEGAAEGSPVPVAAGARSGAAASGTGWQPWAECPGPKGRGQLLATAGPLRRWPAPSPASSSPAPGAASAHSVQGSATAGGARPGRRPWGARPMESGLLRPAPVSEVIVLHYNYTGKLRGASYQPGAGLRADAVVCLAVCAFIVLENLAVLLVLGRHPRFHAPMFLLLGSLTLSDLLAGAAYAANILLSGPLTLKLSPALWFAREGGVFVALTASVLSLLAIALERSLTMARRGPAPVSSRGRTLAMAAAAWGVSLLLGLLPALGWNCLGRLDACSTVLPLYAKAYVLFCVLAFVGILAAICALYARIYCQVRANARRLPARPGTAGTTSTRARRKPRSLALLRTLSVVLLAFVACWGPLFLLLLLDVACPARTCPVLLQADPFLGLAMANSLLNPIIYTLTNRDLRHALLRLVCCGRHSCGRDPSGSQQSASAAEASGGLRRCLPPGLDGSFSGSERSSPQRDGLDTSGSTGSPGAPTAARTLVSEPAAD

SEQ ID NO: 33 - DNA, 1029 bp, Homo sapiens
atgcaagccgtcgacaatctcacctctgcgcctgggaacaccagtctgtgcaccagagactacaaaatcacccaggtcctcttcccactgctctacactgtcctgttttttgttggacttatcacaaatggcctggcgatgaggattttctttcaaatccggagtaaatcaaactttattatttttcttaagaacacagtcatttctgatcttctcatgattctgacttttccattcaaaattcttagtgatgccaaactgggaacaggaccactgagaacttttgtgtgtcaagttacctccgtcatattttatttcacaatgtatatcagtatttcattcctgggactgataactatcgatcgctaccagaagaccaccaggccatttaaaacatccaaccccaaaaatctcttgggggctaagattctctctgttgtcatctgggcattcatgttcttactctctttgcctaacatgattctgaccaacaggcagccgagagacaagaatgtgaagaaatgctctttccttaaatcagagttcggtctagtctggcatgaaatagtaaattacatctgtcaagtcattttctggattaatttcttaattgttattgtatgttatacactcattacaaaagaactgtaccggtcatacgtaagaacgaggggtgtaggtaaagtccccaggaaaaaggtgaacgtcaaagttttcattatcattgctgtattctttatttgttttgttcctttccattttgcccgaattccttacaccctgagccaaacccgggatgtctttgactgcactgctgaaaatactctgttctatgtgaaagagagcactctgtggttaacttccttaaatgcatgcctggatccgttcatctattttttcctttgcaagtccttcagaaattccttgataagtatgctgaagtgccccaattctgcaacatctctgtcccaggacaataggaaaaaagaacaggatggtggtgacccaaatgaagagactccaatgtaa

SEQ ID NO: 34 - protein, 342 aa, Homo sapiens
MQAVDNLTSAPGNTSLCTRDYKITQVLFPLLYTVLFFVGLITNGLAMRIFFQIRSKSNFIIFLKNTVISDLLMILTFPFKILSDAKLGTGPLRTFVCQVTSVIFYFTMYISISFLGLITIDRYQKTTRPFKTSNPKNLLGAKILSVVIWAFMFLLSLPNMILTNRQPRDKNVKKCSFLKSEFGLVWHEIVNYICQVIFWINFLIVIVCYTLITKELYRSYVRTRGVGKVPRKKVNVKVFIIIAVFFICFVPFHFARIPYTLSQTRDVFDCTAENTLFYVKESTLWLTSLNACLDPFIYFFLCKSFRNSLISMLKCPNSATSLSQDNRKKEQDGGDPNEETPM

SEQ ID NO: 35 - DNA, 1077 bp, Homo sapiens
atgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttga

SEQ ID NO: 36 - protein, 358 aa, Homo sapiens
MSVCYRPPGNETLLSWKTSRATGTAFLLLAALLGLPGNGFVVWSLAGWRPARGRPLAATLVLHLALADGAVLLLTPLFVAFLTRQAWPLGQAGCKAVYYVCALSMYASVLLTGLLSLQRCLAVTRPFLAPRLRSPALARRLLLAVWLAALLLAVPAAVYRHLWRDRVCQLCHPSPVHAAAHLSLETLTAFVLPFGLMLGCYSVTLARLRGARWGSGRHGARVGRLVSAIVLAFGLLWAPYHAVNLLQAVAALAPPEGALAKLGGAGQAARAGTTALAFFSSSVNPVLYVFTAGDLLPRAGPRFLTRLFEGSGEARGGGRSREGTMELRTTPQLKVVGQGRGNGDPGGGMEKDGPEWDL

SEQ ID NO: 37 - DNA, 1005 bp, Homo sapiens
atgctggggatcatggcatggaatgcaacttgcaaaaactggctggcagcagaggctgccctggaaaagtactacctttccattttttatgggattgagttcgttgtgggagtccttggaaataccattgttgtttacggctacatcttctctctgaagaactggaacagcagtaatatttatctctttaacctctctgtctctgacttagcttttctgtgcaccctccccatgctgataaggagttatgccaatggaaactggatatatggagacgtgctctgcataagcaaccgatatgtgcttcatgccaacctctataccagcattctctttctcacttttatcagcatagatcgatacttgataattaagtatcctttccgagaacaccttctgcaaaagaaagagtttgctattttaatctccttggccatttgggttttagtaaccttagagttactacccatacttccccttataaatcctgttataactgacaatggcaccacctgtaatgattttgcaagttctggagaccccaactacaacctcatttacagcatgtgtctaacactgttggggttccttattcctctttttgtgatgtgtttcttttattacaagattgctctcttcctaaagcagaggaataggcaggttgctactgctctgccccttgaaaagcctctcaacttggtcatcatggcagtggtaatcttctctgtgctttttacaccctatcacgtcatgcggaatgtgaggatcgcttcacgcctggggagttggaagcagtatcagtgcactcaggtcgtcatcaactccttttacattgtgacacggcctttggcctttctgaacagtgtcatcaaccctgtcttctattttcttttgggagatcacttcagggacatgctgatgaatcaactgagacacaacttcaaatcccttacatcctttagcagatgggctcatgaactcctactttcattcagagaaaagtga

SEQ ID NO: 38 - protein, 334 aa, Homo sapiens
MLGIMAWNATCKNWLAAEAALEKYYLSIFYGIEFVVGVLGNTIVVYGYIFSLKNWNSSNIYLFNLSVSDLAFLCTLPMLIRSYANGNWIYGDVLCISNRYVLHANLYTSILFLTFISIDRYLIIKYPFREHLLQKKEFAILISLAIWVLVTLELLPILPLINPVITDNGTTCNDFASSGDPNYNLIYSMCLTLLGFLIPLFVMCFFYYKIALFLKQRNRQVATALPLEKPLNLVIMAVVIFSVLFTPYHVMRNVRIASRLGSWKQYQCTQVVINSFYIVTRPLAFLNSVINPVFYFLLGDHFRDMLMNQLRHNFKSLTSFSRWAHELLLSFREK

SEQ ID NO: 39 - DNA, 1296 bp, Homo sapiens
atgcaggcgcttaacattaccccggagcagttctctcggctgctgcgggaccacaacctgacgcgggagcagttcatcgctctgtaccggctgcgaccgctcgtctacaccccagagctgccgggacgcgccaagctggccctcgtgctcaccggcgtgctcatcttcgccctggcgctctttggcaatgctctggtgttctacgtggtgacccgcagcaaggccatgcgcaccgtcaccaacatctttatctgctccttggcgctcagtgacctgctcatcaccttcttctgcattcccgtcaccatgctccagaacatttccgacaactggctggggggtgctttcatttgcaagatggtgccatttgtccagtctaccgctgttgtgacagaaatgctcactatgacctgcattgctgtggaaaggcaccagggacttgtgcatccttttaaaatgaagtggcaatacaccaaccgaagggctttcacaatgctaggtgtggtctggctggtggcagtcatcgtaggatcacccatgtggcacgtgcaacaacttgagatcaaatatgacttcctatatgaaaaggaacacatctgctgcttagaagagtggaccagccctgtgcaccagaagatctacaccaccttcatccttgtcatcctcttcctcctgcctcttatggtgatgcttattctgtacagtaaaattggttatgaactttggataaagaaaagagttggggatggttcagtgcttcgaactattcatggaaaagaaatgtccaaaatagccaggaagaagaaacgagctgtcattatgatggtgacagtggtggctctctttgctgtgtgctgggcaccattccatgttgtccatatgatgattgaatacagtaattttgaaaaggaatatgatgatgtcacaatcaagatgatttttgctatcgtgcaaattattggattttccaactccatctgtaatcccattgtctatgcatttatgaatgaaaacttcaaaaaaaatgttttgtctgcagtttgttattgcatagtaaataaaaccttctctccagcacaaaggcatggaaattcaggaattacaatgatgcggaagaaagcaaagttttccctcagagagaatccagtggaggaaaccaaaggagaagcattcagtgatggcaacattgaagtcaaattgtgtgaacagacagaggagaagaaaaagctcaaacgacatcttgctctctttaggtctgaactggctgagaattctcctttagacagtgggcattaa

SEQ ID NO: 40 - protein, 431 aa, Homo sapiens
MQALNITPEQFSRLLRDHNLTREQFIALYRLRPLVYTPELPGRAKLALVLTGVLIFALALFGNALVFYVVTRSKAMRTVTNIFICSLALSDLLITFFCIPVTMLQNISDNWLGGAFICKMVPFVQSTAVVTEMLTMTCIAVERHQGLVHPFKMKWQYTNRRAFTMLGVVWLVAVIVGSPMWHVQQLEIKYDFLYEKEHICCLEEWTSPVHQKIYTTFILVILFLLPLMVMLILYSKIGYELWIKKRVGDGSVLRTIHGKEMSKIARKKKRAVIMMVTVVALFAVCWAPFHVVHMMIEYSNFEKEYDDVTIKMIFAIVQIIGFSNSICNPIVYAFMNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 41 - DNA, 24 bp, Artificial Sequence
  Novel Sequence
ctgtgtacagcagttcgcagagtg

SEQ ID NO: 42 - DNA, 24 bp, Artificial Sequence
  Novel Sequence
gagtgccaggcagagcaggtagac

SEQ ID NO: 43 - DNA, 31 bp, Artificial Sequence
  Novel Sequence
cccgaattcctgcttgctcccagcttggccc

SEQ ID NO: 44 - DNA, 32 bp, Artificial Sequence
  Novel Sequence
tgtggatcctgctgtcaaaggtcccattccgg

SEQ ID NO: 45 - DNA, 20 bp, Artificial Sequence
  Novel Sequence
tcacaatgctaggtgtggtc

SEQ ID NO: 46 - DNA, 22 bp, Artificial Sequence
  Novel Sequence
tgcatagacaatgggattacag

SEQ ID NO: 47 - DNA, 511 bp, Homo sapiens
tcacaatgctaggtgtggtctggctggtggcagtcatcgtaggatcacccatgtggcacgtgcaacaacttgagatcaaatatgacttcctatatgaaaaggaacacatctgctgcttagaagagtggaccagccctgtgcaccagaagatctacaccaccttcatccttgtcatcctcttcctcctgcctcttatggtgatgcttattctgtacgtaaaattggttatgaactttggataaagaaaagagttggggatggttcagtgcttcgaactattcatggaaaagaaatgtccaaaatagccaggaagaagaaacgagctgtcattatgatggtgacagtggtggctctctttgctgtgtgctgggcaccattccatgttgtccatatgatgattgaatacagtaattttgaaaaggaatatgatgatgtcacaatcaagatgatttttgctatcgtgcaaattattggattttccaactccatctgtaatcccattgtctatgca

SEQ ID NO: 48 - DNA, 21 bp, Artificial Sequence
  Novel Sequence
ctgcttagaagagtggaccag

SEQ ID NO: 49 - DNA, 22 bp, Artificial Sequence
  Novel Sequence
ctgtgcaccagaagatctacac

SEQ ID NO: 50 - DNA, 21 bp, Artificial Sequence
  Novel Sequence
caaggatgaaggtggtgtaga

SEQ ID NO: 51 - DNA, 23 bp, Artificial Sequence
  Novel Sequence
gtgtagatcttctggtgcacagg

SEQ ID NO: 52 - DNA, 21 bp, Artificial Sequence
  Novel Sequence
gcaatgcaggtcatagtgagc

SEQ ID NO: 53 - DNA, 27 bp, Artificial Sequence
  Novel Sequence
tggagcatggtgacgggaatgcagaag

SEQ ID NO: 54 - DNA, 27 bp, Artificial Sequence
  Novel Sequence
gtgatgagcaggtcactgagcgccaag

SEQ ID NO: 55 - DNA, 23 bp, Artificial Sequence
  Novel Sequence
gcaatgcaggcgcttaacattac

SEQ ID NO: 56 - DNA, 22 bp, Artificial Sequence
  Novel Sequence
ttgggttacaatctgaagggca

SEQ ID NO: 57 - DNA, 23 bp, Artificial Sequence
  Novel Sequence
actccgtgtccagcaggactctg

SEQ ID NO: 58 - DNA, 24 bp, Artificial Sequence
  Novel Sequence
tgcgtgttcctggaccctcacgtg

SEQ ID NO: 59 - DNA, 29 bp, Artificial Sequence
  Novel Sequence
caggccttggattttaatgtcagggatgg

SEQ ID NO: 60 - DNA, 27 bp, Artificial Sequence
  Novel Sequence
ggagagtcagctctgaaagaattcagg

SEQ ID NO: 61 - DNA, 27 bp, Artificial Sequence
  Novel Sequence
tgatgtgatgccagatactaatagcac

SEQ ID NO: 62 - DNA, 27 bp, Artificial Sequence
  Novel Sequence
cctgattcatttaggtgagattgagac

SEQ ID NO: 63 - DNA, 26 bp, Artificial Sequence
  Novel Sequence
cccaagcttccccaggtgtatttgat

SEQ ID NO: 64 - DNA, 26 bp, Artificial Sequence
  Novel Sequence
gttggatccacataatgcattttctc

SEQ ID NO: 65 - DNA, 1080 bp, Homo sapiens
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatatttttaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtga

SEQ ID NO: 66 - protein, 359 aa, Homo sapiens [sequence omitted]

SEQ ID NO: 67 - DNA, 27 bp, Artificial Sequence
  Novel Sequence
accatgggcagcccctggaacggcagc

SEQ ID NO: 68 - DNA, 39 bp, Artificial Sequence
  Novel Sequence
agaaccaccaccagcaggacgcggacggtctgccggtgg

SEQ ID NO: 69 - DNA, 39 bp, Artificial Sequence
  Novel Sequence
gtccgcgtcctgctggtggtggttctggcatttataatt

SEQ ID NO: 70 - DNA, 33 bp, Artificial Sequence
  Novel Sequence
cctggatccttatcccatcgtcttcacgttagc

SEQ ID NO: 71 - DNA, 26 bp, Artificial Sequence
  Novel Sequence
ctggaattctcctgccagcatggtga

SEQ ID NO: 72 - DNA, 30 bp, Artificial Sequence
  Novel Sequence
gcaggatcctatattgcgtgctctgtcccc

SEQ ID NO: 73 - DNA, 999 bp, Homo sapiens
atggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattatcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcagcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggagcgattaccttgaccatcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcagatattaa

SEQ ID NO: 74 - protein, 332 aa, Homo sapiens [sequence omitted]

SEQ ID NO: 75 - DNA, 32 bp, Artificial Sequence
  Novel Sequence
ccgaagcttcgagctgagtaaggcggcgggct

SEQ ID NO: 76 - DNA, 31 bp, Artificial Sequence
  Novel Sequence
gtggaattcatttgccctgcctcaaccccca

SEQ ID NO: 77 - DNA, 1344 bp, Homo sapiens
atggagctgctaaagctgaaccggagcgtgcagggaaccggacccgggccgggggcttccctgtgccgcccgggggcgcctctcctcaacagcagcagtgtgggcaacctcagctgcgagccccctcgcattcgcggagccgggacacgagaattggagctggccattagaatcactctttacgcagtgatcttcctgatgagcgttggaggaaatatgctcatcatcgtggtcctgggactgagccgccgcctgaggactgtcaccaatgccttcctcctctcactggcagtcagcgacctcctgctggctgtggcttgcatgcccttcaccctcctgcccaatctcatgggcacattcatctttggcaccgtcatctgcaaggcggtttcctacctcatgggggtgtctgtgagtgtgtccacgctaagcctcgtggccatcgcactggagcgatatagcgccatctgccgaccactgcaggcacgagtgtggcagacgcgctcccacgcggctcgcgtgattgtagccacgtggctgctgtccggactactcatggtgccctaccccgtgtacactgtcgtgcaaccagtggggcctcgtgtgctgcagtgcgtgcatcgctggcccagtgcgcgggtccgccagacctggtccgtactgctgcttctgctcttgttcttcatcccaggtgtggttatggccgtggcctacgggcttatctctcgcgagctctacttagggcttcgctttgacggcgacagtgacagcgacagccaaagcagggtccgaaaccaaggcgggctgccaggggctgttcaccagaacgggcgttgccggcctgagactggcgcggttggcaaagacagcgatggctgctacgtgcaacttccacgttcccggcctgccctggagctgacggcgctgacggctcctgggccgggatccggctcccggcccacccaggccaagctgctggctaagaagcgcgtggtgcgaatgttgctggtgatcgttgtgcttttttttctgtgttggttgccagtttatagtgccaacacgtggcgcgcctttgatggcccgggtgcacaccgagcactctcgggtgctcctatctccttcattcacttgctgagctacgcctcggcctgtgtcaaccccctggtctactgcttcatgcaccgtcgctttcgccaggcctgcctggaaacttgcgctcgctgctgcccccggcctccacgagctcgccccagggctcttcccgatgaggaccctcccactccctccattgcttcgctgtccaggcttagctacaccaccatcagcacactgggccctggctga

SEQ ID NO: 78 - protein, 447 aa, Homo sapiens
MELLKLNRSVQGTGPGPGASLCRPGAPLLNSSSVGNLSCEPPRIRGAGTRELELAIRITLYAVIFLMSVGGNMLIIVVLGLSRRLRTVTNAFLLSLAVSDLLLAVACMPFTLLPNLMGTFIFGTVICKAVSYLMGVSVSVSTLSLVAIALERYSAICRPLQARVWQTRSHAARVIVATWLLSGLLMVPYPVYTVVQPVGPRVLQCVHRWPSARVRQTWSVLLLLLLFFIPGVVMAVAYGLISRELYLGLRFDGDSDSDSQSRVRNQGGLPGAVHQNGRCRPETGAVGKDSDGCYVQLPRSRPALELTALTAPGPGSGSRPTQAKLLAKKRVVRMLLVIVVLFFLCWLPVYSANTWRAFDGPGAHRALSVAPISFIHLLSYASACVNPLVYCFMHRRFRQACLETCARCCPRPPRARPRALPDEDPPTPSIASLSRLSYTTISTLGPG

SEQ ID NO: 79 - DNA, 30 bp, Artificial Sequence
  Novel Sequence
tgcaagcttaaaaaggaaaaaatgaacagc

SEQ ID NO: 80 - DNA, 30 bp, Artificial Sequence
  Novel Sequence
taaggatcccttcccttcaaaacatccttg

SEQ ID NO: 81 - DNA, 1014 bp, Homo sapiens
atgaacagcacatgtattgaagaacagcatgacctggatcactatttgtttcccattgtttacatctttgtgattatagtcagcattccagccaatattggatctctgtgtgtgtctttcctgcaacccaagaaggaaagtgaactaggaatttacctcttcagtttgtcactatcagatttactctatgcattaactctccctttatggattgattatacttggaataaagacaactggactttctctcctgccttgtgcaaagggagtgcttttctcatgtacatgaagttttacagcagcacagcattcctcacctgcattgccgttgatcggtatttggctgttgtctaccctttgaagttttttttcctaaggacaagaagaattgcactcatggtcagcctgtccatctggatattggaaaccatcttcaatgctgtcatgttgtgggaagatgaaacagttgttgaatattgcgatgccgaaaagtctaattttactttatgctatgacaaataccctttagagaaatggcaaatcaacctcaacttgttcaggacgtgtacaggctatgcaatacctttggtcaccatcctgatctgtaaccggaaagtctaccaagctgtgcggcacaataaagccacggaaaacaaggaaaagaagagaatcataaaactacttgtcagcatcacagttacttttgtcttatgctttactccctttcatgtgatgttgctgattcgctgcattttagagcatgctgtgaacttcgaagaccacagcaattctgggaagcgaacttacacaatgtatagaatcacggttgcattaacaagtttaaattgtgttgctgatccaattctgtactgttttgttaccgaaacaggaagatatgatatgtggaatatattaaaattctgcactgggaggtgtaatacatcacaaagacaaagaaaacgcatactttctgtgtctacaaaagatactatggaattagaggtccttgagtag

SEQ ID NO: 82 - protein, 337 aa, Homo sapiens
MNSTCIEEQHDLDHYLFPIVYIFVIIVSIPANIGSLCVSFLQPKKESELGIYLFSLSLSDLLYALTLPLWIDYTWNKDNWTFSPALCKGSAFLMYMKFYSSTAFLTCIAVDRYLAVVYPLKFFFLRTRRIALMVSLSIWILETIFNAVMLWEDETVVEYCDAEKSNFTLCYDKYPLEKWQINLNLFRTCTGYAIPLVTILICNRKVYQAVRHNKATENKEKKRIIKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 83 - DNA, 40 bp, Artificial Sequence
  Novel Sequence
caggaagaagaaacgagctgtcattatgatggtgacagtg

SEQ ID NO: 84 - DNA, 40 bp, Artificial Sequence
  Novel Sequence
cactgtcaccatcataatgacagctcgtttcttcttcctg

SEQ ID NO: 85 - DNA, 30 bp, Artificial Sequence
  Novel Sequence
ggccaccggcagaccaaacgcgtcctgctg

SEQ ID NO: 86 - DNA, 31 bp, Artificial Sequence
  Novel Sequence
ctccttcggtcctcctatcgttgtcagaagt

SEQ ID NO: 87 - DNA, 37 bp, Artificial Sequence
  Novel Sequence
ggaaaagaagagaatcaaaaaactacttgtcagcatc

SEQ ID NO: 88 - DNA, 31 bp, Artificial Sequence; identical to SEQ ID NO: 86
  Novel Sequence

SEQ ID NO: 89 - DNA, 1080 bp, Homo sapiens
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatattaaaaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtga

SEQ ID NO: 90 - protein, 359 aa, Homo sapiens
MILNSSTEDGIKRIQDDCPKAGRHNYIFVMIPTLYSIIFVVGIFGNSLVVIVIYFYMKLKTVASVFLLNLALADLCFLLTLPLWAVYTAMEYRWPFGNYLCKIASASVSFNLYASVFLLTCLSIDRYLAIVHPMKSRLRRTMLVAKVTCIIIWLLAGLASLPAIIHRNVFFIENTNITVCAFHYESQNSTLPIGLGLTKNILGFLFPFLIILTSYTLIWKALKKAYEIQKNKPRNDDIKKIIMAIVLFFFFSWIPHQIFTFLDVLIQLGIIRDCRIADIVDTAMPITICIAYFNNCLNPLFYGFLGKKFKRYFLQLLKYIPPKAKSHSNLSTKMSTLSYRPSDNVSSSTKKPAPCFEVE

SEQ ID NO: 91 - DNA, 35 bp, Artificial Sequence
  Novel Sequence
ccaagaaatgatgatattaaaaagataattatggc

SEQ ID NO: 92 - DNA, 31 bp, Artificial Sequence; identical to SEQ ID NO: 86
  Novel Sequence

SEQ ID NO: 93 - DNA, 1080 bp, Homo sapiens
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcgccctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatatttttaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtga

SEQ ID NO: 94 - protein, 359 aa, Homo sapiens
MILNSSTEDGIKRIQDDCPKAGRHNYIFVMIPTLYSIIFVVGIFGNSLVVIVIYFYMKLKTVASVFLLNLALADLCFLLTLPLWAVYTAMEYRWPFGNYLCKIASASVSFALYASVFLLTCLSIDRYLAIVHPMKSRLRRTMLVAKVTCIIIWLLAGLASLPAIIHRNVFFIENTNITVCAFHYESQNSTLPIGLGLTKNILGFLFPFLIILTSYTLIWKALKKAYEIQKNKPRNDDIFKIIMAIVLFFFFSWIPHQIFTFLDVLIQLGIIRDCRIADIVDTAMPITICIAYFNNCLNPLFYGFLGKKFKRYFLQLLKYIPPKAKSHSNLSTKMSTLSYRPSDNVSSSTKKPAPCFEVE

SEQ ID NO: 95 - DNA, 26 bp, Artificial Sequence
  Novel Sequence
cccaagcttccccaggtgtatttgat

SEQ ID NO: 96 - DNA, 29 bp, Artificial Sequence
  Novel Sequence
cctgcaggcgaaactgactctggctgaag

SEQ ID NO: 97 - DNA, 42 bp, Artificial Sequence
  Novel Sequence
ctgtacgctagtgtgtttctactcacgtgtctcagcattgat

SEQ ID NO: 98 - DNA, 26 bp, Artificial Sequence
  Novel Sequence
gttggatccacataatgcattttctc

SEQ ID NO: 99 - DNA, 1080 bp, Homo sapiens
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttattttggaattcgaaaacacttactgaagacgaatagctatgggaagaacaggataacccgtgaccaagttaagaagataattatggcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtga

SEQ ID NO: 100 - protein, 359 aa, Homo sapiens
MILNSSTEDGIKRIQDDCPKAGRHNYIFVMIPTLYSIIFVVGIFGNSLVVIVIYFYMKLKTVASVFLLNLALADLCFLLTLPLWAVYTAMEYRWPFGNYLCKIASASVSFNLYASVFLLTCLSIDRYLAIVHPMKSRLRRTMLVAKVTCIIIWLLAGLASLPAIIHRNVFFIENTNITVCAFHYESQNSTLPIGLGLTKNILGFLFPFLIILTSYFGIRKHLLKTNSYGKNRITRDQVKKIIMAIVLFFFFSWIPHQIFTFLDVLIQLGIIRDCRIADIVDTAMPITICIAYFNNCLNPLFYGFLGKKFKRYFLQLLKYIPPKAKSHSNLSTKMSTLSYRPSDNVSSSTKKPAPCFEVE

SEQ ID NO: 101 - DNA, 37 bp, Artificial Sequence
  Novel Sequence
tccgaattccaaaataacttgtaagaatgatcagaaa

SEQ ID NO: 102 - DNA, 33 bp, Artificial Sequence
  Novel Sequence
agatcttaagaagataattatggcaattgtgct

SEQ ID NO: 103 - DNA, 62 bp, Artificial Sequence
  Novel Sequence
aattcgaaaacacttactgaagacgaatagctatgggaagaacaggataacccgtgaccaag

SEQ ID NO: 104 - DNA, 62 bp, Artificial Sequence
  Novel Sequence
ttaacttggtcacgggttatcctgttcttcccatagctattcgtcttcagtaagtgttttcg

SEQ ID NO: 105 - DNA, 1083 bp, Homo sapiens
atgattctcaactcttctactgaagatggtattaaaagaatccaagatgattgtcccaaagctggaaggcataattacatatttgtcatgattcctactttatacagtatcatctttgtggtgggaatatttggaaacagcttggtggtgatagtcatttacttttatatgaagctgaagactgtggccagtgtttttcttttgaatttagcactggctgacttatgctttttactgactttgccactatgggctgtctacacagctatggaataccgctggccctttggcaattacctatgtaagattgcttcagccagcgtcagtttcaacctgtacgctagtgtgtttctactcacgtgtctcagcattgatcgatacctggctattgttcacccaatgaagtcccgccttcgacgcacaatgcttgtagccaaagtcacctgcatcatcatttggctgctggcaggcttggccagtttgccagctataatccatcgaaatgtatttttcattgagaacaccaatattacagtttgtgctttccattatgagtcccaaaattcaacccttccgatagggctgggcctgaccaaaaatatactgggtttcctgtttccttttctgatcattcttacaagttatactcttatttggaaggccctaaagaaggcttatgaaattcagaagaacaaaccaagaaatgatgatatttttaagataattatggcagcaattgtgcttttctttttcttttcctggattccccaccaaatattcacttttctggatgtattgattcaactaggcatcatacgtgactgtagaattgcagatattgtggacacggccatgcctatcaccatttgtatagcttattttaacaattgcctgaatcctcttttttatggctttctggggaaaaaatttaaaagatattttctccagcttctaaaatatattcccccaaaagccaaatcccactcaaacctttcaacaaaaatgagcacgctttcctaccgcccctcagataatgtaagctcatccaccaagaagcctgcaccatgttttgaggttgagtga

SEQ ID NO: 106 - protein, 360 aa, Homo sapiens
MILNSSTEDGIKRIQDDCPKAGRHNYIFVMIPTLYSIIFVVGIFGNSLVVIVIYFYMKLKTVASVFLLNLALADLCFLLTLPLWAVYTAMEYRWPFGNYLCKIASASVSFNLYASVFLLTCLSIDRYLAIVHPMKSRLRRTMLVAKVTCIIIWLLAGLASLPAIIHRNVFFIENTNITVCAFHYESQNSTLPIGLGLTKNILGFLFPFLIILTSYTLIWKALKKAYEIQKNKPRNDDIFKIIMAAIVLFFFFSWIPHQIFTFLDVLIQLGIIRDCRIADIVDTAMPITICIAYFNNCLNPLFYGFLGKKFKRYFLQLLKYIPPKAKSHSNLSTKMSTLSYRPSDNVSSSTKKPAPCFEVE

SEQ ID NO: 107 - DNA, 26 bp, Artificial Sequence
  Novel Sequence
cccaagcttccccaggtgtatttgat

SEQ ID NO: 108 - DNA, 38 bp, Artificial Sequence
  Novel Sequence
aagcacaattgctgcataattatcttaaaaatatcatc

SEQ ID NO: 109 - DNA, 39 bp, Artificial Sequence
  Novel Sequence
aagataattatggcagcaattgtgcttttctttttcttt

SEQ ID NO: 110 - DNA, 26 bp, Artificial Sequence
  Novel Sequence
gttggatccacataatgcattttctc

SEQ ID NO: 111 - DNA, 1344 bp, Homo sapiens
atggagctgctaaagctgaaccggagcgtgcagggaaccggacccgggccgggggcttccctgtgccgcccgggggcgcctctcctcaacagcagcagtgtgggcaacctcagctgcgagccccctcgcattcgcggagccgggacacgagaattggagctggccattagaatcactctttacgcagtgatcttcctgatgagcgttggaggaaatatgctcatcatcgtggtcctgggactgagccgccgcctgaggactgtcaccaatgccttcctcctctcactggcagtcagcgacctcctgctggctgtggcttgcatgcccttcaccctcctgcccaatctcatgggcacattcatctttggcaccgtcatctgcaaggcggtttcctacctcatgggggtgtctgtgagtgtgtccacgctaagcctcgtggccatcgcactggagcgatatagcgccatctgccgaccactgcaggcacgagtgtggcagacgcgctcccacgcggctcgcgtgattgtagccacgtggctgctgtccggactactcatggtgccctaccccgtgtacactgtcgtgcaaccagtggggcctcgtgtgctgcagtgcgtgcatcgctggcccagtgcgcgggtccgccagacctggtccgtactgctgcttctgctcttgttcttcatcccaggtgtggttatggccgtggcctacgggcttatctctcgcgagctctacttagggcttcgctttgacggcgacagtgacagcgacagccaaagcagggtccgaaaccaaggcgggctgccaggggctgttcaccagaacgggcgttgccggcctgagactggcgcggttggcaaagacagcgatggctgctacgtgcaacttccacgttcccggcctgccctggagctgacggcgctgacggctcctgggccgggatccggctcccggcccacccaggccaagctgctggctaagaagcgcgtgaaacgaatgttgctggtgatcgttgtgcttttttttctgtgttggttgccagtttatagtgccaacacgtggcgcgcctttgatggcccgggtgcacaccgagcactctcgggtgctcctatctccttcattcacttgctgagctacgcctcggcctgtgtcaaccccctggtctactgcttcatgcaccgtcgctttcgccaggcctgcctggaaacttgcgctcgctgctgcccccggcctccacgagctcgccccagggctcttcccgatgaggaccctcccactccctccattgcttcgctgtccaggcttagctacaccaccatcagcacactgggccctggctga

SEQ ID NO: 112 - protein, 447 aa, Homo sapiens
MELLKLNRSVQGTGPGPGASLCRPGAPLLNSSSVGNLSCEPPRIRGAGTRELELAIRITLYAVIFLMSVGGNMLIIVVLGLSRRLRTVTNAFLLSLAVSDLLLAVACMPFTLLPNLMGTFIFGTVICKAVSYLMGVSVSVSTLSLVAIALERYSAICRPLQARVWQTRSHAARVIVATWLLSGLLMVPYPVYTVVQPVGPRVLQCVHRWPSARVRQTWSVLLLLLLFFIPGVVMAVAYGLISRELYLGLRFDGDSDSDSQSRVRNQGGLPGAVHQNGRCRPETGAVGKDSDGCYVQLPRSRPALELTALTAPGPGSGSRPTQAKLLAKKRVKRMLLVIVVLFFLCWLPVYSANTWRAFDGPGAHRALSVAPISFIHLLSYASACVNPLVYCFMHRRFRQACLETCARCCPRPPRARPRALPDEDPPTPSIASLSRLSYTTISTLGPG

SEQ ID NO: 113 - DNA, 34 bp, Artificial Sequence
  Novel Sequence
cagcagcatgcgcttcacgcgcttcttagcccag

SEQ ID NO: 114 - DNA, 35 bp, Artificial Sequence
  Novel Sequence
agaagcgcgtgaagcgcatgctgctggtgatcgtt

SEQ ID NO: 115 - DNA, 33 bp, Artificial Sequence
  Novel Sequence
atggagaaaagaatcaaaagaatgttctatata

SEQ ID NO: 116 - DNA, 33 bp, Artificial Sequence
  Novel Sequence
tatatagaacattcttttgattcttttctccat

SEQ ID NO: 117 - DNA, 30 bp, Artificial Sequence
  Novel Sequence
cgctctctggccttgaagcgcacgctcagc

SEQ ID NO: 118 - DNA, 30 bp, Artificial Sequence
  Novel Sequence
gctgagcgtgcgcttcaaggccagagagcg

SEQ ID NO: 119 - DNA, 30 bp, Artificial Sequence
  Novel Sequence
cccaggaaaaaggtgaaagtcaaagttttc

SEQ ID NO: 120 - DNA, 30 bp, Artificial Sequence
  Novel Sequence
gaaaactttgactttcacctttttcctggg

SEQ ID NO: 121 - DNA, 27 bp, Artificial Sequence
  Novel Sequence
ggggcgcgggtgaaacggctggtgagc

SEQ ID NO: 122 - DNA, 27 bp, Artificial Sequence
  Novel Sequence
gctcaccagccgtttcacccgcgcccc

SEQ ID NO: 123 - DNA, 30 bp, Artificial Sequence
  Novel Sequence
ccccttgaaaagcctaagaacttggtcatc

SEQ ID NO: 124 - DNA, 30 bp, Artificial Sequence
  Novel Sequence
gatgaccaagttcttaggcttttcaagggg

SEQ ID NO: 125 - DNA, 32 bp, Artificial Sequence
  Novel Sequence
gatctctagaatgaacagcacatgtattgaag

SEQ ID NO: 126 - DNA, 35 bp, Artificial Sequence
  Novel Sequence
ctagggtacccgctcaaggacctctaattccatag

SEQ ID NO: 127 - DNA, 1296 bp, Homo sapiens
atgcaggcgcttaacattaccccggagcagttctctcggctgctgcgggaccacaacctgacgcgggagcagttcatcgctctgtaccggctgcgaccgctcgtctacaccccagagctgccgggacgcgccaagctggccctcgtgctcaccggcgtgctcatcttcgccctggcgctctttggcaatgctctggtgttctacgtggtgacccgcagcaaggccatgcgcaccgtcaccaacatctttatctgctccttggcgctcagtgacctgctcatcaccttcttctgcattcccgtcaccatgctccagaacatttccgacaactggctggggggtgctttcatttgcaagatggtgccatttgtccagtctaccgctgttgtgacagaaatgctcactatgacctgcattgctgtggaaaggcaccagggacttgtgcatccttttaaaatgaagtggcaatacaccaaccgaagggctttcacaatgctaggtgtggtctggctggtggcagtcatcgtaggatcacccatgtggcacgtgcaacaacttgagatcaaatatgacttcctatatgaaaaggaacacatctgctgcttagaagagtggaccagccctgtgcaccagaagatctacaccaccttcatccttgtcatcctcttcctcctgcctcttatggtgatgcttattctgtacagtaaaattggttatgaactttggataaagaaaagagttggggatggttcagtgcttcgaactattcatggaaaagaaatgtccaaaatagccaggaagaagaaacgagctaagattatgatggtgacagtggtggctctctttgctgtgtgctgggcaccattccatgttgtccatatgatgattgaatacagtaattttgaaaaggaatatgatgatgtcacaatcaagatgatttttgctatcgtgcaaattattggattttccaactccatctgtaatcccattgtctatgcatttatgaatgaaaacttcaaaaaaaatgttttgtctgcagtttgttattgcatagtaaataaaaccttctctccagcacaaaggcatggaaattcaggaattacaatgatgcggaagaaagcaaagttttccctcagagagaatccagtggaggaaaccaaaggagaagcattcagtgatggcaacattgaagtcaaattgtgtgaacagacagaggagaagaaaaagctcaaacgacatcttgctctctttaggtctgaactggctgagaattctcctttagacagtgggcattaa

SEQ ID NO: 128 - protein, 431 aa, Homo sapiens
MQALNITPEQFSRLLRDHNLTREQFIALYRLRPLVYTPELPGRAKLALVLTGVLIFALALFGNALVFYVVTRSKAMRTVTNIFICSLALSDLLITFFCIPVTMLQNISDNWLGGAFICKMVPFVQSTAVVTEMLTMTCIAVERHQGLVHPFKMKWQYTNRRAFTMLGVVWLVAVIVGSPMWHVQQLEIKYDFLYEKEHICCLEEWTSPVHQKIYTTFILVILFLLPLMVMLILYSKIGYELWIKKRVGDGSVLRTIHGKEMSKIARKKKRAKIMMVTVVALFAVCWAPFHVVHMMIEYSNFEKEYDDVTIKMIFAIVQIIGFSNSICNPIVYAFMNENFKKNVLSAVCYCIVNKTFSPAQRHGNSGITMMRKKAKFSLRENPVEETKGEAFSDGNIEVKLCEQTEEKKKLKRHLALFRSELAENSPLDSGH

SEQ ID NO: 129 - DNA, 2040 bp, Homo sapiens
atgggcagcccctggaacggcagcgacggccccgagggggcgcgggagccgccgtggcccgcgctgccgccttgcgacgagcgccgctgctcgccctttcccctgggggcgctggtgccggtgaccgctgtgtgcctgtgcctgttcgtcgtcggggtgagcggcaacgtggtgaccgtgatgctgatcgggcgctaccgggacatgcggaccaccaccaacttgtacctgggcagcatggccgtgtccgacctactcatcctgctcgggctgccgttcgacctgtaccgcctctggcgctcgcggccctgggtgttcgggccgctgctctgccgcctgtccctctacgtgggcgagggctgcacctacgccacgctgctgcacatgaccgcgctcagcgtcgagcgctacctggccatctgccgcccgctccgcgcccgcgtcttggtcacccggcgccgcgtccgcgcgctcatcgctgtgctctgggccgtggcgctgctctctgccggtcccttcttgttcctggtgggcgtcgagcaggaccccggcatctccgtagtcccgggcctcaatggcaccgcgcggatcgcctcctcgcctctcgcctcgtcgccgcctctctggctctcgcgggcgccaccgccgtccccgccgtcggggcccgagaccgcggaggccgcggcgctgttcagccgcgaatgccggccgagccccgcgcagctgggcgcgctgcgtgtcatgctgtgggtcaccaccgcctacttcttcctgccctttctgtgcctcagcatcctctacgggctcatcgggcgggagctgtggagcagccggcggccgctgcgaggcccggccgcctcggggcgggagagaggccaccggcagaccaaacgcgtcctgcgtaagtggagccgccgtggttccaaagacgcctgcctgcagtccgccccgccggggaccgcgcaaacgctgggtccccttcccctgctcgcccagctctgggcgccgcttccagctccctttcctatttcgattccagcctccacccgccggtacttcccatcccccgagaaaaccatgtcctgtcccccaggagctctgggggaccccagggcgctttgagggtgggatccccggatccgattcagtaaccagcagtgcttttccagagcctctgagaccagaaaggagagttggtaattcttaatccaaccacctgttagatgccacaaatgaggagtcctcacagtgctcttgagaagacgagggagatttcattaagctaaaattttttatttaatgttaagtgatgctgaaggctaaagtaaaccttgctcgtatcaaaaagtaaagattgtgcagacctgttgtagaattcttttcaacagagaacagaaaacttgtctccgaagtgggtttgtggaaggaagcctgccaaggcggcttgttcagagaaattgctccttctggtttatgtccagccttgataacacatatgggagcctactatgcagttttaaagcaagtatccatgcagcctgcagcctggtcattttttctggggtgaggatctgcctaggtagaagttttctctaatttattttgctgttacttgttattgcagatggttccttgtcggggtggggggtttatttgcttcccaatgcttttgttaatcccggtgctgtgtcttatgttgcagtggtggtggttctggcatttataatttgctggttgcccttccacgttggcagaatcatttacataaacacggaagattcgcggatgatgtacttctctcagtactttaacatcgtcgctctgcaacttttctatctgagcgcatctatcaacccaatcctctacaacctcatttcaaagaagtacagagcggcggcctttaaactgctgctcgcaaggaagtccaggccgagaggcttccacagaagcagggacactgcgggggaagttgcaggggacactggaggagacacggtgggctacaccgagacaagcgctaacgtgaagacgatgggataa

SEQ ID NO: 130 - protein, 412 aa, Homo sapiens
MGSPWNGSDGPEGAREPPWPALPPCDERRCSPFPLGALVPVTAVCLCLFVVGVSGNVVTVMLIGRYRDMRTTTNLYLGSMAVSDLLILLGLPFDLYRLWRSRPWVFGPLLCRLSLYVGEGCTYATLLHMTALSVERYLAICRPLRARVLVTRRRVRALIAVLWAVALLSAGPFLFLVGVEQDPGISVVPGLNGTARIASSPLASSPPLWLSRAPPPSPPSGPETAEAAALFSRECRPSPAQLGALRVMLWVTTAYFFLPFLCLSILYGLIGRELWSSRRPLRGPAASGRERGHRQTKRVLLVVVLAFIICWLPFHVGRIIYINTEDSRMMYFSQYFNIVALQLFYLSASINPILYNLISKKYRAAAFKLLLARKSRPRGFHRSRDTAGEVAGDTGGDTVGYTETSANVKTMG

SEQ ID NO: 131 - DNA, 1344 bp, Homo sapiens; identical to SEQ ID NO: 111

SEQ ID NO: 132 - protein, 447 aa, Homo sapiens; identical to SEQ ID NO: 112

SEQ ID NO: 133 - DNA, 1014 bp, Homo sapiens
atgaacagcacatgtattgaagaacagcatgacctggatcactatttgtttcccattgtttacatctttgtgattatagtcagcattccagccaatattggatctctgtgtgtgtctttcctgcaagcaaagaaggaaagtgaactaggaatttacctcttcagtttgtcactatcagatttactctatgcattaactctccctttatggattgattatacttggaataaagacaactggactttctctcctgccttgtgcaaagggagtgcttttctcatgtacatgaatttttacagcagcacagcattcctcacctgcattgccgttgatcggtatttggctgttgtctaccctttgaagttttttttcctaaggacaagaagatttgcactcatggtcagcctgtccatctggatattggaaaccatcttcaatgctgtcatgttgtgggaagatgaaacagttgttgaatattgcgatgccgaaaagtctaattttactttatgctatgacaaataccctttagagaaatggcaaatcaacctcaacttgttcaggacgtgtacaggctatgcaatacctttggtcaccatcctgatctgtaaccggaaagtctaccaagctgtgcggcacaataaagccacggaaaacaaggaaaagaagagaatcaaaaaactacttgtcagcatcacagttacttttgtcttatgctttactccctttcatgtgatgttgctgattcgctgcattttagagcatgctgtgaacttcgaagaccacagcaattctgggaagcgaacttacacaatgtatagaatcacggttgcattaacaagtttaaattgtgttgctgatccaattctgtactgttttgttaccgaaacaggaagatatgatatgtggaatatattaaaattctgcactgggaggtgtaatacatcacaaagacaaagaaaacgcatactttctgtgtctacaaaagatactatggaattagaggtccttgagtag

SEQ ID NO: 134 - protein, 337 aa, Homo sapiens
MNSTCIEEQHDLDHYLFPIVYIFVIIVSIPANIGSLCVSFLQAKKESELGIYLFSLSLSDLLYALTLPLWIDYTWNKDNWTFSPALCKGSAFLMYMNFYSSTAFLTCIAVDRYLAVVYPLKFFFLRTRRFALMVSLSIWILETIFNAVMLWEDETVVEYCDAEKSNFTLCYDKYPLEKWQINLNLFRTCTGYAIPLVTILICNRKVYQAVRHNKATENKEKKRIKKLLVSITVTFVLCFTPFHVMLLIRCILEHAVNFEDHSNSGKRTYTMYRITVALTSLNCVADPILYCFVTETGRYDMWNILKFCTGRCNTSQRQRKRILSVSTKDTMELEVLE

SEQ ID NO: 135 - DNA, 999 bp, Homo sapiens
atggtgaactccacccaccgtgggatgcacacttctctgcacctctggaaccgcagcagttacagactgcacagcaatgccagtgagtcccttggaaaaggctactctgatggagggtgctacgagcaactttttgtctctcctgaggtgtttgtgactctgggtgtcatcagcttgttggagaatatcttagtgattgtggcaatagccaagaacaagaatctgcattcacccatgtactttttcatctgcagcttggctgtggctgatatgctggtgagcgtttcaaatggatcagaaaccattatcatcaccctattaaacagtacagatacggatgcacagagtttcacagtgaatattgataatgtcattgactcggtgatctgtagctccttgcttgcatccatttgcagcctgctttcaattgcagtggacaggtactttactatcttctatgctctccagtaccataacattatgacagttaagcgggttgggatcagcataagttgtatctgggcagcttgcacggtttcaggcattttgttcatcatttactcagatagtagtgctgtcatcatctgcctcatcaccatgttcttcaccatgctggctctcatggcttctctctatgtccacatgttcctgatggccaggcttcacattaagaggattgctgtcctccccggcactggtgccatccgccaaggtgccaatatgaagggaaaaattaccttgaccatcctgattggcgtctttgttgtctgctgggccccattcttcctccacttaatattctacatctcttgtcctcagaatccatattgtgtgtgcttcatgtctcactttaacttgtatctcatactgatcatgtgtaattcaatcatcgatcctctgatttatgcactccggagtcaagaactgaggaaaaccttcaaagagatcatctgttgctatcccctgggaggcctttgtgacttgtctagcagatattaa

SEQ ID NO: 136 - protein, 332 aa, Homo sapiens
MVNSTHRGMHTSLHLWNRSSYRLHSNASESLGKGYSDGGCYEQLFVSPEVFVTLGVISLLENILVIVAIAKNKNLHSPMYFFICSLAVADMLVSVSNGSETIIITLLNSTDTDAQSFTVNIDNVIDSVICSSLLASICSLLSIAVDRYFTIFYALQYHNIMTVKRVGISISCIWAACTVSGILFIIYSDSSAVIICLITMFFTMLALMASLYVHMFLMARLHIKRIAVLPGTGAIRQGANMKGKITLTILIGVFVVCWAPFFLHLIFYISCPQNPYCVCFMSHFNLYLILIMCNSIIDPLIYALRSQELRKTFKEIICCYPLGGLCDLSSRY

SEQ ID NO: 137 - DNA, 33 bp, Artificial Sequence
  Novel Sequence
gccaatatgaagggaaaaattaccttgaccatc

SEQ ID NO: 138 - DNA, 31 bp, Artificial Sequence; identical to SEQ ID NO: 86
  Novel Sequence

SEQ ID NO: 139 - DNA, 1842 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaattttctaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttccctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaagggtgactctgtccatttcaagggtgactctgtccatttcaagcctgactctgttcatttcaagcctgcttccagcaaccccaagcccatcactggccaccatgtctctgctggcagccactccaagtctgccttcagtgctgccaccagccaccctaaacccatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtga

SEQ ID NO: 140 - protein, 613 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNFLTMFVIFLLFAVCWCPINVLTVLVAVSPKEMAGKIPNWLYLAAYFIAYFNSCLNAVIYGLLNENFRREYWTIFHAMRHPIIFFPGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKGDSVHFKGDSVHFKPDSVHFKPASSNPKPITGHHVSAGSHSKSAFSAATSHPKPIKPATSHAEPTTADYPKPATTSHPKPAAADNPELSASHCPEIPAIAHPVSDDSDLPESASSPAAGPTKPAASQLESDTIADLPDPTVVTTSTNDYHDVVVVDVEDDPDEMAV

SEQ ID NO: 141 - DNA, 1842 bp, Homo sapiens
atggggcccaccctagcggttcccaccccctatggctgtattggctgtaagctaccccagccagaatacccaccggctctaatcatctttatgttctgcgcgatggttatcaccatcgttgtagacctaatcggcaactccatggtcattttggctgtgacgaagaacaagaagctccggaattctggcaacatcttcgtggtcagtctctctgtggccgatatgctggtggccatctacccataccctttgatgctgcatgccatgtccattgggggctgggatctgagccagttacagtgccagatggtcgggttcatcacagggctgagtgtggtcggctccatcttcaacatcgtggcaatcgctatcaaccgttactgctacatctgccacagcctccagtacgaacggatcttcagtgtgcgcaatacctgcatctacctggtcatcacctggatcatgaccgtcctggctgtcctgcccaacatgtacattggcaccatcgagtacgatcctcgcacctacacctgcatcttcaactatctgaacaaccctgtcttcactgttaccatcgtctgcatccacttcgtcctccctctcctcatcgtgggtttctgctacgtgaggatctggaccaaagtgctggcggcccgtgaccctgcagggcagaatcctgacaaccaacttgctgaggttcgcaataaactaaccatgtttgtgatcttcctcctctttgcagtgtgctggtgccctatcaacgtgctcactgtcttggtggctgtcagtccgaaggagatggcaggcaagatccccaactggctttatcttgcagcctacttcatagcctacttcaacagctgcctcaacgctgtgatctacgggctcctcaatgagaatttccgaagagaatactggaccatcttccatgctatgcggcaccctatcatattcttctctggcctcatcagtgatattcgtgagatgcaggaggcccgtaccctggcccgcgcccgtgcccatgctcgcgaccaagctcgtgaacaagaccgtgcccatgcctgtcctgctgtggaggaaaccccgatgaatgtccggaatgttccattacctggtgatgctgcagctggccaccccgaccgtgcctctggccaccctaagccccattccagatcctcctctgcctatcgcaaatctgcctctacccaccacaagtctgtctttagccactccaaggctgcctctggtcacctcaagcctgtctctggccactccaagcctgcctctggtcaccccaagtctgccactgtctaccctaagcctgcctctgtccatttcaaggctgactctgtccatttcaagggtgactctgtccatttcaagcctgactctgttcatttcaagcctgcttccagcaaccccaagcccatcactggccaccatgtctctgctggcagccactccaagtctgccttcaatgctgccaccagccaccctaaacccatcaagccagctaccagccatgctgagcccaccactgctgactatcccaagcctgccactaccagccaccctaagcccgctgctgctgacaaccctgagctctctgcctcccattgccccgagatccctgccattgcccaccctgtgtctgacgacagtgacctccctgagtcggcctctagccctgccgctgggcccaccaagcctgctgccagccagctggagtctgacaccatcgctgaccttcctgaccctactgtagtcactaccagtaccaatgattaccatgatgtcgtggttgttgatgttgaagatgatcctgatgaaatggctgtgtga

SEQ ID NO: 142 - protein, 613 aa, Homo sapiens
MGPTLAVPTPYGCIGCKLPQPEYPPALIIFMFCAMVITIVVDLIGNSMVILAVTKNKKLRNSGNIFVVSLSVADMLVAIYPYPLMLHAMSIGGWDLSQLQCQMVGFITGLSVVGSIFNIVAIAINRYCYICHSLQYERIFSVRNTCIYLVITWIMTVLAVLPNMYIGTIEYDPRTYTCIFNYLNNPVFTVTIVCIHFVLPLLIVGFCYVRIWTKVLAARDPAGQNPDNQLAEVRNKLTMFVIFLLFAVCWCPINVLTVLVAVSPKEMAGKIPNWLYLAAYFIAYFNSCLNAVIYGLLNENFRREYWTIFHAMRHPIIFFSGLISDIREMQEARTLARARAHARDQAREQDRAHACPAVEETPMNVRNVPLPGDAAAGHPDRASGHPKPHSRSSSAYRKSASTHHKSVFSHSKAASGHLKPVSGHSKPASGHPKSATVYPKPASVHFKADSVHFKGDSVHFKPDSVHFKPASSNPKPITGHHVSAGSHSKSAFNAATSHPKPIKPATSHAEPTTADYPKPATTSHPKPAAADNPELSASHCPEIPAIAHPVSDDSDLPESASSPAAGPTKPAASQLESDTIADLPDPTVVTTSTNDYHDVVVVDVEDDPDEMAV

SEQ ID NO: 143 - DNA, 33 bp, Artificial Sequence
  Novel Sequence
gctgaggttcgcaataaactaaccatgtttgtg

SEQ ID NO: 144 - DNA, 31 bp, Artificial Sequence; identical to SEQ ID NO: 86
  Novel Sequence

SEQ ID NO: 145 - DNA, 27 bp, Artificial Sequence
  Novel Sequence
ttagatatcggggcccaccctagcggt

SEQ ID NO: 146 - DNA, 29 bp, Artificial Sequence
  Novel Sequence
ggtacccccacagccatttcatcaggatc